SEQUENCE LISTING (95 sequences)

SEQ ID NO: 1 - DNA, 558 bp, synthetic construct
  misc_feature 1..558: note = Mycobacterium smegmatis porin A mutant (D90N/D91N/D93N/D118R/D134R/E139K)
  source 1..558: mol_type = other DNA
atgggtctggataatgaactgagcctggtggacggtcaagatcgtaccctgacggtgcaacaatgggatacctttctgaatggcgtttttccgctggatcgtaatcgcctgacccgtgaatggtttcattccggtcgcgcaaaatatatcgtcgcaggcccgggtgctgacgaattcgaaggcacgctggaactgggttatcagattggctttccgtggtcactgggcgttggtatcaacttctcgtacaccacgccgaatattctgatcaacaatggtaacattaccgcaccgccgtttggcctgaacagcgtgattacgccgaacctgtttccgggtgttagcatctctgcccgtctgggcaatggtccgggcattcaagaagtggcaacctttagtgtgcgcgtttccggcgctaaaggcggtgtcgcggtgtctaacgcccacggtaccgttacgggcgcggccggcggtgtcctgctgcgtccgttcgcgcgcctgattgcctctaccggcgacagcgttacgacctatggcgaaccgtggaatatgaactaa

SEQ ID NO: 2 - protein, 184 aa, synthetic construct
  REGION 1..184: note = Mycobacterium smegmatis porin A mutant (D90N/D91N/D93N/D118R/D134R/E139K)
GLDNELSLVDGQDRTLTVQQWDTFLNGVFPLDRNRLTREWFHSGRAKYIVAGPGADEFEGTLELGYQIGFPWSLGVGINFSYTTPNILINNGNITAPPFGLNSVITPNLFPGVSISARLGNGPGIQEVATFSVRVSGAKGGVAVSNAHGTVTGAAGGVLLRPFARLIASTGDSVTTYGEPWNMN

SEQ ID NO: 3 - DNA, 885 bp, synthetic construct
  misc_feature 1..885: note = alpha-hemolysin mutant E111N/K147N
  source 1..885: mol_type = other DNA
atggcagattctgatattaatattaaaaccggtactacagatattggaagcaatactacagtaaaaacaggtgatttagtcacttatgataaagaaaatggcatgcacaaaaaagtattttatagttttatcgatgataaaaatcacaataaaaaactgctagttattagaacaaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaaaactatatgagtactttaacttatggattcaacggtaatgttactggtgatgatacaggaaaaattggcggccttattggtgcaaatgtttcgattggtcatacactgaactatgttcaacctgatttcaaaacaattttagagagcccaactgataaaaaagtaggctggaaagtgatatttaacaatatggtgaatcaaaattggggaccatacgatcgagattcttggaacccggtatatggcaatcaacttttcatgaaaactagaaatggttctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgattaccaattgcattggacttcaacaaattggaaaggtaccaatactaaagataaatggacagatcgttcttcagaaagatataaaatcgattgggaaaaagaagaaatgacaaattaa

SEQ ID NO: 4 - protein, 293 aa, synthetic construct
  REGION 1..293: note = alpha-hemolysin mutant E111N/K147N
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKNYMSTLTYGFNGNVTGDDTGKIGGLIGANVSIGHTLNYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 5 - protein, 184 aa, Mycobacterium smegmatis
GLDNELSLVDGQDRTLTVQQWDTFLNGVFPLDRNRLTREWFHSGRAKYIVAGPGADEFEGTLELGYQIGFPWSLGVGINFSYTTPNILIDDGDITAPPFGLNSVITPNLFPGVSISADLGNGPGIQEVATFSVDVSGPAGGVAVSNAHGTVTGAAGGVLLRPFARLIASTGDSVTTYGEPWNMN

SEQ ID NO: 6 - protein, 184 aa, Mycobacterium smegmatis
GLDNELSLVDGQDRTLTVQQWDTFLNGVFPLDRNRLTREWFHSGRAKYIVAGPGADEFEGTLELGYQIGFPWSLGVGINFSYTTPNILIDDGDITGPPFGLESVITPNLFPGVSISADLGNGPGIQEVATFSVDVSGPAGGVAVSNAHGTVTGAAGGVLLRPFARLIASTGDSVTTYGEPWNMN

SEQ ID NO: 7 - protein, 183 aa, Mycobacterium smegmatis
VDNQLSVVDGQGRTLTVQQAETFLNGVFPLDRNRLTREWFHSGRATYHVAGPGADEFEGTLELGYQVGFPWSLGVGINFSYTTPNILIDGGDITQPPFGLDTIITPNLFPGVSISADLGNGPGIQEVATFSVDVKGAKGAVAVSNAHGTVTGAAGGVLLRPFARLIASTGDSVTTYGEPWNMN

SEQ ID NO: 8 - protein, 8 aa, synthetic construct
  REGION 1..8: note = Amino acid sequence of the Hel308 motif
  VARIANT 2: note = X = C, M or L
  VARIANT 3: note = X = any amino acid
QXXGRAGR

SEQ ID NO: 9 - protein, 9 aa, synthetic construct
  REGION 1..9: note = Amino acid sequence of the extended Hel308 motif
  VARIANT 2: note = X = C, M or L
  VARIANT 3: note = X = any amino acid
QXXGRAGRP

SEQ ID NO: 10 - protein, 760 aa, Methanococcoides burtonii
MMIRELDIPRDIIGFYEDSGIKELYPPQAEAIEMGLLEKKNLLAAIPTASGKTLLAELAMIKAIREGGKALYIVPLRALASEKFERFKELAPFGIKVGISTGDLDSRADWLGVNDIIVATSEKTDSLLRNGTSWMDEITTVVVDEIHLLDSKNRGPTLEVTITKLMRLNPDVQVVALSATVGNAREMADWLGAALVLSEWRPTDLHEGVLFGDAINFPGSQKKIDRLEKDDAVNLVLDTIKAEGQCLVFESSRRNCAGFAKTASSKVAKILDNDIMIKLAGIAEEVESTGETDTAIVLANCIRKGVAFHHAGLNSNHRKLVENGFRQNLIKVISSTPTLAAGLNLPARRVIIRSYRRFDSNFGMQPIPVLEYKQMAGRAGRPHLDPYGESVLLAKTYDEFAQLMENYVEADAEDIWSKLGTENALRTHVLSTIVNGFASTRQELFDFFGATFFAYQQDKWMLEEVINDCLEFLIDKAMVSETEDIEDASKLFLRGTRLGSLVSMLYIDPLSGSKIVDGFKDIGKSTGGNMGSLEDDKGDDITVTDMTLLHLVCSTPDMRQLYLRNTDYTIVNEYIVAHSDEFHEIPDKLKETDYEWFMGEVKTAMLLEEWVTEVSAEDITRHFNVGEGDIHALADTSEWLMHAAAKLAELLGVEYSSHAYSLEKRIRYGSGLDLMELVGIRGVGRVRARKLYNAGFVSVAKLKGADISVLSKLVGPKVAYNILSGIGVRVNDKHFNSAPISSNTLDTLLDKNQKTFNDFQ

SEQ ID NO: 11 - protein, 8 aa, synthetic construct
  REGION 1..8: note = Exemplary Hel308 motif
QMAGRAGR

SEQ ID NO: 12 - protein, 9 aa, synthetic construct
  REGION 1..9: note = Exemplary extended Hel308 motif
QMAGRAGRP

SEQ ID NO: 13 - protein, 707 aa, unidentified
  source 1..707: note = Cenarchaeum symbiosum
MRISELDIPRPAIEFLEGEGYKKLYPPQAAAAKAGLTDGKSVLVSAPTASGKTLIAAIAMISHLSRNRGKAVYLSPLRALAAEKFAEFGKIGGIPLGRPVRVGVSTGDFEKAGRSLGNNDILVLTNERMDSLIRRRPDWMDEVGLVIADEIHLIGDRSRGPTLEMVLTKLRGLRSSPQVVALSATISNADEIAGWLDCTLVHSTWRPVPLSEGVYQDGEVAMGDGSRHEVAATGGGPAVDLAAESVAEGGQSLIFADTRARSASLAAKASAVIPEAKGADAAKLAAAAKKIISSGGETKLAKTLAELVEKGAAFHHAGLNQDCRSVVEEEFRSGRIRLLASTPTLAAGVNLPARRVVISSVMRYNSSSGMSEPISILEYKQLCGRAGRPQYDKSGEAIVVGGVNADEIFDRYIGGEPEPIRSAMVDDRALRIHVLSLVTTSPGIKEDDVTEFFLGTLGGQQSGESTVKFSVAVALRFLQEEGMLGRRGGRLAATKMGRLVSRLYMDPMTAVTLRDAVGEASPGRMHTLGFLHLVSECSEFMPRFALRQKDHEVAEMMLEAGRGELLRPVYSYECGRGLLALHRWIGESPEAKLAEDLKFESGDVHRMVESSGWLLRCIWEISKHQERPDLLGELDVLRSRVAYGIKAELVPLVSIKGIGRVRSRRLFRGGIKGPGDLAAVPVERLSRVEGIGATLANNIKSQLRKGG

SEQ ID NO: 14 - protein, 8 aa, synthetic construct
  REGION 1..8: note = Exemplary Hel308 motif
QLCGRAGR

SEQ ID NO: 15 - protein, 9 aa, synthetic construct
  REGION 1..9: note = Exemplary extended Hel308 motif
QLCGRAGRP

SEQ ID NO: 16 - protein, 720 aa, Thermococcus gammatolerans
MKVDELPVDERLKAVLKERGIEELYPPQAEALKSGALEGRNLVLAIPTASGKTLVSEIVMVNKLIQEGGKAVYLVPLKALAEEKYREFKEWEKLGLKVAATTGDYDSTDDWLGRYDIIVATAEKFDSLLRHGARWINDVKLVVADEVHLIGSYDRGATLEMILTHMLGRAQILALSATVGNAEELAEWLDASLVVSDWRPVQLRRGVFHLGTLIWEDGKVESYPENWYSLVVDAVKRGKGALVFVNTRRSAEKEALALSKLVSSHLTKPEKRALESLASQLEDNPTSEKLKRALRGGVAFHHAGLSRVERTLIEDAFREGLIKVITATPTLSAGVNLPSFRVIIRDTKRYAGFGWTDIPVLEIQQMMGRAGRPRYDKYGEAIIVARTDEPGKLMERYIRGKPEKLFSMLANEQAFRSQVLALITNFGIRSFPELVRFLERTFYAHQRKDLSSLEYKAKEVVYFLIENEFIDLDLEDRFIPLPFGKRTSQLYIDPLTAKKFKDAFPAIERNPNPFGIFQLIASTPDMATLTARRREMEDYLDLAYELEDKLYASIPYYEDSRFQGFLGQVKTAKVLLDWINEVPEARIYETYSIDPGDLYRLLELADWLMYSLIELYKLFEPKEEILNYLRDLHLRLRHGVREELLELVRLPNIGRKRARALYNAGFRSVEAIANAKPAELLAVEGIGAKILDGIYRHLGIEKRVTEEKPKRKGTLEDFLR

SEQ ID NO: 17 - protein, 8 aa, synthetic construct
  REGION 1..8: note = Exemplary extended Hel308 motif
QMMGRAGR

SEQ ID NO: 18 - protein, 9 aa, synthetic construct
  REGION 1..9: note = Exemplary extended Hel308 motif
QMMGRAGRP

SEQ ID NO: 19 - protein, 799 aa, Methanospirillum hungatei
MEIASLPLPDSFIRACHAKGIRSLYPPQAECIEKGLLEGKNLLISIPTASGKTLLAEMAMWSRIAAGGKCLYIVPLRALASEKYDEFSKKGVIRVGIATGDLDRTDAYLGENDIIVATSEKTDSLLRNRTPWLSQITCIVLDEVHLIGSENRGATLEMVITKLRYTNPVMQIIGLSATIGNPAQLAEWLDATLITSTWRPVDLRQGVYYNGKIRFSDSERPIQGKTKHDDLNLCLDTIEEGGQCLVFVSSRRNAEGFAKKAAGALKAGSPDSKALAQELRRLRDRDEGNVLADCVERGAAFHHAGLIRQERTIIEEGFRNGYIEVIAATPTLAAGLNLPARRVIIRDYNRFASGLGMVPIPVGEYHQMAGRAGRPHLDPYGEAVLLAKDAPSVERLFETFIDAEAERVDSQCVDDASLCAHILSLIATGFAHDQEALSSFMERTFYFFQHPKTRSLPRLVADAIRFLTTAGMVEERENTLSATRLGSLVSRLYLNPCTARLILDSLKSCKTPTLIGLLHVICVSPDMQRLYLKAADTQLLRTFLFKHKDDLILPLPFEQEEEELWLSGLKTALVLTDWADEFSEGMIEERYGIGAGDLYNIVDSGKWLLHGTERLVSVEMPEMSQVVKTLSVRVHHGVKSELLPLVALRNIGRVRARTLYNAGYPDPEAVARAGLSTIARIIGEGIARQVIDEITGVKRSGIHSSDDDYQQKTPELLTDIPGIGKKMAEKLQNAGIITVSDLLTADEVLLSDVLGAARARKVLAFLSNSEKENSSSDKTEEIPDTQKIRGQSSWEDFGC

SEQ ID NO: 20-23 - skipped (4 entries)

SEQ ID NO: 24 - protein, 8 aa, synthetic construct
  REGION 1..8: note = RecD motif I
  VARIANT 5: note = X = T, V or C
  VARIANT 8: note = X = T or S
GGPGXGKX

SEQ ID NO: 25 - protein, 8 aa, synthetic construct
  REGION 1..8: note = Preferred RecD motif I
GGPGTGKT

SEQ ID NO: 26 - protein, 25 aa, synthetic construct
  REGION 1..25: note = Extended RecD motif I
  VARIANT 2..17: note = X = any amino acid
  VARIANT 22: note = X = T, V or C
  VARIANT 25: note = X = T or S
QXXXXXXXXXXXXXXXXGGPGXGKX

SEQ ID NO: 27 - protein, 26 aa, synthetic construct
  REGION 1..26: note = Extended RecD motif I
  VARIANT 2..18: note = X = any amino acid
  VARIANT 23: note = X = T, V or C
  VARIANT 26: note = X = T or S
QXXXXXXXXXXXXXXXXXGGPGXGKX

SEQ ID NO: 28 - protein, 27 aa, synthetic construct
  REGION 1..27: note = Extended RecD motif I
  VARIANT 2..19: note = X = any amino acid
  VARIANT 24: note = X = T, V or C
  VARIANT 27: note = X = T or S
QXXXXXXXXXXXXXXXXXXGGPGXGKX

SEQ ID NO: 29 - skipped

SEQ ID NO: 30 - protein, 10 aa, synthetic construct
  REGION 1..10: note = RecD motif V
  VARIANT 1: note = X = Y, W or F
  VARIANT 2: note = X = A, M, C or V
  VARIANT 3: note = X = I, M or L
  VARIANT 4: note = X = T or S
  VARIANT 5: note = X = V or I
XXXXXHKSQG

SEQ ID NO: 31 - protein, 13 aa, synthetic construct
  REGION 1..13: note = MobF motif III
  VARIANT 2..3: note = X = any amino acid
  VARIANT 4: note = X = any amino acid except D, E, R and K
  VARIANT 6..10: note = X = any amino acid
  VARIANT 12: note = X = any amino acid except D, E, R and K
HXXXRXXXXXHXH

SEQ ID NO: 32 - protein, 14 aa, synthetic construct
  REGION 1..14: note = MobF motif III
  VARIANT 2..3: note = X = any amino acid
  VARIANT 4: note = X = any amino acid except D, E, R and K
  VARIANT 6..11: note = X = any amino acid
  VARIANT 13: note = X = any amino acid except D, E, R and K
HXXXRXXXXXXHXH

SEQ ID NO: 33 - protein, 15 aa, synthetic construct
  REGION 1..15: note = MobF motif III
  VARIANT 2..3: note = X = any amino acid
  VARIANT 4: note = X = any amino acid except D, E, R and K
  VARIANT 6..12: note = X = any amino acid
  VARIANT 14: note = X = any amino acid except D, E, R and K
HXXXRXXXXXXXHXH

SEQ ID NO: 34 - protein, 16 aa, synthetic construct
  REGION 1..16: note = MobF motif III
  VARIANT 2..3: note = X = any amino acid
  VARIANT 4: note = X = any amino acid except D, E, R and K
  VARIANT 6..13: note = X = any amino acid
  VARIANT 15: note = X = any amino acid except D, E, R and K
HXXXRXXXXXXXXHXH

SEQ ID NO: 35 - protein, 17 aa, synthetic construct
  REGION 1..17: note = MobF motif III
  VARIANT 2..3: note = X = any amino acid
  VARIANT 4: note = X = any amino acid except D, E, R and K
  VARIANT 6..14: note = X = any amino acid
  VARIANT 16: note = X = any amino acid except D, E, R and K
HXXXRXXXXXXXXXHXH

SEQ ID NO: 36 - protein, 18 aa, synthetic construct
  REGION 1..18: note = MobF motif III
  VARIANT 2..3: note = X = any amino acid
  VARIANT 4: note = X = any amino acid except D, E, R and K
  VARIANT 6..15: note = X = any amino acid
  VARIANT 17: note = X = any amino acid except D, E, R and K
HXXXRXXXXXXXXXXHXH

SEQ ID NO: 37 - protein, 19 aa, synthetic construct
  REGION 1..19: note = MobF motif III
  VARIANT 2..3: note = X = any amino acid
  VARIANT 4: note = X = any amino acid except D, E, R and K
  VARIANT 6..16: note = X = any amino acid
  VARIANT 18: note = X = any amino acid except D, E, R and K
HXXXRXXXXXXXXXXXHXH

SEQ ID NO: 38 - protein, 20 aa, synthetic construct
  REGION 1..20: note = MobF motif III
  VARIANT 2..3: note = X = any amino acid
  VARIANT 4: note = X = any amino acid except D, E, R and K
  VARIANT 6..17: note = X = any amino acid
  VARIANT 19: note = X = any amino acid
HXXXRXXXXXXXXXXXXHXH

SEQ ID NO: 39-45 - skipped (7 entries)

SEQ ID NO: 46 - protein, 1756 aa, Escherichia coli
MMSIAQVRSAGSAGNYYTDKDNYYVLGSMGERWAGKGAEQLGLQGSVDKDVFTRLLEGRLPDGADLSRMQDGSNKHRPGYDLTFSAPKSVSMMAMLGGDKRLIDAHNQAVDFAVRQVEALASTRVMTDGQSETVLTGNLVMALFNHDTSRDQEPQLHTHAVVANVTQHNGEWKTLSSDKVGKTGFIENVYANQIAFGRLYREKLKEQVEALGYETEVVGKHGMWEMPGVPVEAFSGRSQAIREAVGEDASLKSRDVAALDTRKSKQHVDPEIRMAEWMQTLKETGFDIRAYRDAADQRTEIRTQAPGPASQDGPDVQQAVTQAIAGLSERKVQFTYTDVLARTVGILPPENGVIERARAGIDEAISREQLIPLDREKGLFTSGIHVLDELSVRALSRDIMKQNRVTVHPEKSVPRTAGYSDAVSVLAQDRPSLAIVSGQGGAAGQRERVAELVMMAREQGREVQIIAADRRSQMNLKQDERLSGELITGRRQLLEGMAFTPGSTVIVDQGEKLSLKETLTLLDGAARHNVQVLITDSGQRTGTGSALMAMKDAGVNTYRWQGGEQRPATIISEPDRNVRYARLAGDFAASVKAGEESVAQVSGVREQAILTQAIRSELKTQGVLGHPEVTMTALSPVWLDSRSRYLRDMYRPGMVMEQWNPETRSHDRYVIDRVTAQSHSLTLRDAQGETQVVRISSLDSSWSLFRPEKMPVADGERLRVTGKIPGLRVSGGDRLQVASVSEDAMTVVVPGRAEPASLPVSDSPFTALKLENGWVETPGHSVSDSATVFASVTQMAMDNATLNGLARSGRDVRLYSSLDETRTAEKLARHPSFTVVSEQIKARAGETLLETAISLQKAGLHTPAQQAIHLALPVLESKNLAFSMVDLLTEAKSFAAEGTGFTELGGEINAQIKRGDLLYVDVAKGYGTGLLVSRASYEAEKSILRHILEGKEAVTPLMERVPGELMETLTSGQRAATRMILETSDRFTVVQGYAGVGKTTQFRAVMSAVNMLPASERPRVVGLGPTHRAVGEMRSAGVDAQTLASFLHDTQLQQRSGETPDFSNTLFLLDESSMVGNTEMARAYALIAAGGGRAVASGDTDQLQAIAPGQSFRLQQTRSAADVVIMKEIVRQTPELREAVYSLINRDVERALSGLESVKPSQVPRLEGAWAPEHSVTEFSHSQEAKLAEAQQKAMLKGEAFPDIPMTLYEAIVRDYTGRTPEAREQTLIVTHLNEDRRVLNSMIHDAREKAGELGKEQVMVPVLNTANIRDGELRRLSTWEKNPDALALVDNVYHRIAGISKDDGLITLQDAEGNTRLISPREAVAEGVTLYTPDKIRVGTGDRMRFTKSDRERGYVANSVWTVTAVSGDSVTLSDGQQTRVIRPGQERAEQHIDLAYAITAHGAQGASETFAIALEGTEGNRKLMAGFESAYVALSRMKQHVQVYTDNRQGWTDAINNAVQKGTAHDVLEPKPDREVMNAQRLFSTARELRDVAAGRAVLRQAGLAGGDSPARFIAPGRKYPQPYVALPAFDRNGKSAGIWLNPLTTDDGNGLRGFSGEGRVKGSGDAQFVALQGSRNGESLLADNMQDGVRIARDNPDSGVVVRIAGEGRPWNPGAITGGRVWGDIPDNSVQPGAGNGEPVTAEVLAQRQAEEAIRRETERRADEIVRKMAENKPDLPDGKTELAVRDIAGQERDRSAISERETALPESVLRESQREREAVREVARENLLQERLQQMERDMVRDLQKEKTLGGD

SEQ ID NO: 47 - protein, 8 aa, synthetic construct
  REGION 1..8: note = RecD-like motif I of TraI Eco
GYAGVGKT

SEQ ID NO: 48 - protein, 10 aa, synthetic construct
  REGION 1..10: note = RecD-like motif V of TraI Eco
YAITAHGAQG

SEQ ID NO: 49 - protein, 14 aa, synthetic construct
  REGION 1..14: note = MobF motif III of TraI Eco
HDTSRDQEPQLHTH

SEQ ID NO: 50 - skipped

SEQ ID NO: 51 - protein, 22 aa, synthetic construct
  REGION 1..22: note = XPD motif VI
  VARIANT 2: note = Any amino acid
  VARIANT 3: note = Any amino acid except D, E, K, R. Typically G, P, A, V, L, I, M, C, F, Y, W, H, Q, N, S or T. Preferably not charged. Preferably not H. More preferably V, A, L, I or M.
  VARIANT 6: note = Any amino acid except D, E, K, R. Typically G, P, A, V, L, I, M, C, F, Y, W, H, Q, N, S or T. Preferably not charged. Preferably not H. More preferably V, A, L, I, M or C.
  VARIANT 7: note = Any amino acid except D, E, K, R. Typically G, P, A, V, L, I, M, C, F, Y, W, H, Q, N, S or T. Preferably not charged. Preferably not H. More preferably I, H, L, F, M or V.
  VARIANT 9..11: note = Any amino acid.
  VARIANT 12: note = D or E
  VARIANT 13: note = Any amino acid.
  VARIANT 14: note = Any amino acid. Preferably G, A, S or C
  VARIANT 15..16: note = Any amino acid.
  VARIANT 17: note = Any amino acid. Preferably F, V, L, I, M, A, W or Y
  VARIANT 18: note = Any amino acid. Preferably L, F, Y, M, I or V.
  VARIANT 19: note = Any amino acid. Preferably A, C, V, L, I, M or S.
QXXGRXXRXXXXXXXXXXXDNR

SEQ ID NO: 52 - protein, 726 aa, Methanococcoides burtonii
MSDKPAFMKYFTQSSCYPNQQEAMDRIHSALMQQQLVLFEGACGTGKTLSALVPALHVGKMLGKTVIIATNVHQQMVQFINEARDIKKVQDVKVAVIKGKTAMCPQEADYEECSVKRENTFELMETEREIYLKRQELNSARDSYKKSHDPAFVTLRDELSKEIDAVEEKARGLRDRACNDLYEVLRSDSEKFREWLYKEVRSPEEINDHAIKDGMCGYELVKRELKHADLLICNYHHVLNPDIFSTVLGWIEKEPQETIVIFDEAHNLESAARSHSSLSLTEHSIEKAITELEANLDLLADDNIHNLFNIFLEVISDTYNSRFKFGERERVRKNWYDIRISDPYERNDIVRGKFLRQAKGDFGEKDDIQILLSEASELGAKLDETYRDQYKKGLSSVMKRSHIRYVADFMSAYIELSHNLNYYPILNVRRDMNDEIYGRVELFTCIPKNVTEPLFNSLFSVILMSATLHPFEMVKKTLGITRDTCEMSYGTSFPEEKRLSIAVSIPPLFAKNRDDRHVTELLEQVLLDSIENSKGNVILFFQSAFEAKRYYSKIEPLVNVPVFLDEVGISSQDVREEFFSIGEENGKAVLLSYLWGTLSEGIDYRDGRGRTVIIIGVGYPALNDRMNAVESAYDHVFGYGAGWEFAIQVPTIRKIRQAMGRVVRSPTDYGARILLDGRFLTDSKKRFGKFSVFEVFPPAERSEFVDVDPEKVKYSLMNFFMDNDEQ

SEQ ID NO: 53 - protein, 9 aa, synthetic construct
  REGION 1..9: note = Motif V
YLWGTLSEG

SEQ ID NO: 54 - protein, 22 aa, synthetic construct
  REGION 1..22: note = Motif VI
QAMGRVVRSPTDYGARILLDGR

SEQ ID NO: 55 - protein, 301 aa, unidentified
  source 1..301: note = Bacteriophage T4
MFKRKSTAELAAQMAKLNGNKGFSSEDKGEWKLKLDNAGNGQAVIRFLPSKNDEQAPFAILVNHGFKKNGKWYIETCSSTHGDYDSCPVCQYISKNDLYNTDNKEYSLVKRKTSYWANILVVKDPAAPENEGKVFKYRFGKKIWDKINAMIAVDVEMGETPVDVTCPWEGANFVLKVKQVSGFSNYDESKFLNQSAIPNIDDESFQKELFEQMVDLSEMTSKDKFKSFEELNTKFGQVMGTAVMGGAAATAAKKADKVADDLDAFNVDDFNTKTEDDFMSSSSGSSSSADDTDLDDLLNDL

SEQ ID NO: 56 - protein, 299 aa, unidentified
  source 1..299: note = Bacteriophage RB69
MFKRKSTADLAAQMAKLNGNKGFSSEDKGEWKLKLDASGNGQAVIRFLPAKTDDALPFAILVNHGFKKNGKWYIETCSSTHGDYDSCPVCQYISKNDLYNTNKTEYSQLKRKTSYWANILVVKDPQAPDNEGKVFKYRFGKKIWDKINAMIAVDTEMGETPVDVTCPWEGANFVLKVKQVSGFSNYDESKFLNQSAIPNIDDESFQKELFEQMVDLSEMTSKDKFKSFEELNTKFNQVLGTAALGGAAAAAASVADKVASDLDDFDKDMEAFSSAKTEDDFMSSSSSDDGDLDDLLAGL

SEQ ID NO: 57 - protein, 232 aa, unidentified
  source 1..232: note = Bacteriophage T7
MAKKIFTSALGTAEPYAYIAKPDYGNEERGFGNPRGVYKVDLTIPNKDPRCQRMVDEIVKCHEEAYAAAVEEYEANPPAVARGKKPLKPYEGDMPFFDNGDGTTTFKFKCYASFQDKKTKETKHINLVVVDSKGKKMEDVPIIGGGSKLKVKYSLVPYKWNTAVGASVKLQLESVMLVELATFGGGEDDWADEVEENGYVASGSAKASKPRDEESWDEDDEESEEADEDGDF

SEQ ID NO: 58 - protein, 608 aa, unidentified
  source 1..608: note = Bacteriophage phi-29
MKHMPRKMYSCAFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKSGGSAWSHPQFEKGGGSGGGSGGSAWSHPQFEK

SEQ ID NO: 59 - protein, 233 aa, unidentified
  source 1..233: note = Bacteriophage RB69
KGFSSEDKGEWKLKLDASGNGQAVIRFLPAKTDDALPFAILVNHGFKKNGKWYIETCSSTHGDYDSCPVCQYISKNDLYNTNKTEYSQLKRKTSYWANILVVKDPQAPDNEGKVFKYRFGKKIWDKINAMIAVDTEMGETPVDVTCPWEGANFVLKVKQVSGFSNYDESKFLNQSAIPNIDDESFQKELFEQMVDLSEMTSKDKFKSFEELNTKFNQVLGTAALGGAAAAAAS

SEQ ID NO: 60 - protein, 210 aa, unidentified
  source 1..210: note = Bacteriophage T7
AKKIFTSALGTAEPYAYIAKPDYGNEERGFGNPRGVYKVDLTIPNKDPRCQRMVDEIVKCHEEAYAAAVEEYEANPPAVARGKKPLKPYEGDMPFFDNGDGTTTFKFKCYASFQDKKTKETKHINLVVVDSKGKKMEDVPIIGGGSKLKVKYSLVPYKWNTAVGASVKLQLESVMLVELATFGGGEDDWADEVEENGYVASGSAKASKPR

SEQ ID NO: 61 - protein, 99 aa, Halorubrum lacusprofundi
SGEELLDLAGVRNVGRKRARRLFEAGIETRADLREADKAVVLGALRGRERTAERILEHAGREDPSMDDVRPDKSASAAATAGSASDEDGEGQASLGDFR

SEQ ID NO: 62 - protein, 102 aa, Haloferax volcanii
SGEELLDLAGVRGVGRKRARRLFEAGVETRADLREADKPRVLAALRGRRKTAENILEAAGRKDPSMDAVDEDDAPDDAVPDDAGFETAKERADQQASLGDFE

SEQ ID NO: 63 - protein, 132 aa, Homo sapiens
ESETTTSLVLERSLNRVHLLGRVGQDPVLRQVEGKNPVTIFSLATNEMWRSGDSEVYQLGDVSQKTTWHRISVFRPGLRDVAYQYVKKGSRIYLEGKIDYGEYMDKNNVRRQATTIIADNIIFLSDQTKEKE

SEQ ID NO: 64 - protein, 123 aa, unidentified
  source 1..123: note = Bacteriophage phi-29
ENTNIVKATFDTETLEGQIKIFNAQTGGGQSFKNLPDGTIIEANAIAQYKQVSDTYGDAKEETVTTIFAADGSLYSAISKTVAEAASDLIDLVTRHKLETFKVKVVQGTSSKGNVFFSLQLSL

SEQ ID NO: 65 - protein, 177 aa, Escherichia coli
ASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMDFDDDIPF

SEQ ID NO: 66 - protein, 177 aa, synthetic construct
  REGION 1..177: note = EcoSSB-CterAla
ASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMAFAAAIPF

SEQ ID NO: 67 - protein, 177 aa, synthetic construct
  REGION 1..177: note = EcoSSB-CterNGGN
ASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMNFGGNIPF

SEQ ID NO: 68 - protein, 152 aa, synthetic construct
  REGION 1..152: note = EcoSSB-Q152del
ASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQ

SEQ ID NO: 69 - protein, 117 aa, synthetic construct
  REGION 1..117: note = EcoSSB-G117del
ASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQG

SEQ ID NO: 70 - DNA, 5206 bp, synthetic construct
  misc_feature 1..5206: note = Polynucleotide used in the Examples
  source 1..5206: mol_type = other DNA
ggttgtttctgttggtgctgatattgcccggtggtaccttacgcgttggatgaggagaagtggcttaatatgcttggcacgttcgtcaaggactggtttagatatgagtcacattttgttcatggtagagattctcttgttgacattttaaaagagcgtggattactatctgagtccgatgctgttcaaccactaataggtaagaaatcatgagtcaagttactgaacaatccgtacgtttccagaccgctttggcctctattaagctcattcaggcttctgccgttttggatttaaccgaagatgatttcgattttctgacgagtaacaaagtttggattgctactgaccgctctcgtgctcgtcgctgcgttgaggcttgcgtttatggtacgctggactttgtaggataccctcgctttcctgctcctgttgagtttattgctgccgtcattgcttattatgttcatcccgtcaacattcaaacggcctgtctcatcatggaaggcgctgaatttacggaaaacattattaatggcgtcgagcgtccggttaaagccgctgaattgttcgcgtttaccttgcgtgtacgcgcaggaaacactgacgttcttactgacgcagaagaaaacgtgcgtcaaaaattacgtgcagaaggagtgatgtaatgtctaaaggtaaaaaacgttctggcgctcgccctggtcgtccgcagccgttgcgaggtactaaaggcaagcgtaaaggcgctcgtctttggtatgtaggtggtcaacaattttaattgcaggggcttcggccccttacttgaggataaattatgtctaatattcaaactggcgccgagcgtatgccgcatgacctttcccatcttggcttccttgctggtcagattggtcgtcttattaccatttcaactactccggttatcgctggcgactccttcgagatggacgccgttggcgctctccgtctttctccattgcgtcgtggccttgctattgactctactgtagacatttttactttttatgtccctcatcgtcacgtttatggtgaacagtggattaagttcatgaaggatggtgttaatgccactcctctcccgactgttaacactactggttatattgaccatgccgcttttcttggcacgattaaccctgataccaataaaatccctaagcatttgtttcagggttatttgaatatctataacaactattttaaagcgccgtggatgcctgaccgtaccgaggctaaccctaatgagcttaatcaagatgatgctcgttatggtttccgttgctgccatctcaaaaacatttggactgctccgcttcctcctgagactgagctttctcgccaaatgacgacttctaccacatctattgacattatgggtctgcaagctgcttatgctaatttgcatactgaccaagaacgtgattacttcatgcagcgttaccatgatgttatttcttcatttggaggtaaaacctcttatgacgctgacaaccgtcctttacttgtcatgcgctctaatctctgggcatctggctatgatgttgatggaactgaccaaacgtcgttaggccagttttctggtcgtgttcaacagacctataaacattctgtgccgcgtttctttgttcctgagcatggcactatgtttactcttgcgcttgttcgttttccgcctactgcgactaaagagattcagtaccttaacgctaaaggtgctttgacttataccgatattgctggcgaccctgttttgtatggcaacttgccgccgcgtgaaatttctatgaaggatgttttccgttctggtgattcgtctaagaagtttaagattgctgagggtcagtggtatcgttatgcgccttcgtatgtttctcctgcttatcaccttcttgaaggcttcccattcattcaggaaccgccttctggtgatttgcaagaacgcgtacttattcgccaccatgattatgaccagtgtttccagtccgttcagttgttgcagtggaatagtcaggttaaatttaatgtgaccgtttatcgcaatctgccgaccactcgcgattcaatcatgacttcgtgataaaagattgagtgtgaggttataacgccgaagcggtaaaaattttaatttttgccgctgaggggttgaccaagcgaagcgcggtaggttttctgcttaggagtttaatcatgtttcagacttttatttctcgccataattcaaactttttttctgataagctggttctcacttctgttactccagcttcttcggcacctgttttacagacacctaaagctacatcgtcaacgttatattttgatagtttgacggttaatgctggtaatggtggttttcttcattgcattcagatggatacatctgtcaacgccgctaatcaggttgtttctgttggtgctgatattgcttttgatgccgaccctaaattttttgcctgtttggttcgctttgagtcttcttcggttccgactaccctcccgactgcctatgatgtttatcctttggatggtcgccatgatggtggttattataccgtcaaggactgtgtgactattgacgtccttccccgtacgccgggcaataatgtttatgttggtttcatggtttggtctaactttaccgctactaaatgccgcggattggtttcgctgaatcaggttattaaagagattatttgtctccagccacttaagtgaggtgatttatgtttggtgctattgctggcggtattgcttctgctcttgctggtggcgccatgtctaaattgtttggaggcggtcaaaaagccgcctccggtggcattcaaggtgatgtgcttgctaccgataacaatactgtaggcatgggtgatgctggtattaaatctgccattcaaggctctaatgttcctaaccctgatgaggccgtccctagttttgtttctggtgctatggctaaagctggtaaaggacttcttgaaggtacgttgcaggctggcacttctgccgtttctgataagttgcttgatttggttggacttggtggcaagtctgccgctgataaaggaaaggatactcgtgattatcttgctgctgcatttcctgagcttaatgcttgggagcgtgctggtgctgatgcttcctctgctggtatggttgacgccggatttgagaatcaaaaagagcttactaaaatgcaactggacaatcagaaagagattgccgagatgcaaaatgagactcaaaaagagattgctggcattcagtcggcgacttcacgccagaatacgaaagaccaggtatatgcacaaaatgagatgcttgcttatcaacagaaggagtctactgctcgcgttgcgtctattatggaaaacaccaatctttccaagcaacagcaggtttccgagattatgcgccaaatgcttactcaagctcaaacggctggtcagtattttaccaatgaccaaatcaaagaaatgactcgcaaggttagtgctgaggttgacttagttcatcagcaaacgcagaatcagcggtatggctcttctcatattggcgctactgcaaaggatatttctaatgtcgtcactgatgctgcttctggtgtggttgatatttttcatggtattgataaagctgttgccgatacttggaacaatttctggaaagacggtaaagctgatggtattggctctaatttgtctaggaaataaccgtcaggattgacaccctcccaattgtatgttttcatgcctccaaatcttggaggcttttttatggttcgttcttattacccttctgaatgtcacgctgattattttgactttgagcgtatcgaggctcttaaacctgctattgaggcttgtggcatttctactctttctcaatccccaatgcttggcttccataagcagatggataaccgcatcaagctcttggaagagattctgtcttttcgtatgcagggcgttgagttcgataatggtgatatgtatgttgacggccataaggctgcttctgacgttcgtgatgagtttgtatctgttactgagaagttaatggatgaattggcacaatgctacaatgtgctcccccaacttgatattaataacactatagaccaccgccccgaaggggacgaaaaatggtttttagagaacgagaagacggttacgcagttttgccgcaagctggctgctgaacgccctcttaaggatattcgcgatgagtataattaccccaaaaagaaaggtattaaggatgagtgttcaagattgctggaggcctccactatgaaatcgcgtagaggctttgctattcagcgtttgatgaatgcaatgcgacaggctcatgctgatggttggtttatcgtttttgacactctcacgttggctgacgaccgattagaggcgttttatgataatcccaatgctttgcgtgactattttcgtgatattggtcgtatggttcttgctgccgagggtcgcaaggctaatgattcacacgccgactgctatcagtatttttgtgtgcctgagtatggtacagctaatggccgtcttcatttccatgcggtgcactttatgcggacacttcctacaggtagcgttgaccctaattttggtcgtcgggtacgcaatcgccgccagttaaatagcttgcaaaatacgtggccttatggttacagtatgcccatcgcagttcgctacacgcaggacgctttttcacgttctggttggttgtggcctgttgatgctaaaggtgagccgcttaaagctaccagttatatggctgttggtttctatgtggctaaatacgttaacaaaaagtcagatatggaccttgctgctaaaggtctaggagctaaagaatggaacaactcactaaaaaccaagctgtcgctacttcccaagaagctgttcagaatcagaatgagccgcaacttcgggatgaaaatgctcacaatgacaaatctgtccacggagtgcttaatccaacttaccaagctgggttacgacgcgacgccgttcaaccagatattgaagcagaacgcaaaaagagagatgagattgaggctgggaaaagttactgtagccgacgttttggcggcgcaacctgtgacgacaaatctgctcaaatttatgcgcgcttcgataaaaatgattggcgtatccaacctgca

SEQ ID NO: 71 - DNA, 5175 bp, synthetic construct
  misc_feature 1..5175: note = Polynucleotide used in the Examples
  source 1..5175: mol_type = other DNA
ccggtggtaccttacgcgttggatgaggagaagtggcttaatatgcttggcacgttcgtcaaggactggtttagatatgagtcacattttgttcatggtagagattctcttgttgacattttaaaagagcgtggattactatctgagtccgatgctgttcaaccactaataggtaagaaatcatgagtcaagttactgaacaatccgtacgtttccagaccgctttggcctctattaagctcattcaggcttctgccgttttggatttaaccgaagatgatttcgattttctgacgagtaacaaagtttggattgctactgaccgctctcgtgctcgtcgctgcgttgaggcttgcgtttatggtacgctggactttgtaggataccctcgctttcctgctcctgttgagtttattgctgccgtcattgcttattatgttcatcccgtcaacattcaaacggcctgtctcatcatggaaggcgctgaatttacggaaaacattattaatggcgtcgagcgtccggttaaagccgctgaattgttcgcgtttaccttgcgtgtacgcgcaggaaacactgacgttcttactgacgcagaagaaaacgtgcgtcaaaaattacgtgcagaaggagtgatgtaatgtctaaaggtaaaaaacgttctggcgctcgccctggtcgtccgcagccgttgcgaggtactaaaggcaagcgtaaaggcgctcgtctttggtatgtaggtggtcaacaattttaattgcaggggcttcggccccttacttgaggataaattatgtctaatattcaaactggcgccgagcgtatgccgcatgacctttcccatcttggcttccttgctggtcagattggtcgtcttattaccatttcaactactccggttatcgctggcgactccttcgagatggacgccgttggcgctctccgtctttctccattgcgtcgtggccttgctattgactctactgtagacatttttactttttatgtccctcatcgtcacgtttatggtgaacagtggattaagttcatgaaggatggtgttaatgccactcctctcccgactgttaacactactggttatattgaccatgccgcttttcttggcacgattaaccctgataccaataaaatccctaagcatttgtttcagggttatttgaatatctataacaactattttaaagcgccgtggatgcctgaccgtaccgaggctaaccctaatgagcttaatcaagatgatgctcgttatggtttccgttgctgccatctcaaaaacatttggactgctccgcttcctcctgagactgagctttctcgccaaatgacgacttctaccacatctattgacattatgggtctgcaagctgcttatgctaatttgcatactgaccaagaacgtgattacttcatgcagcgttaccatgatgttatttcttcatttggaggtaaaacctcttatgacgctgacaaccgtcctttacttgtcatgcgctctaatctctgggcatctggctatgatgttgatggaactgaccaaacgtcgttaggccagttttctggtcgtgttcaacagacctataaacattctgtgccgcgtttctttgttcctgagcatggcactatgtttactcttgcgcttgttcgttttccgcctactgcgactaaagagattcagtaccttaacgctaaaggtgctttgacttataccgatattgctggcgaccctgttttgtatggcaacttgccgccgcgtgaaatttctatgaaggatgttttccgttctggtgattcgtctaagaagtttaagattgctgagggtcagtggtatcgttatgcgccttcgtatgtttctcctgcttatcaccttcttgaaggcttcccattcattcaggaaccgccttctggtgatttgcaagaacgcgtacttattcgccaccatgattatgaccagtgtttccagtccgttcagttgttgcagtggaatagtcaggttaaatttaatgtgaccgtttatcgcaatctgccgaccactcgcgattcaatcatgacttcgtgataaaagattgagtgtgaggttataacgccgaagcggtaaaaattttaatttttgccgctgaggggttgaccaagcgaagcgcggtaggttttctgcttaggagtttaatcatgtttcagacttttatttctcgccataattcaaactttttttctgataagctggttctcacttctgttactccagcttcttcggcacctgttttacagacacctaaagctacatcgtcaacgttatattttgatagtttgacggttaatgctggtaatggtggttttcttcattgcattcagatggatacatctgtcaacgccgctaatcaggttgtttctgttggtgctgatattgcttttgatgccgaccctaaattttttgcctgtttggttcgctttgagtcttcttcggttccgactaccctcccgactgcctatgatgtttatcctttggatggtcgccatgatggtggttattataccgtcaaggactgtgtgactattgacgtccttccccgtacgccgggcaataatgtttatgttggtttcatggtttggtctaactttaccgctactaaatgccgcggattggtttcgctgaatcaggttattaaagagattatttgtctccagccacttaagtgaggtgatttatgtttggtgctattgctggcggtattgcttctgctcttgctggtggcgccatgtctaaattgtttggaggcggtcaaaaagccgcctccggtggcattcaaggtgatgtgcttgctaccgataacaatactgtaggcatgggtgatgctggtattaaatctgccattcaaggctctaatgttcctaaccctgatgaggccgtccctagttttgtttctggtgctatggctaaagctggtaaaggacttcttgaaggtacgttgcaggctggcacttctgccgtttctgataagttgcttgatttggttggacttggtggcaagtctgccgctgataaaggaaaggatactcgtgattatcttgctgctgcatttcctgagcttaatgcttgggagcgtgctggtgctgatgcttcctctgctggtatggttgacgccggatttgagaatcaaaaagagcttactaaaatgcaactggacaatcagaaagagattgccgagatgcaaaatgagactcaaaaagagattgctggcattcagtcggcgacttcacgccagaatacgaaagaccaggtatatgcacaaaatgagatgcttgcttatcaacagaaggagtctactgctcgcgttgcgtctattatggaaaacaccaatctttccaagcaacagcaggtttccgagattatgcgccaaatgcttactcaagctcaaacggctggtcagtattttaccaatgaccaaatcaaagaaatgactcgcaaggttagtgctgaggttgacttagttcatcagcaaacgcagaatcagcggtatggctcttctcatattggcgctactgcaaaggatatttctaatgtcgtcactgatgctgcttctggtgtggttgatatttttcatggtattgataaagctgttgccgatacttggaacaatttctggaaagacggtaaagctgatggtattggctctaatttgtctaggaaataaccgtcaggattgacaccctcccaattgtatgttttcatgcctccaaatcttggaggcttttttatggttcgttcttattacccttctgaatgtcacgctgattattttgactttgagcgtatcgaggctcttaaacctgctattgaggcttgtggcatttctactctttctcaatccccaatgcttggcttccataagcagatggataaccgcatcaagctcttggaagagattctgtcttttcgtatgcagggcgttgagttcgataatggtgatatgtatgttgacggccataaggctgcttctgacgttcgtgatgagtttgtatctgttactgagaagttaatggatgaattggcacaatgctacaatgtgctcccccaacttgatattaataacactatagaccaccgccccgaaggggacgaaaaatggtttttagagaacgagaagacggttacgcagttttgccgcaagctggctgctgaacgccctcttaaggatattcgcgatgagtataattaccccaaaaagaaaggtattaaggatgagtgttcaagattgctggaggcctccactatgaaatcgcgtagaggctttgctattcagcgtttgatgaatgcaatgcgacaggctcatgctgatggttggtttatcgtttttgacactctcacgttggctgacgaccgattagaggcgttttatgataatcccaatgctttgcgtgactattttcgtgatattggtcgtatggttcttgctgccgagggtcgcaaggctaatgattcacacgccgactgctatcagtatttttgtgtgcctgagtatggtacagctaatggccgtcttcatttccatgcggtgcactttatgcggacacttcctacaggtagcgttgaccctaattttggtcgtcgggtacgcaatcgccgccagttaaatagcttgcaaaatacgtggccttatggttacagtatgcccatcgcagttcgctacacgcaggacgctttttcacgttctggttggttgtggcctgttgatgctaaaggtgagccgcttaaagctaccagttatatggctgttggtttctatgtggctaaatacgttaacaaaaagtcagatatggaccttgctgctaaaggtctaggagctaaagaatggaacaactcactaaaaaccaagctgtcgctacttcccaagaagctgttcagaatcagaatgagccgcaacttcgggatgaaaatgctcacaatgacaaatctgtccacggagtgcttaatccaacttaccaagctgggttacgacgcgacgccgttcaaccagatattgaagcagaacgcaaaaagagagatgagattgaggctgggaaaagttactgtagccgacgttttggcggcgcaacctgtgacgacaaatctgctcaaatttatgcgcgcttcgataaaaatgattggcgtatccaacc

SEQ ID NO: 72 - DNA, 29 bp, synthetic construct
  misc_feature 1..29: note = Polynucleotide used in the Examples
  source 1..29: mol_type = other DNA
gcaatatcagcaccaacagaaacaacctt

SEQ ID NO: 73 - DNA, 88 bp, synthetic construct
  misc_feature 1..88: note = Polynucleotide used in the Examples
  source 1..88: mol_type = other DNA
cgtggtcacgaggagctcgtcctcacctcgacgtctgcacgagctttttttttttttttttttttttttttttttttttttttttttt

SEQ ID NO: 74 - protein, 55 aa, synthetic construct
  REGION 1..55: note = (HhH)2 domain
WKEWLERKVGEGRARRLIEYFGSAGEVGKLVENAEVSKLLEVPGIGDEAVARLVP

SEQ ID NO: 75 - protein, 107 aa, synthetic construct
  REGION 1..107: note = (HhH)2-(HhH)2 domain
WKEWLERKVGEGRARRLIEYFGSAGEVGKLVENAEVSKLLEVPGIGDEAVARLVPGYKTLRDAGLTPAEAERVLKRYGSVSKVQEGATPDELRELGLGDAKIARILG

SEQ ID NO: 76 - protein, 318 aa, unidentified
  source 1..318: note = Herpes simplex virus 1
TDSPGGVAPASPVEDASDASLGQPEEGAPCQVVLQGAELNGILQAFAPLRTSLLDSLLVMGDRGILIHNTIFGEQVFLPLEHSQFSRYRWRGPTAAFLSLVDQKRSLLSVFRANQYPDLRRVELAITGQAPFRTLVQRIWTTTSDGEAVELASETLMKRELTSFVVLVPQGTPDVQLRLTRPQLTKVLNATGADSATPTTFELGVNGKFSVFTTSTCVTFAAREEGVSSSTSTQVQILSNALTKAGQAAANAKTVYGENTHRTFSVVVDDCSMRAVLRRLQVGGGTLKFFLTTPVPSLCVTATGPNAVSAVFLLKPQK

SEQ ID NO: 77 - protein, 293 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 78 - DNA, 15 bp, synthetic construct
  misc_feature 1..15: note = Polynucleotide used in the Examples
  source 1..15: mol_type = other DNA
  modified_base order(3,12): mod_base = OTHER; note = uracil
cctagtctccgtagc

SEQ ID NO: 79 - DNA, 354 bp, synthetic construct
  misc_feature 1..354: note = Polynucleotide used in the Examples
  source 1..354: mol_type = other DNA
aggttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttt

SEQ ID NO: 80 - protein, 508 aa, synthetic construct
  REGION 1..508: note = EcoExoI with all natural cysteines removed, A83C and two Strep tags
MMNDGKQQSTFLFHDYETFGTHPALDRPAQFAAIRTDSEFNVIGEPEVFYAKPADDYLPQPGAVLITGITPQEARAKGENEACFAARIHSLFTVPKTTILGYNNVRFDDEVTRNIFYRNFYDPYAWSWQHDNSRWDLLDVMRATYALRPEGINWPENDDGLPSFRLEHLTKANGIEHSNAHDAMADVYATIAMAKLVKTRQPRLFDYLFTHRNKHKLMALIDVPQMKPLVHVSGMFGAWRGNTSWVAPLAWHPENRNAVIMVDLAGDISPLLELDSDTLRERLYTAKTDLGDNAAVPVKLVHINKTPVLAQANTLRPEDADRLGINRQHTLDNLKILRENPQVREKVVAIFAEAEPFTPSDNVDAQLYNGFFSDADRAAMKIVLETEPRNLPALDITFVDKRIEKLLFNYRARNFPGTLDYAEQQRWLEHRRQVFTPEFLQGYADELQMLVQQYADDKEKVALLKALWQYAEEIVSGGSAWSHPQFEKGGGSGGGSGGSAWSHPQFEK

SEQ ID NO: 81 - DNA, 89 bp, synthetic construct
  misc_feature 1..89: note = Polynucleotide used in the Examples
  source 1..89: mol_type = other DNA
aggtttttttttttttttttttttttttttttttttttttttttttttttttttttttttttttttttttttttttttttttttttttt

SEQ ID NO: 82 - protein, 608 aa, synthetic construct
  REGION 1..608: note = PhiE T373C/C22A/C455A/C503A-STrEP
MKHMPRKMYSCDFETTTKVEDARVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTCSEGAIKQLAKLMLNSLYGKFASNPDVTGKVPYLKENGALGFRLGEEETKDPVYTPMGVFITAWARYTTITAAQACYDRIIYADTDSIHLTGTEIPDVIKDIVDPKKLGYWAHESTFKRAKYLRQKTYIQDIYMKEVDGKLVEGSPDDYTDIKFSVKAAGMTDKIKKEVTFENFKVGFSRKMKPKPVQVPGGVVLVDDTFTIKSGGSAWSHPQFEKGGGSGGGSGGSAWSHPQFEK

SEQ ID NO: 83 - DNA, 70 bp, synthetic construct
  misc_feature 1..70: note = Polynucleotide used in the Examples
  source 1..70: mol_type = other DNA
tttttttttttttttttttttttttttttttttttttttttttttttttttttttttttttttttttttt

SEQ ID NO: 84 - protein, 6 aa, synthetic construct
  REGION 1..6: note = Linker
GTGSGA

SEQ ID NO: 85 - protein, 6 aa, synthetic construct
  REGION 1..6: note = Linker
GTGSGT

SEQ ID NO: 86 - protein, 970 aa, unidentified
  source 1..970: note = Citromicrobium bathyomarinum JL354
MLSVANVRSPSAAASYFASDNYYASADADRSGQWIGDGAKRLGLEGKVEARAFDALLRGELPDGSSVGNPGQAHRPGTDLTFSVPKSWSLLALVGKDERIIAAYREAVVEALHWAEKNAAETRVVEKGMVVTQATGNLAIGLFQHDTNRNQEPNLHFHAVIANVTQGKDGKWRTLKNDRLWQLNTTLNSIAMARFRVAVEKLGYEPGPVLKHGNFEARGISREQVMAFSTRRKEVLEARRGPGLDAGRIAALDTRASKEGIEDRATLSKQWSEAAQSIGLDLKPLVDRARTKALGQGMEATRIGSLVERGRAWLSRFAAHVRGDPADPLVPPSVLKQDRQTIAAAQAVASAVRHLSQREAAFERTALYKAALDFGLPTTIADVEKRTRALVRSGDLIAGKGEHKGWLASRDAVVTEQRILSEVAAGKGDSSPAITPQKAAASVQAAALTGQGFRLNEGQLAAARLILISKDRTIAVQGIAGAGKSSVLKPVAEVLRDEGHPVIGLAIQNTLVQMLERDTGIGSQTLARFLGGWNKLLDDPGNVALRAEAQASLKDHVLVLDEASMVSNEDKEKLVRLANLAGVHRLVLIGDRKQLGAVDAGKPFALLQRAGIARAEMATNLRARDPVVREAQAAAQAGDVRKALRHLKSHTVEARGDGAQVAAETWLALDKETRARTSIYASGRAIRSAVNAAVQQGLLASREIGPAKMKLEVLDRVNTTREELRHLPAYRAGRVLEVSRKQQALGLFIGEYRVIGQDRKGKLVEVEDKRGKRFRFDPARIRAGKGDDNLTLLEPRKLEIHEGDRIRWTRNDHRRGLFNADQARVVEIANGKVTFETSKGDLVELKKDDPMLKRIDLAYALNVHMAQGLTSDRGIAVMDSRERNLSNQKTFLVTVTRLRDHLTLVVDSADKLGAAVARNKGEKASAIEVTGSVKPTATKGSGVDQPKSVEANKAEKELTRSKSKTLDFGI

SEQ ID NO: 87 - protein, 8 aa, synthetic construct
  REGION 1..8: note = RecD-like motif I of TrwC Cba
GIAGAGKS

SEQ ID NO: 88 - protein, 10 aa, synthetic construct
  REGION 1..10: note = RecD-like motif V of TrwC Cba
YALNVHMAQG

SEQ ID NO: 89 - protein, 14 aa, synthetic construct
  REGION 1..14: note = MobF motif III of TrwC Cba
HDTNRNQEPNLHFH

SEQ ID NO: 90 - protein, 943 aa, unidentified
  source 1..943: note = Halothiobacillus neapolitanus c2
MLRIKNLKGDPSAIIDYAENKKNHPDQKSGYYDAKGAPSAWGGALAADLGLSGSVQAADLKKLLSGELSDGTRFAKEDPDRRLGIDMSFSAPKSVSLAALVGGDERIIQAHDAAVRTAMSMIEQEYATARFGHAGRNVVCSGKLVYAAYRHEDARTVDDIADPQLHTHCIVSNITIDPETGKPRSIDFAWGQDGIKLAGAMYRAELARRLKEMGYELRKSEEGFELAQISDEQVETFSRRRVQVDQALEQQGTDREHASSELKTAVTLATRQGKAQLSAEDQYEEWQQRAAEAELDLSQPVGPRVSVTPPEIDLDHTFEHLSERASVINKDAVRLDALINHMSEGATLSTVDKAIQGAAVTGDVFEIEDGIKRKIITRETLKREQQILLLAQQGRGVNSVLIGVGDTKHLIEDAEQAQGFRFSEGQRRAINLTATTTDQVSGIVGAAGAGKTTAMKTVADLAKSQGLTVVGIAPSSAAADELKSAGADDTMTLATFNLKGEAAGPRLLILDEAGMVSARDGEALLKKLGKEDRLIFVGDPKQLAAVEAGSPFAQLMRSGAIQYAEITEINRQKDQKLLDIAQHFAKGKAEEAVALATKYVTEVPVTLPDKPEHKITRQAKTEARRLAIASATAKRYLELSQEERATTLVLSGTNAVRKQVNEQVRKGLIDKGEINGESFTVSTLDKADMTRAKMRKAGNYKPGQVIKTAGKQAEQSEVVAVNLDQNLIQVKLSDGTLKSIDASRFDVKKTQVFNPRQIDIAAGDKIIFTNNDQATETKNNQIGLIEEIKDGKAIINSNGAKVEIDIQRKLHIDHAYCITIHRSQGQTVDSVIVAGEASRTTTAEAAYVACTRERYKLEIITDNTERLSKNWVRYADRQTAAEALKSSEEKYPHLDEIREELRRELQQELERQEPTNITPELEIEMERSMFDQYTLHSRQPRSY

SEQ ID NO: 91 - protein, 8 aa, synthetic construct
  REGION 1..8: note = RecD-like motif I of TrwC Hne
GAAGAGKT

SEQ ID NO: 92 - protein, 10 aa, synthetic construct
  REGION 1..10: note = RecD-like motif V of TrwC Hne
YCITIHRSQG

SEQ ID NO: 93 - protein, 18 aa, synthetic construct
  REGION 1..18: note = MobF motif III of TrwC Hne
HEDARTVDDIADPQLHTH

SEQ ID NO: 94 - protein, 960 aa, unidentified
  source 1..960: note = Erythrobacter litoralis HTCC2594
MLSVANVRSPTAAASYFASDNYYASADADRSGQWIGGGAKRLGLEGKVEAKAFDALLRGELPDGSSVGNPGQAHRPGTDLSFSVPKSWSLLALVGKDERIIAAYREAVVEALQWAEKNAAETRIVEKGKMVTQATGNLAVGLFQHDTNRNQEPNLHFHAVIANVTQGKDGKWRTLKNDRLWQLNTTLNSIAMARFRVAVEKLGYEPGPVLKHGNFEARGISREQIMAFSTRRKEVLEARRGPGLEAGRIAALDTRASKEEIEDRATLGKQWSETAQSIGLDLTPLVDRARTNALGQSMEATRIGSLVERGRAWLSRFAAHVRGDPADPLVPPSVLKQDRQTIAAAQAVASAIRHLSQREAAFERTALYKAALDFGLPATIADVEKRTRALVRSGDLISGKGEHKGWLASREAVVTEQRILSEVAAGKGNSSPAIEPQKAAASVQAAAATGQGFRLNEGQLAAAELILTSKDRTIAIQGIAGAGKSSVLKPVAEVLRDEGHPVIGLAIQNTLVQMLERETGIGSQTLARFLRGWTKLLGDPGNVALRTEAQASLKDHVLVLDEASMVSNEDKEKLVRLANLAGVHRLVLIGDRKQLGAVDAGKPFALLQRAGIARAEMATNLRARDPVVREAQASAQAGDVRNALRHLKSHTVEAKGDGAQVAAETWLALDKETRARTSIYASGRAIRSAVNAAVQQGLLANREIGPGMMKLDVLDRVNATREELRHLPAYRAGQVLEISRKQQALGLSVGEYRVLGQDRKGRLVEVEDKRGKRFRFDPARIKAGKGDENLTLLEPRKLEIHEGDRIRWTRNDHRRGLFNADQARVVAIAGGKITFETSQGDQVELKRDDPMLKRIDLAYALNAHMAQGLTSDRGIAVMTSSERNLSNQKTFMVTVTRLRDHLTLVVDNAEKLGAAVARNKGEKASALEVTGSVKSTAAKGSGVDQLKPEEANKAEKELTR

SEQ ID NO: 95 - protein, 10 aa, synthetic construct
  REGION 1..10: note = RecD-like motif V of TrwC Eli
YALNAHMAQG

(19) United States
(12) Patent Application Publication
(10) Pub. No.: US 2026/0098293 A1
(43) Pub. Date: Apr. 9, 2026

(54) SSB METHOD

(71) Applicant: Oxford Nanopore Technologies PLC, Oxford (GB)

(72) Inventors: James White, Oxford (GB); Ruth Moysey, Oxford (GB); Mihaela Misca, Oxford (GB)

(73) Assignee: Oxford Nanopore Technologies PLC, Oxford (GB)

(21) Appl. No.: 19/267,814

(22) Filed: Jul. 14, 2025

Related U.S. Application Data
(63) Continuation of application No. 17/481,374, filed on Sep. 22, 2021, now Pat. No. 12,448,646.
Application No. 17/481,374 is a continuation of application No. 14/415,459, now Pat. No. 11,155,860.
(60) Provisional application No. 61/673,457, filed on Jul. 19, 2012.
(60) Provisional application No. 61/774,688, filed on Mar. 8, 2013.

(51) Int. Cl. C12Q 1/6869

(52) U.S. Cl. CPC C12Q 1/6869

(57) ABSTRACT

The invention relates to a method of characterising a target polynucleotide using a single-stranded binding protein (SSB). The SSB is either an SSB comprising a carboxy-terminal (C-terminal) region which does not have a net negative charge or a modified SSB comprising one or more modifications in its C-terminal region which decreases the net negative charge of the C-terminal region.

### FIELD OF THE INVENTION

The invention relates to a method of characterising a target polynucleotide using a single-stranded binding protein (SSB). The SSB is either an SSB comprising a carboxy-terminal (C-terminal) region which does not have a net negative charge or a modified SSB comprising one or more modifications in its C-terminal region which decreases the net negative charge of the C-terminal region.

### BACKGROUND OF THE INVENTION

There is currently a need for rapid and cheap polynucleotide (e.g. DNA or RNA) sequencing and identification technologies across a wide range of applications. Existing technologies are slow and expensive mainly because they rely on amplification techniques to produce large volumes of polynucleotide and require a high quantity of specialist fluorescent chemicals for signal detection.

Transmembrane pores (nanopores) have great potential as direct, electrical biosensors for polymers and a variety of small molecules. In particular, recent focus has been given to nanopores as a potential DNA sequencing technology.

When a potential is applied across a nanopore, there is a change in the current flow when an analyte, such as a nucleotide, resides transiently in the barrel for a certain period of time. Nanopore detection of the nucleotide gives a current change of known signature and duration. In the strand sequencing method, a single polynucleotide strand is passed through the pore and the identity of the nucleotides are derived. Strand sequencing can involve the use of a nucleotide handling protein to control the movement of the polynucleotide through the pore.

### SUMMARY OF THE INVENTION

The inventors have surprisingly demonstrated that certain SSBs may be used, for example, to prevent a target polynucleotide from forming secondary structure or as a molecular brake when the polynucleotide is characterized, such as sequenced, using a transmembrane pore. In particular, the inventors have surprisingly demonstrated that SSBs which lack a negatively charged carboxy-terminal (C-terminal) region will bind to a target polynucleotide and prevent secondary structure formation or act as a molecular brake without blocking the transmembrane pore. The absence of pore block is advantageous because it allows the polynucleotide to be characterised by measuring the current flowing through the pore as the polynucleotide moves through the pore. For strand sequencing, it is preferred that the pore has a high duty cycle, i.e. the pore has a polynucleotide within it as much as possible and is sequencing as much as possible Pore block by something other than the analyte of interest lowers the duty cycle and so also lowers data output. Hence, an absence of pore block helps to maintain a high duty cycle and a high data output. Pore block could also happen when a polynucleotide strand is present in the pore and thus attenuate sequencing.

Pore block can be transient (i.e. the block reverses itself during the experiment) or permanent (i.e. the block is maintained for the duration of the experiment without some sort of intervention). If the block is permanent, then a change in potential may be needed to clear the block. This can be problematic, especially for a sequencing array. If each electrode in the array is not individually addressable, it would be necessary to change the potential in all channels to clear the block in one channel or a few channels. This would of course interrupt any sequencing using the array Δn absence of pore block therefore helps sequencing arrays to function effectively.

Accordingly, the invention provides a method of characterising a target polynucleotide, comprising:
a) contacting the target polynucleotide with a transmembrane pore and a single-stranded binding protein (SSB) such that the target polynucleotide moves through the pore and the SSB does not move through the pore, wherein the SSB is (i) an SSB comprising a carboxy-terminal (C-terminal) region which does not have a net negative charge or (ii) a modified SSB comprising one or more modifications in its C-terminal region which decreases the net negative charge of the C-terminal region; and
b) taking one or more measurements as the polynucleotide moves with respect to the pore wherein the measurements are indicative of one or more characteristics of the target polynucleotide and thereby characterising the target polynucleotide.

The invention also provides:
a method of forming a sensor for characterising a target polynucleotide, comprising forming a complex between a pore and an SSB as defined above and thereby forming a sensor for characterising the target polynucleotide;
a sensor for characterising a target polynucleotide, comprising a complex between (a) a pore and (b) a SSB as defined above;
use of a SSB as defined above in the characterisation of a target polynucleotide using a transmembrane pore;
a kit for characterising a target polynucleotide comprising (a) a transmembrane pore and (b) a SSB as defined above;
an apparatus for characterising target polynucleotides in a sample, comprising (a) a plurality of transmembrane pores and (b) a plurality of SSBs as defined above;
a construct comprising at least one helicase and an SSB as defined above, wherein the helicase is attached to the SSB and the construct has the ability to control the movement of a polynucleotide; and
a method of forming a construct of the invention, comprising attaching an SSB as defined above to at least one helicase and thereby producing a construct of the invention.

### DESCRIPTION OF THE FIGURES

FIG. 1 shows an electrophoretic mobility bandshift assay for ssDNA: SSB complexes. Column 1 contains the 70-polyT (SEQ ID NO: 83), column 2 contains commercial EcoSSB-WT (SEQ ID NO: 65), column 3 contains WT-SSB (SEQ ID NO: 65) and column 4 contains EcoSSB-Q152del (SEQ ID NO. 68). It can be seen that the EcoSSB-Q152del mutant (SEQ ID NO. 68) is not impaired in its ability to form a complex with the 70mer polyT (SEQ ID NO: 83), when compared to the wild-type SSB (SEQ ID NO: 65). The slight shift in position of the protein DNA complex is likely due to the deletion of the C-terminus and charge removal.

FIG. 2 shows diagrams of the systems used in Example 3a and 3b to investigate pore blocking by a strand of DNA covalently attached to the nanopore. In Example 3a (left-hand side) a nanopore (labelled X) is covalently attached to a short strand of DNA (labelled A) which contains two uracil's labelled with azidohexanoic acid and which has a thiol group at the 5′ end of the strand. A can be covalently attached to a sequence (labelled B), which contains alkyne residues, has a thiol at the 5′ end and has a Cy3 fluorescent tag at the 3′ end. This covalent attachment occurs by click chemistry by reaction of the alkyne residues in B with the azidohexanoic acid labelled uracil residues in A. The Cy3 fluorescent tag at the 3′ end of B is indicated by a grey square. An exonuclease I mutant enzyme is added in free solution (labelled C). In Example 3b (right-hand side) a nanopore (labelled X) is covalently attached to a short strand of DNA (labelled A) which also contains two U's labelled with azidohexanoic acid. A can be covalently attached to a sequence (labelled D), which contains alkyne residues, has a thiol at the 5′ end and has a Cy3 fluorescent tag at the 3′ end of the strand. This covalent attachment occurs by click chemistry by reaction of the alkyne residues in D with the azidohexanoic acid labelled uracil residues in A. A PhiE polymerase mutant enzyme (labelled E) is also is covalently attached by reaction with the group at the 5′ end of D. The Cy3 fluorescent group at the 3′ end of D is indicated by a grey square. The exonuclease I mutant enzyme is added in free solution (labelled C, SEQ ID NO: 80).

FIG. 3 shows intramolecular blocking of an alpha-hemolysin mutant nanopore (6 subunits of SEQ ID NO: 77 with the mutation N139Q and one subunit of SEQ ID NO: 77 with the mutations N139Q/L135C/E287C, with 5 aspartates, a Flag-tag and H6 tag to aid purification and a DNA strand (SEQ ID NO: 78) reacted by its 5′ end thiol group to position 287 of this subunit) by a DNA strand ((comprising SEQ ID NO: 79, which has a thiol at the 5′ end and a Cy3 fluorescent tag at the 3′ end of the strand) which is covalently attached, via click chemistry, to the DNA (SEQ ID NO: 78 (which has a thiol group at the 5′ end of the strand) which is attached to the mutant nanopore) in the absence of SSB (see FIG. 2, Example 3a for diagram). Multiple pores were allowed to insert into multiple bilayers on a chip system until at least 10% occupancy was achieved. The potential was then cycled accordingly; 5 seconds+150 mV, 1 second-150 m V and 4 seconds 0 mV. The axis labels for the plot shown in this figure are y-axis=relative DNA block current level and x-axis=time(s). Time periods of 10 mins were recorded for each section; section 1 is the control period (400 mM KCl, 25 mM Tris, 10 uM EDTA, pH 7.5), section 3 is the period after Mg²⁺ buffer flush (400 mM KCl, 25 mM Tris, 10 mM MgCl₂, pH7.5) and section 4 is addition of free exonuclease I mutant enzyme (100 nM, SEQ ID NO: 80) to clear the pore by digestion of the analyte DNA (SEQ ID NO: 79, which has a thiol at the 5′ end and a Cy3 fluorescent tag at the 3′ end of the strand). It can be seen that during the control period the DNA (comprising SEQ ID NO. 79, which has a thiol at the 5′ end and a Cy3 fluorescent tag at the 3′ end of the strand) attached to the pore rapidly brings about a DNA block level. No SSB was added in this experiment and flushing of the system with Mg2+ buffer flush continued to show the DNA rapidly blocking the pore. On addition of the free exonuclease I mutant enzyme (SEQ ID NO: 80) the DNA strand (comprising SEQ ID NO: 79, which has a thiol at the 5′ end and a Cy3 fluorescent tag at the 3′ end of the strand) is digested and so the relative block level is increased, as the open pore level is now observed instead of the DNA blocking level.

FIG. 4 shows the effect on intramolecular blocking of an alpha-hemolysin mutant nanopore (6 subunits of SEQ ID NO: 77 with the mutation N139Q and one subunit of SEQ ID NO: 77 with the mutations N139Q/L135C/E287C, with 5 aspartates, a Flag-tag and H6 tag to aid purification and a DNA strand (SEQ ID NO. 78) reacted by its 5′ end thiol to position 287 of this subunit) by a DNA strand ((comprising SEQ ID NO. 79, which has a thiol at the 5′ end and a Cy3 fluorescent tag at the 3′ end of the strand) which is covalently attached, via click chemistry, to the DNA (SEQ ID NO: 78, which also has a thiol group at the 5′ end of the strand) which is attached to the mutant nanopore) upon the addition of EcoSSB-WT (SEQ ID NO.65) (see FIG. 2, Example 3a for diagram). Multiple pores were allowed to insert into multiple bilayers on a chip system until at least 10% occupancy was achieved. The potential was then cycled accordingly; 5 seconds+150 mV, 1 second-150 mV and 4 seconds 0 mV. The axis labels for the plot shown in this figure are y-axis=relative DNA block current level and x-axis=time(s). Time periods of 10 mins were recorded for each section; section 1 is the control period (400 mM KCl, 25 mM Tris, 10 uM EDTA, pH 7.5), section 2 is the SSB period (10 nM, SEQ ID NO: 65), section 3 is the period after Mg²⁺ buffer flush (400 mM KCl, 25 mM Tris, 10 mM MgCl₂, pH 7.5) and section 4 is addition of free exonuclease I mutant enzyme (100 nM, SEQ ID NO: 80) to clear the pore by digestion of the analyte DNA (comprising SEQ ID NO: 79, which has a thiol at the 5′ end and a Cy3 fluorescent tag at the 3′ end of the strand). It can be seen that during the control period the DNA (comprising SEQ ID NO: 79, which has a thiol at the 5′ end and a Cy3 fluorescent tag at the 3′ end of the strand) attached to the pore rapidly brings about a DNA block level. On addition of EcoSSB-WT (SEQ ID NO: 65) the nanopore blocks to a greater current deflection to that observed for the DNA blocking level. This is due to the interaction of the negatively charged C-terminus of the EcoSSB-WT (SEQ ID NO: 65) with the nanopore instead of the DNA. The interaction between EcoSSB-WT (SEQ ID NO: 65) is quite stable as the buffer flush (section 3) does not remove the bound protein. On addition of the free exonuclease I mutant enzyme (SEQ ID NO: 80) the DNA strand (comprising SEQ ID NO: 79, which has a thiol at the 5′ end and a Cy3 fluorescent tag at the 3′ end of the strand) is digested and so the 20 relative block level is increased, as the open pore level is now observed as the DNA has been removed and the SSB is no longer in close association with the nanopore.

FIG. 5 shows the effect on intramolecular blocking of an alpha-hemolysin mutant nanopore (6 subunits of SEQ ID NO: 77 with the mutation N139Q and one subunit of SEQ ID NO: 77 with the mutations N139Q/L135C/E287C, with 5 aspartates, a Flag-tag and H6 tag to aid purification and a DNA strand (SEQ ID NO 78) reacted by its 5′ end thiol to position 287 of this subunit) by a DNA strand ((comprising SEQ ID NO: 79, which has a thiol at the 5′ end and a Cy3 fluorescent tag at the 3′ end of the strand) which is covalently attached, via click chemistry, to the DNA (SEQ ID NO: 78, which also has a thiol group at the 5′ end of the strand) which is attached to the mutant nanopore) upon the addition of EcoSSB-Q152del (SEQ ID NO: 68) (see FIG. 2, Example 3a for diagram). Multiple pores were allowed to insert into multiple bilayers on a chip system until at least 10% occupancy was achieved. The potential was then cycled accordingly; 5 seconds+150 mV, 1 second-150 mV and 4 seconds 0 mV. The axis labels for the plot shown in this figure are y-axis=relative DNA block current level and x-axis=time(s). Time periods of 10 mins were recorded for each section; section 1 is the control period (400 mM KCl, 25 mM Tris, 10 uM EDTA, pH 7.5), section 2 is the SSB period (10 nM, SEQ ID NO: 68), section 3 is the period after Mg²⁺ buffer flush (400 mM KCl, 25 mM Tris, 10 mM MgCl₂, pH 7.5) and section 4 is addition of free exonuclease I mutant enzyme (100 nM, SEQ ID NO: 80) to clear the pore by digestion of the analyte DNA (comprising SEQ ID NO: 79, which has a thiol at the 5′ end and a Cy3 fluorescent tag at the 3′ end of the strand). It can be seen that during the control period the DNA attached to the pore (comprising SEQ ID NO: 79, which has a thiol at the 5′ end and a Cy3 fluorescent tag at the 3′ end of the strand) rapidly brings about a DNA block level. On addition of EcoSSB-Q152del (SEQ ID NO: 68) the DNA block level is abolished similar to that observed for addition of free exonuclease I mutant enzyme (SEQ ID NO. 80). This is because the protein sequesters the DNA (comprising SEQ ID NO: 79, which has a thiol group at the 5′ end and a Cy3 fluorescent tag at the 3′ end of the strand) such that it cannot interact with the pore and block it. The EcoSSB-Q152del (SEQ ID NO: 68) was not observed to block the pore as the WT-EcosSSB (SEQ ID NO: 65) did. The interaction between EcoSSB-Q152del (SEQ ID NO: 68) is quite stable as the buffer flush (section 3) does not remove the bound protein. On addition of the free exonuclease I mutant enzyme (SEQ ID NO: 80) the DNA strand (comprising SEQ ID NO: 79, which has a thiol at the 5′ end and a Cy3 fluorescent tag at the 3′ end of the strand) is digested, the open pore level is observed as the DNA has been removed.

FIG. 6 shows the effect on intramolecular blocking of an alpha-hemolysin mutant nanopore (6 subunits of SEQ ID NO: 77 with the mutation N139Q and one subunit of SEQ ID NO. 77 with the mutations N139Q/L135C/E287C and with 5 aspartates, a Flag-tag and H6 tag to aid purification and a DNA strand (SEQ ID NO: 78) reacted by its 5′ end thiol to position 287 of this subunit), by a DNA strand ((comprising SEQ ID NO: 81 which has a thiol at the 5′ end and a Cy3 fluorescent tag at the 3′ end of the strand) which is covalently attached, via click chemistry, to the DNA (SEQ ID NO. 78, which also has a thiol group at the 5′ end of the strand) which is attached to the mutant nanopore) and SEQ ID NO. 81 (which has a thiol at the 5′ end and a Cy3 fluorescent tag at the 3′ end of the strand) is also covalently attached by a thiol group at its 5′ end (SEQ ID NO: 81) to the mutant PhiE polymerase enzyme (SEQ ID NO: 82) at position 373), upon the addition of a WT-SSB that naturally lacks an acidic C-terminus (p5 protein from Phi29 virus, SEQ ID NO: 64) (see FIG. 2 Example 3b for diagram). Multiple nanopores were allowed to insert into multiple bilayers on a chip system until at least 10% occupancy was achieved. The potential was then cycled accordingly: 5 seconds+150 mV, 1 second-150 mV and 4 seconds 0 mV. The axis labels for the plot shown in this figure are y-axis=relative DNA block current level and x-axis=time(s). Time periods of 10 mins were recorded for each section; section 1 is the control period (400 mM KCl, 25 mM Tris, 10 uM EDTA, pH 7.5), section 2 is the 100 nM Phi29 p5 SSB (SEQ ID NO: 64) period, section 3 is the 1 μM Phi29 p5 SSB (SEQ ID NO: 64) period, section 4 is the 10 μM phi29 p5 SSB (SEQ ID NO: 64) period, section 5 is the period after EDTA buffer flush (400 mM KCl, 25 mM Tris, 10 UM EDTA, pH 7.5) and section 6 is addition of the free exonuclease I mutant enzyme ((100 nM, SEQ ID NO: 80) in 400 mM KCl, 25 mM Tris, 10 mM MgCl₂, pH7.5) to clear the pore by digestion of the analyte DNA (comprising SEQ ID NO: 81, which has a thiol at the 5′ end and a Cy3 fluorescent tag at the 3′ end of the strand). It can be seen that during the control period the DNA attached to the pore (comprising SEQ ID NO: 81, which has a thiol at the 5′ end and a Cy3 fluorescent tag at the 3′ end of the strand) rapidly brings about a DNA block level. This blocking continues until addition of Phi29 p5 SSB (SEQ ID NO: 64) reaches 10 UM (section 4), three orders of magnitude more than was required for the EcoSSB-Q152del (SEQ ID NO: 68, FIG. 5). Phi29 p5 SSB (SEQ ID NO: 64) has very dynamic binding to the DNA (comprising SEQ ID NO: 81, which has a thiol at the 5′ end and a Cy3 fluorescent tag at the end of the strand) as a buffer flush (section 5) removed the bound protein. On addition of the free exonuclease I mutant enzyme (SEQ ID NO: 80) the DNA strand is digested and so the relative block level is increased, as the open pore level is now observed as the DNA has been removed. This level is similar to that seen when the SSB bound the DNA strand, except that with the SSB the strand is merely physically constrained from entering the pore and not digested.

FIG. 7 shows the DNA substrate design used in Example 4. The DNA substrate is made up of SEQ ID NO: 70 (labelled A) which is the PhiX 5 kB sense strand which has a 50 spacer unit at the 5′ end, SEQ ID NO. 71 (labelled B) which is the PhiX 5 kB anti-sense strand and SEQ ID NO: 72 (labelled C) which has at the 3′ end of the sequence, six iSp18 spacers attached to two thymine residues and a 3′ cholesterol TEG (indicated by the two black circles).

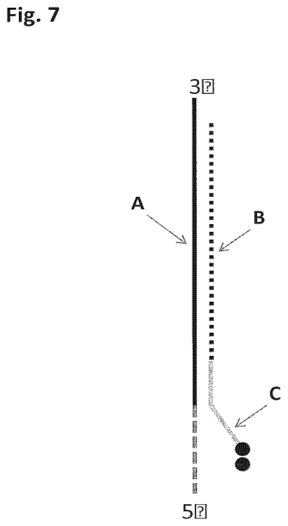

FIG. 8 shows a current trace (y-axis label=current (pA) and x-axis label=time (min)) observed when helicase-controlled 5 KB DNA (SEQ ID NOs 70 (has 50 spacer unit at the 5′ end of the sequence), 71 and 72 (which at the 3′ end of the sequence has six iSp18 spacers attached to two thymine residues and a 3′ cholesterol TEG)) movement was investigated in the presence of EcoSSB-WT (SEQ ID NO: 65). Level 1 corresponds to the open pore level. Level 2 corresponds to the DNA block level. Level 3 corresponds to when EcoSSB-WT (SEQ ID NO: 65) has blocked the nanopore. Addition of EcoSSB-WT (SEQ ID NO: 65) caused the pore to block to a steady level preventing the observation of helicase controlled DNA movement.

FIG. 9 shows a current trace (y-axis label=current (pA) and x-axis label=time (min)) observed when helicase-controlled 5 kB DNA (SEQ ID NOs 70 (has a 50 spacer unit at the 5′ end of the sequence), 71 and 72 (which at the 3′ end of the sequence has six iSp18 spacers attached to two thymine residues and a 3′ cholesterol TEG)) movement was investigated in the presence of EcoSSB-Q152del (SEQ ID NO: 68). Level 1 corresponds to the open pore level. Level 2 corresponds to the DNA block level Addition of EcoSSB-Q152del (SEQ ID NO. 68) facilitated the observation of helicase controlled DNA movement along the entire length of a 5 kB strand of DNA. This data indicates that EcoSSB-Q152del (SEQ ID NO: 68) could be a suitable additive for nanopore DNA sequencing.

FIG. 10 shows a fluorescence assay for testing the DNA binding ability of various transport control proteins, such as a helicase or helicase dimer, and constructs, comprising a transport control protein attached to an SSB. A custom fluorescent substrate was used to assay the ability of various transport control proteins and constructs to bind to single-stranded DNA. The 88 nt single-stranded DNA substrate (1 nM final, SEQ ID NO: 73, labelled A) has a carboxyfluorescein (FAM) base at its 5′ end (circle labelled B). As the transport control protein or construct (labelled C) binds to the oligonucleotide in buffered solution (400 mM NaCl, 10 mM Hepes, pH 8 0, 1 mM MgCl₂), the fluorescence anisotropy (a property relating to the rate of free rotation of the oligonucleotide in solution) increases. The lower the amount of transport control protein or construct needed to affect an increase in anisotropy, the tighter the binding affinity between the DNA and the transport control protein or construct. Situation 1 with no transport control protein or construct bound has a faster rotation and low anisotropy, whereas, situation 2 with the transport control protein or construct bound has slower rotation and high anisotropy. The black bar labelled X corresponds to increasing transport control protein or construct concentration (the thicker the bar the higher the transport control protein or construct concentration).

FIG. 11 shows the change in anisotropy of the DNA oligonucleotide (SEQ ID NO: 73, which has a carboxyfluorescein base at its 5′ end) with increasing amounts of various transport control proteins (y-axis label=Anisotropy (blank subtracted), x-axis label=Protein Concentration (nM)) The data with black square points correspond to the Hel308 Mbu monomer (SEQ ID NO: 10) The data with the empty circles correspond to the Hel308 Mbu A700C 2 kDa dimer (where each monomer unit comprises SEQ ID NO: 10 with the mutation A700C, with one monomer unit being linked to the other via position 700 of each monomer unit using a 2 kDa PEG linker). A lower concentration of the Hel308 Mbu A700C 2 kDa dimer is required to affect an increase in anisotropy, therefore, the dimer has a higher binding affinity for the DNA than the monomer.

FIG. 12 shows the change in anisotropy of the DNA oligonucleotide (SEQ ID NO: 73, which has a carboxyfluorescein base at its 5′ end) with increasing amounts of transport control proteins (y-axis label=Anisotropy (blank subtracted), x-axis label=Protein Concentration (nM)). The data with black square points correspond to the Hel308 Mbu monomer (SEQ ID NO: 10). The data with the empty circles correspond to Hel308 Mbu-GTGSGA-(HhH)2 (where a helicase monomer unit (SEQ ID NO: 10) is attached by the linker sequence GTGSGA to a (HhH)2 domain (SEQ ID NO: 74)) and the data with the empty triangles correspond to Hel308 Mbu-GTGSGA-(HhH)2-(HhH)2 (where a helicase monomer unit (SEQ ID NO: 10) is attached by the linker sequence GTGSGA to a (HhH)2-(HhH)2 domain (SEQ ID NO: 75)). The Hel308 Mbu helicases with additional helix-hairpin-helix binding domains attached show an increase in anisotropy at a lower concentration than the Hel308 Mbu monomer (SEQ ID NO. 10). This indicates that the helicases with additional (HhH)2 binding domains attached (Hel308 Mbu-GTGSGA-(HhH)2 and Hel308 Mbu-GTGSGA-(HhH)2-(HhH)2) have a stronger binding affinity for DNA than Hel308 Mbu monomer. The Hel308 Mbu-GTGSGA-(HhH)2-(HhH)2, which has four HhH domains, was observed to bind DNA more tightly than Hel308 Mbu-GTGSGA-(HhH)2 which only has two HhH domains.

FIG. 13 shows the change in anisotropy of the DNA oligonucleotide (SEQ ID NO: 73, which has a carboxyfluorescein base at its 5′ end) with increasing amounts of various transport control proteins or constructs (y-axis label=Anisotropy (blank subtracted), x-axis label=Protein Concentration (nM)). The data with black square points corresponds to the Hel308 Mbu monomer (SEQ ID NO: 10). The data with the empty circles correspond to Hel308 Mbu-GTGSGA-UL42HV1-1320Del (where a helicase monomer unit (SEQ ID NO: 10) is attached by the linker sequence GTGSGA to UL42HV1-1320Del (SEQ ID NO: 76)), the data with the empty triangles pointing up correspond to Hel308 Mbu-GTGSGA-gp32RB69CD (where a helicase monomer unit (SEQ ID NO: 10) is attached by the linker sequence GTGSGA to gp32RB69CD (SEQ ID NO: 59)) and the data with empty triangles pointing down correspond to Hel308 Mbu-GTGSGA-gp2.5T7-R211Del (where a helicase monomer unit (SEQ ID NO: 10) is attached by the linker sequence GTGSGA to gp2.5T7-R211Del (SEQ ID NO: 60)). All of the constructs (Hel308 Mbu-GTGSGA-UL42HV1-1320Del, Hel308 Mbu-GTGSGA-gp32RB69CD and Hel308 Mbu-GTGSGA-gp2.5T7-R211Del) show an increase in anisotropy at a lower concentration than the monomer Hel308 Mbu. This indicates that the constructs have a stronger binding affinity for DNA than the transport control protein-Hel308 Mbu monomer.

FIG. 14 shows the change in anisotropy of the DNA oligonucleotide (SEQ ID NO: 73, which has a carboxyfluorescein base at its 5′ end) with increasing amounts of a transport control protein or a construct (y-axis label: Anisotropy (blank subtracted), x-axis label=Protein Concentration (nM)). The data with black square points correspond to the Hel308 Mbu monomer (SEQ ID NO: 10). The data with the empty circles correspond to (gp32-RB69CD)-Hel308 Mbu (where the gp32-RB69CD (SEQ ID NO: 59) is attached by the linker sequence GTGSGT to the helicase monomer unit (SEQ ID NO: 10)). The construct (gp32-RB69CD)-Hel308 Mbu shows an increase in anisotropy at a lower concentration than the monomer Hel308 Mbu, indicating tighter binding to the DNA was observed with the construct in comparison to the transport control protein-Hel308 Mbu monomer.

FIG. 15 shows relative equilibrium dissociation constants (K_{d}) (with respect to the Hel308 Mbu monomer) for various transport control proteins and constructs, obtained through fitting two phase dissociation binding curves through the data shown in FIGS. 11-14 using Graphpad Prism software (y-axis label=Relative K_{d}, x-axis label=Ref. Number). The reference numbers correspond to the following Hel308 (Mbu) constructs-3614=Hel308 (Mbu), 3694=(gp32-RB69CD)-Hel308 Mbu, 3733=Hel308 (Mbu)-A700C 2 kDa PEG dimer, 4401=Hel308 (Mbu)-GTGSGA-(HhH)2, 4402=Hel308 (Mbu)-GTGSGA-(HhH)2-(HhH)2, 4394Hel308 (Mbu)-GTGSGA-gp32RB69CD, 4395=Hel308 (Mbu)-GTGSGA-gp2.5T7-R112Del and 4396-Hel308 (Mbu)-GTGSGA-UL42HV1-1320Del. All of the transport control proteins and constructs (Hel308 Mbu A700C 2 kDa dimer, Hel308 Mbu-GTGSGA-(HhH)2, Hel308 Mbu-GTGSGA-(HhH)2-(HhH)2, Hel308 Mbu-GTGSGA-UL42HV1-1320Del, Hel308 Mbu-GTGSGA-gp32RB69CD, Hel308 Mbu-GTGSGA-gp2.5T7-R211Del and (gp32-RB69CD)-Hel308 Mbu) show a lower equilibrium dissociation constant than the transport control protein-Hel308 Mbu monomer.

### DESCRIPTION OF THE SEQUENCE LISTING

SEQ ID NO: 1 shows the codon optimised polynucleotide sequence encoding the MS-B1 mutant MspA monomer. This mutant lacks the signal sequence and includes the following mutations: D90N, D91N, D93N, D118R, D134R and E139K.

SEQ ID NO: 2 shows the amino acid sequence of the mature form of the MS-B1 mutant of the MspA monomer. This mutant lacks the signal sequence and includes the following mutations: D90N, D91N, D93N, D118R, D134R and E139K.

SEQ ID NO: 3 shows the polynucleotide sequence encoding one monomer of α-hemolysin-E111N/K147N (α-HL-NN; Stoddart et al., PNAS, 2009; 106 (19) 7702-7707).

SEQ ID NO: 4 shows the amino acid sequence of one monomer of α-HL-NN.

SEQ ID NOs: 5 to 7 show the amino acid sequences of MspB, C and D.

SEQ ID NO: 8 shows the amino acid sequence of the Hel308 motif.

SEQ ID NO: 9 shows the amino acid sequence of the extended Hel308 motif.

SEQ ID NO. 10 shows the amino acid sequence of Hel308 Mbu.

SEQ ID NO: 11 shows the Hel308 motif of Hel308 Mbu and Hel308 Mhu.

SEQ ID NO: 12 shows the extended Hel308 motif of Hel308 Mbu and Hel308 Mhu.

SEQ ID NO: 13 shows the amino acid sequence of Hel308 Csy.

SEQ ID NO: 14 shows the Hel308 motif of Hel308 Csy.

SEQ ID NO: 15 shows the extended Hel308 motif of Hel308 Csy.

SEQ ID NO: 16 shows the amino acid sequence of Hel308 Tga.

SEQ ID NO: 17 shows the Hel308 motif of Hel308 Tga.

SEQ ID NO: 18 shows the extended Hel308 motif of Hel308 Tga.

SEQ ID NO: 19 shows the amino acid sequence of Hel308 Mhu.

SEQ ID NO: 20 shows the RecD-like motif I.

SEQ ID NOs: 21 to 23 show the extended RecD-like motif I.

SEQ ID NO: 24 shows the RecD motif I.

SEQ ID NO: 25 shows a preferred RecD motif I, namely G-G-P-G-T-G-K-T.

SEQ ID NO:s 26 to 28 show the extended RecD motif I.

SEQ ID NO: 29 shows the RecD-like motif V.

SEQ ID NO: 30 shows the RecD motif V.

SEQ ID NOs: 31 to 38 show the MobF motif III.

SEQ ID NOs: 39 to 45 show the MobQ motif III.

SEQ ID NO: 46 shows the amino acid sequence of TraI Eco.

SEQ ID NO: 47 shows the RecD-like motif I of TraI Eco.

SEQ ID NO: 48 shows the RecD-like motif V of TraI Eco.

SEQ ID NO: 49 shows the MobF motif Ill of TraI Eco.

SEQ ID NO: 50 shows the XPD motif V.

SEQ ID NO. 51 shows XPD motif VI.

SEQ ID NO: 52 shows the amino acid sequence of XPD Mbu.

SEQ ID NO: 53 shows the XPD motif V of XPD Mbu.

SEQ ID NO: 54 shows XPD motif VI of XPD Mbu

SEQ ID NO: 55 shows the amino acid sequence of the ssb from the bacteriophage T4, which is encoded by the gp32 gene

SEQ ID NO: 56 shows the amino acid sequence of the ssb from the bacteriophage RB69, which is encoded by the gp32 gene.

SEQ ID NO: 57 shows the amino acid sequence of the ssb from the bacteriophage T7, which is encoded by the gp2.5 gene.

SEQ ID NO: 58 shows the amino acid sequence of Phi29 DNA polymerase.

SEQ ID NO. 59 shows the amino acid sequence of the ssb from the bacteriophage RB69, i.e. SEQ ID NO: 56, with its C terminus deleted (gp32RB69CD).

SEQ ID NO: 60 shows the amino acid sequence (from 1 to 210) of the ssb from the bacteriophage T7 (gp2.5T7-R211Del). The full length protein is shown in SEQ ID NO: 57.

SEQ ID NO: 61 shows the amino acid sequence of the 5th domain of Hel308 Hla.

SEQ ID NO: 62 shows the amino acid sequence of the 5″ domain of Hel308 Hvo.

SEQ ID NO: 63 shows the amino acid sequence of the human mitochondrial SSB (HsmtSSB).

SEQ ID NO: 64 shows the amino acid sequence of the p5 protein from Phi29 DNA polymerase.

SEQ ID NO: 65 shows the amino acid sequence of the wild-type SSB from E. coli (EcoSSB-WT).

SEQ ID NO: 66 shows the amino acid sequence of EcoSSB-CterAla.

SEQ ID NO. 67 shows the amino acid sequence of EcoSSB-CterNGGN.

SEQ ID NO: 68 shows the amino acid sequence of EcoSSB-Q152del.

SEQ ID NO: 69 shows the amino acid sequence of EcoSSB-G117del SEQ ID NO: 70 shows the polynucleotide sequence, for PhiX 5 kB sense strand, which is used in Example 4.

SEQ ID NO: 71 shows the polynucleotide sequence, for PhiX 5 kB anti-sense strand, which is used in Example 4.

SEQ ID NO: 72 shows the polynucleotide sequence of a short strand of DNA which is used in Example 4.

SEQ ID NO: 73 shows the polynucleotide sequence of a DNA strand used in a transport control protein fluorescent assay.

SEQ ID NO. 74 shows the amino acid sequence of the (HhH)2 domain.

SEQ ID NO: 75 shows the amino acid sequence of the (HhH)2-(HhH)2 domain.

SEQ ID NO: 76 shows the amino acid sequence (from 1 to 319) of the UL42 processivity factor from the Herpes virus 1.

SEQ ID NO: 77 shows the amino acid sequence of one subunit of wild-type (WT) α-hemolysin

SEQ ID NO: 78 shows a polynucleotide sequence that contains two uracils which are labelled with azidohexanoic acid and is used in Examples 3a and 3b.

SEQ ID NO: 79 shows a polynucleotide sequence which is used in Example 3a.

SEQ ID NO: 80 shows the amino acids sequence of a mutant EcoExoI with all of its natural cysteines removed, an additional cysteine mutation included at A83C and two Strep tags for purification.

SEQ ID NO: 81 shows a polynucleotide sequence, that contains two alkyne residues (shown as n in sequence), which is used in Example 3b.

SEQ ID NO: 82 shows the amino acid sequence of a PhiE DNA polymerase mutant (PhiE T373C/C22A/C455A/C530A) with a STrEP tag at the C-terminal end.

SEQ ID NO: 83 shows a polynucleotide sequence used in Example 2 SEQ ID NO: 84 shows the GTGSGA linker.

SEQ ID NO: 85 shows the GTGSGT linker.

SEQ ID NOs: 86 to 95 show the TraI sequences shown in Table 5.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “a SSB” includes “SSBs”, reference to “a helicase” includes two or more such helicases, reference to “a transmembrane pore” includes two or more such pores, and the like.

All publications, patents and patent applications cited herein, whether supra or infra, are hereby incorporated by reference in their entirety.

### METHODS OF THE INVENTION

The invention provides a method of characterising a target polynucleotide. The method comprises contacting the target polynucleotide with a transmembrane pore and a SSB such that the target polynucleotide moves through the pore and the SSB does not move through the pore. The SSB is either an SSB comprising a carboxy-terminal (C-terminal) region which does not have a net negative charge or a modified SSB comprising one or more modifications in its C-terminal region which decreases the net negative charge of the C-terminal region. Such SSBs are described in more detail below. The method then comprises taking one or more measurements as the polynucleotide moves with respect to the pore wherein the measurements are indicative of one or more characteristics of the target polynucleotide and thereby characterising the target polynucleotide. The target polynucleotide is preferably contacted with the pore and the SSB on the same side of the membrane.

The method of the invention is advantageous. Specifically, the ability of the SSB to bind the target polynucleotide without blocking the pore is advantageous for maintaining a high rate of experimental throughput. A target polynucleotide is unlikely to pass through a blocked pore. In an experiment which uses an array of multiple pores, the throughput is reduced by each blocked pore. The pores may be “permanently” blocked, ie. for the duration of the experiment without intervention, but it may be possible to unblock the pores by altering experimental conditions, such as reversing the potential. However, the alteration of conditions increases the length and complexity of the experiment and may not successfully unblock the pores. In a single pore experiment, the permanent blocking of the pore results in a failure to acquire any characterizing data.

The method is preferably carried out with a potential applied across the pore. As discussed in more detail below, the applied potential typically results in the formation of a complex between the pore and the SSB. The applied potential may be a voltage potential. Alternatively, the applied potential may be a chemical potential. An example of this is using a salt gradient across an amphiphilic layer. A salt gradient is disclosed in Holden et al., J Am Chem Soc 2007 Jul. 11; 129 (27): 8650-5

In some instances, the current passing through the pore as the polynucleotide moves with respect to the pore is used to determine the sequence of the target polynucleotide. This is Strand Sequencing.

### Target Polynucleotide

The method of the invention is for characterising a target polynucleotide. A polynucleotide, such as a nucleic acid, is a macromolecule comprising two or more nucleotides. The polynucleotide or nucleic acid may comprise any combination of any nucleotides. The nucleotides can be naturally occurring or artificial. One or more nucleotides in the target polynucleotide can be oxidized or methylated One or more nucleotides in the target polynucleotide may be damaged. For instance, the polynucleotide may comprise a pyrimidine dimer. Such dimers are typically associated with damage by ultraviolet light and are the primary cause of skin melanomas. One or more nucleotides in the target polynucleotide may be modified, for instance with a label or a tag. Suitable labels are described above. The target polynucleotide may comprise one or more spacers.

A nucleotide typically contains a nucleobase, a sugar and at least one phosphate group. The nucleobase is typically heterocyclic. Nucleobases include, but are not limited to, purines and pyrimidines and more specifically adenine, guanine, thymine, uracil and cytosine. The sugar is typically a pentose sugar. Nucleotide sugars include, but are not limited to, ribose and deoxyribose. The nucleotide is typically a ribonucleotide or deoxyribonucleotide. The nucleotide typically contains a monophosphate, diphosphate or triphosphate. Phosphates may be attached on the 5′ or 3′ side of a nucleotide.

Nucleotides include, but are not limited to, adenosine monophosphate (AMP), guanosine monophosphate (GMP), thymidine monophosphate (TMP), uridine monophosphate (UMP), cytidine monophosphate (CMP), cyclic adenosine monophosphate (cAMP), cyclic guanosine monophosphate (cGMP), deoxyadenosine monophosphate (dAMP), deoxyguanosine monophosphate (dGMP), deoxythymidine monophosphate (dTMP), deoxyuridine monophosphate (dUMP) and deoxycytidine monophosphate (dCMP). The nucleotides are preferably selected from AMP, TMP, GMP, CMP, UMP, dAMP, dTMP, dGMP, dCMP and dUMP.

A nucleotide may be abasic (i.e. lack a nucleobase). A nucleotide may also lack a nucleobase and a sugar (i.e. is a C3 spacer).

The nucleotides in the polynucleotide may be attached to each other in any manner. The nucleotides are typically attached by their sugar and phosphate groups as in nucleic acids. The nucleotides may be connected via their nucleobases as in pyrimidine dimers.

The polynucleotide may be single stranded or double stranded. At least a portion of the polynucleotide is preferably single stranded.

The polynucleotide can be a nucleic acid, such as deoxyribonucleic acid (DNA) or ribonucleic acid (RNA). The target polynucleotide can comprise one strand of RNA hybridized to one strand of DNA. The polynucleotide may be any synthetic nucleic acid known in the art, such as peptide nucleic acid (PNA), glycerol nucleic acid (GNA), threose nucleic acid (TNA), locked nucleic acid (LNA) or other synthetic polymers with nucleotide side chains.

The whole or only part of the target polynucleotide may be characterised using this method. The target polynucleotide can be any length. For example, the polynucleotide can be at least 10, at least 50, at least 100, at least 150, at least 200, at least 250, at least 300, at least 400 or at least 500 nucleotide pairs in length. The polynucleotide can be 1000 or more nucleotide pairs, 5000 or more nucleotide pairs in length or 100000 or more nucleotide pairs in length.

The target polynucleotide is present in any suitable sample. The invention is typically carried out on a sample that is known to contain or suspected to contain the target polynucleotide. Alternatively, the invention may be carried out on a sample to confirm the identity of one or more target polynucleotides whose presence in the sample is known or expected.

The sample may be a biological sample. The invention may be carried out in vitro on a sample obtained from or extracted from any organism or microorganism. The organism or microorganism is typically archaeal, prokaryotic or eukaryotic and typically belongs to one of the five kingdoms: plantae, animalia, fungi, monera and protista. The invention may be carried out in vitro on a sample obtained from or extracted from any virus. The sample is preferably a fluid sample. The sample typically comprises a body fluid of the patient. The sample may be urine, lymph, saliva, mucus or amniotic fluid but is preferably blood, plasma or serum. Typically, the sample is human in origin, but alternatively it may be from another mammal animal such as from commercially farmed animals such as horses, cattle, sheep or pigs or may alternatively be pets such as cats or dogs. Alternatively a sample of plant origin is typically obtained from a commercial crop, such as a cereal, legume, fruit or vegetable, for example wheat, barley, oats, canola, maize, soya, rice, bananas, apples, tomatoes, potatoes, grapes, tobacco, beans, lentils, sugar cane, cocoa, cotton.

The sample may be a non-biological sample. The non-biological sample is preferably a fluid sample. Examples of a non-biological sample include surgical fluids, water such as drinking water, sea water or river water, and reagents for laboratory tests.

The sample is typically processed prior to being assayed, for example by centrifugation or by passage through a membrane that filters out unwanted molecules or cells, such as red blood cells. The sample may be measured immediately upon being taken. The sample may also be typically stored prior to assay, preferably below −70° C.

### Transmembrane Pore

A transmembrane pore is a structure that crosses the membrane to some degree. It permits hydrated ions driven by an applied potential to flow across or within the membrane. The transmembrane pore typically crosses the entire membrane so that hydrated ions may flow from one side of the membrane to the other side of the membrane. However, the transmembrane pore does not have to cross the membrane. It may be closed at one end. For instance, the pore may be a well in the membrane along which or into which hydrated ions may flow.

The pore may be biological or artificial. Suitable pores include, but are not limited to, protein pores, polynucleotide pores and solid state pores.

The pore allows the target polynucleotide, but not the SSB to move through it. The barrel or channel of the pore preferably has a diameter of less than 10 nm, such as less than 7 nm or less than 5 nm, at its narrowest point.

Any membrane may be used in accordance with the invention. Suitable membranes are well-known in the art. The membrane is preferably an amphiphilic layer. An amphiphilic layer is a layer formed from amphiphilic molecules, such as phospholipids, which have both at least one hydrophilic portion and at least one lipophilic or hydrophobic portion. The amphiphilic layer may be a monolayer or a bilayer. The amphiphilic molecules may be synthetic or naturally occurring. Non-naturally occurring amphiphiles and amphiphiles which form a monolayer are known in the art and include, for example, block copolymers (Gonzalez-Perez et al., Langmuir, 2009, 25, 10447-10450). Block copolymers are polymeric materials in which two or more monomer sub-units that are polymerized together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e. lipophilic), whilst the other sub-unit(s) are hydrophilic whilst in aqueous media. In this case, the block copolymer may possess amphiphilic properties and may form a structure that mimics a biological membrane. The block copolymer may be a diblock (consisting of two monomer sub-units), but may also be constructed from more than two monomer sub-units to form more complex arrangements that behave as amphiphiles. The copolymer may be a triblock, tetrablock or pentablock copolymer.

The amphiphilic layer is typically a planar lipid bilayer or a supported bilayer.

The amphiphilic layer is typically a lipid bilayer Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer or a liposome. The lipid bilayer is preferably a planar lipid bilayer. Suitable lipid bilayers are disclosed in International Application No. PCT/GB08/000563 (published as WO 2008/102121), International Application No. PCT/GB08/004127 (published as WO 2009/077734) and International Application No. PCT/GB2006/001057 (published as WO 2006/100484).

Methods for forming lipid bilayers are known in the art. Suitable methods are disclosed in the Example. Lipid bilayers are commonly formed by the method of Montal and Mueller (Proc. Natl Acad Sci. USA., 1972; 69: 3561-3566), in which a lipid monolayer is carried on aqueous solution/air interface past either side of an aperture which is perpendicular to that interface.

The method of Montal & Mueller is popular because it is a cost-effective and relatively straightforward method of forming good quality lipid bilayers that are suitable for protein pore insertion. Other common methods of bilayer formation include tip-dipping, painting bilayers and patch-clamping of liposome bilayers.

In a preferred embodiment, the lipid bilayer is formed as described in International Application No. PCT/GB08/004127 (published as WO 2009/077734).

In another preferred embodiment, the membrane is a solid state layer. A solid-state layer is not of biological origin. In other words, a solid state layer is not derived from or isolated from a biological environment such as an organism or cell, or a synthetically manufactured version of a biologically available structure. Solid state layers can be formed from both organic and inorganic materials including, but not limited to, microelectronic materials, insulating materials such as Si₃N₄, Al₂O₃, and SiO, organic and inorganic polymers such as polyamide, plastics such as Teflon® or elastomers such as two-component addition-cure silicone rubber, and glasses. The solid state layer may be formed from monatomic layers, such as graphene, or layers that are only a few atoms thick. Suitable graphene layers are disclosed in International Application No. PCT/US2008/010637 (published as WO 2009/035647).

The method is typically carried out using (i) an artificial amphiphilic layer comprising a pore, (ii) an isolated, naturally-occurring lipid bilayer comprising a pore, or (iii) a cell having a pore inserted therein. The method is typically carried out using an artificial amphiphilic layer, such as an artificial lipid bilayer. The layer may comprise other transmembrane and/or intramembrane proteins as well as other molecules in addition to the pore. Suitable apparatus and conditions are discussed below. The method of the invention is typically carried out in vitro.

The polynucleotide may be coupled to the membrane. This may be done using any known method. If the membrane is an amphiphilic layer, such as a lipid bilayer (as discussed in detail above), the polynucleotide is preferably coupled to the membrane via a polypeptide present in the membrane or a hydrophobic anchor present in the membrane. The hydrophobic anchor is preferably a lipid, fatty acid, sterol, carbon nanotube or amino acid.

The polynucleotide may be coupled directly to the membrane. The polynucleotide is preferably coupled to the membrane via a linker. Preferred linkers include, but are not limited to, polymers, such as polynucleotides, polyethylene glycols (PEGs) and polypeptides. If a polynucleotide is coupled directly to the membrane, then some data will be lost as the characterising run cannot continue to the end of the polynucleotide due to the distance between the membrane and the helicase. If a linker is used, then the polynucleotide can be processed to completion. If a linker is used, the linker may be attached to the polynucleotide at any position. The linker is preferably attached to the polynucleotide at the tail polymer.

The coupling may be stable or transient. For certain applications, the transient nature of the coupling is preferred. If a stable coupling molecule were attached directly to either the 5′ or 3′ end of a polynucleotide, then some data will be lost as the characterising run cannot continue to the end of the polynucleotide due to the distance between the bilayer and the helicase's active site. If the coupling is transient, then when the coupled end randomly becomes free of the bilayer, then the polynucleotide can be processed to completion. Chemical groups that form stable or transient links with the membrane are discussed in more detail below. The polynucleotide may be transiently coupled to an amphiphilic layer, such as a lipid bilayer using cholesterol or a fatty acyl chain. Any fatty acyl chain having a length of from 6 to 30 carbon atoms, such as hexadecanoic acid, may be used.

In preferred embodiments, the polynucleotide is coupled to an amphiphilic layer. Coupling of polynucleotides to synthetic lipid bilayers has been carried out previously with various different tethering strategies. These are summarised in Table I below.

TABLE 1

| Attachment group | Type of coupling | Reference |
|---|---|---|
| Thiol | Stable | Yoshina-Ishii, C. and S. G. Boxer (2003). “Arrays of |
| | | mobile tethered vesicles on supported lipid bilayers.” |
| | | J Am Chem Soc 125(13): 3696-7. |
| Biotin | Stable | Nikolov, V., R. Lipowsky, et al. (2007). “Behavior of |
| | | giant vesicles with anchored DNA molecules.” |
| | | Biophys J 92(12): 4356-68 |
| Cholestrol | Transient | Pfeiffer, I. and F. Hook (2004). “Bivalent cholesterol- |
| | | based coupling of oligonucletides to lipid membrane |
| | | assemblies.” J Am Chem Soc 126(33): 10224-5 |
| Lipid | Stable | van Lengerich, B., R. J. Rawle, et al. “Covalent |
| | | attachment of lipid vesicles to a fluid-supported |
| | | bilayer allows observation of DNA-mediated vesicle |
| | | interactions.” Langmuir 26(11): 8666-72 |

Polynucleotides may be functionalized using a modified phosphoramidite in the synthesis reaction, which is easily compatible for the addition of reactive groups, such as thiol, cholesterol, lipid and biotin groups. These different attachment chemistries give a suite of attachment options for polynucleotides. Each different modification group tethers the polynucleotide in a slightly different way and coupling is not always permanent so giving different dwell times for the polynucleotide to the bilayer. The advantages of transient coupling are discussed above.

Coupling of polynucleotides can also be achieved by a number of other means provided that a reactive group can be added to the polynucleotide. The addition of reactive groups to either end of DNA has been reported previously. A thiol group can be added to the 5′ of ssDNA using polynucleotide kinase and ATPγS (Grant, G. P. and P. Z. Qin (2007). “A facile method for attaching nitroxide spin labels at the 5′ terminus of nucleic acids.” Nucleic Acids Res 35 (10): e77). A more diverse selection of chemical groups, such as biotin, thiols and fluorophores, can be added using terminal transferase to incorporate modified oligonucleotides to the 3′ of ssDNA (Kumar, A., P. Tehen, et al. (1988). “Nonradioactive labeling of synthetic oligonucleotide probes with terminal deoxynucleotidyl transferase.” Anal Biochem 169 (2): 376-82).

Alternatively, the reactive group could be considered to be the addition of a short piece of DNA complementary to one already coupled to the bilayer, so that attachment can be achieved via hybridisation. Ligation of short pieces of ssDNA have been reported using T4 RNA ligase I (Troutt, A. B., M. G. McHeyzer-Williams, et al. (1992). “Ligation-anchored PCR: a simple amplification technique with single-sided specificity” Proc Natl Acad Sci USA 89 (20): 9823-5). Alternatively either ssDNA or dsDNA could be ligated to native dsDNA and then the two strands separated by thermal or chemical denaturation. To native dsDNA, it is possible to add either a piece of ssDNA to one or both of the ends of the duplex, or dsDNA to one or both ends. Then, when the duplex is melted, each single strand will have either a 5′ or 3′ modification if ssDNA was used for ligation or a modification at the 5′ end, the 3′ end or both if dsDNA was used for ligation. If the polynucleotide is a synthetic strand, the coupling chemistry can be incorporated during the chemical synthesis of the polynucleotide. For instance, the polynucleotide can be synthesized using a primer with a reactive group attached to it.

A common technique for the amplification of sections of genomic DNA is using polymerase chain reaction (PCR). Here, using two synthetic oligonucleotide primers, a number of copies of the same section of DNA can be generated, where for each copy the 5′ of each strand in the duplex will be a synthetic polynucleotide. By using an antisense primer that has a reactive group, such as a cholesterol, thiol, biotin or lipid, each copy of the amplified target DNA will contain a reactive group for coupling.

The transmembrane pore is preferably a transmembrane protein pore. A transmembrane protein pore is a polypeptide or a collection of polypeptides that permits hydrated ions, such as analyte, to flow from one side of a membrane to the other side of the membrane. In the present invention, the transmembrane protein pore is capable of forming a pore that permits hydrated ions driven by an applied potential to flow from one side of the membrane to the other. The transmembrane protein pore preferably permits analyte such as nucleotides to flow from one side of the membrane, such as a lipid bilayer, to the other. The transmembrane protein pore allows a polynucleotide, such as DNA or RNA, to be moved through the pore.

The transmembrane protein pore may be a monomer or an oligomer. The pore is preferably made up of several repeating subunits, such as 6, 7, 8 or 9 subunits. The pore is preferably a hexameric, heptameric, octameric or nonameric pore.

The transmembrane protein pore typically comprises a barrel or channel through which the ions may flow. The subunits of the pore typically surround a central axis and contribute strands to a transmembrane β barrel or channel or a transmembrane α-helix bundle or channel.

The barrel or channel of the transmembrane protein pore typically comprises amino acids that facilitate interaction with analyte, such as nucleotides, polynucleotides or nucleic acids. These amino acids are preferably located near a constriction of the barrel or channel. The transmembrane protein pore typically comprises one or more positively charged amino acids, such as arginine, lysine or histidine, or aromatic amino acids, such as tyrosine or tryptophan. These amino acids typically facilitate the interaction between the pore and nucleotides, polynucleotides or nucleic acids.

Transmembrane protein pores for use in accordance with the invention can be derived from β-barrel pores or α-helix bundle pores. β-barrel pores comprise a barrel or channel that is formed from β-strands. Suitable β-barrel pores include, but are not limited to, β-toxins, such as α-hemolysin, anthrax toxin and leukocidins, and outer membrane proteins/porins of bacteria, such as Mycobacterium smegmatis porin (Msp), for example MspA MspB, MspC or MspD, outer membrane porin F (OmpF), outer membrane porin G (OmpG), outer membrane phospholipase A and Neisseria autotransporter lipoprotein (NalP). α-helix bundle pores comprise a barrel or channel that is formed from α-helices. Suitable α-helix bundle pores include, but are not limited to, inner membrane proteins and α outer membrane proteins, such as WZA and ClyA toxin. The transmembrane pore may be derived from Msp or from α-hemolysin (α-HL).

The transmembrane protein pore is preferably derived from Msp, preferably from MspA. Such a pore will be oligomeric and typically comprises 7, 8, 9 or 10 monomers derived from Msp. The pore may be a homo-oligomeric pore derived from Msp comprising identical monomers. Alternatively, the pore may be a hetero-oligomeric pore derived from Msp comprising at least one monomer that differs from the others. Preferably the pore is derived from MspA or a homolog or paralog thereof.

A monomer derived from Msp typically comprises the sequence shown in SEQ ID NO: 2 or a variant thereof. SEQ ID NO: 2 is the MS-(B1)8 mutant of the MspA monomer. It includes the following mutations: D90N, D91N, D93N, D118R, D134R and E139K. A variant of SEQ ID NO: 2 is a polypeptide that has an amino acid sequence which varies from that of SEQ ID NO: 2 and which retains its ability to form a pore. The ability of a variant to form a pore can be assayed using any method known in the art. For instance, the variant may be inserted into an amphiphilic layer along with other appropriate subunits and its ability to oligomerise to form a pore may be determined. Methods are known in the art for inserting subunits into membranes, such as amphiphilic layers. For example, subunits may be suspended in a purified form in a solution containing a lipid bilayer such that it diffuses to the lipid bilayer and is inserted by binding to the lipid bilayer and assembling into a functional state. Alternatively, subunits may be directly inserted into the membrane using the “pick and place” method described in M. A. Holden, H. Bayley. J. Am. Chem. Soc. 2005, 127, 6502-6503 and International Application No. PCT/GB2006/001057 (published as WO 2006/100484).

Over the entire length of the amino acid sequence of SEQ ID NO: 2, a variant will preferably be at least 50% homologous to that sequence based on amino acid identity. More preferably, the variant may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 2 over the entire sequence. There may be at least 80%, for example at least 85%, 90% or 95%, amino acid identity over a stretch of 100 or more, for example 125, 150, 175 or 200 or more, contiguous amino acids (“hard homology”).

Standard methods in the art may be used to determine homology. For example the UWGCG Package provides the BESTFIT program which can be used to calculate homology, for example used on its default settings (Devereux et al (1984) Nucleic Acids Research 12, p 387-395). The PILEUP and BLAST algorithms can be used to calculate homology or line up sequences (such as identifying equivalent residues or corresponding sequences (typically on their default settings)), for example as described in Altschul S. F. (1993) J Mol Evol 36:290-300; Altschul, S. F et al (1990) J Mol Biol 215:403-10. Software for performing BLAST analyses is publicly available through the National Center for Biotechnology Information (http://www.ncbi.nlm.nih.gov/).

SEQ ID NO: 2 is the MS-(B1) 8 mutant of the MspA monomer. The variant may comprise any of the mutations in the MspB, C or D monomers compared with MspA. The mature forms of MspB, C and D are shown in SEQ ID NOs. 5 to 7. In particular, the variant may comprise the following substitution present in MspB: A138P. The variant may comprise one or more of the following substitutions present in MspC. A96G, N102E and A138P. The variant may comprise one or more of the following mutations present in MspD: Deletion of G1, L2V, ESQ, L8V, D13G, W21A, D22E, K47T, I49H, I68V, D91G, A96Q, N102D, S103T, V104I, S136K and G141A. The variant may comprise combinations of one or more of the mutations and substitutions from Msp B, C and D. The variant preferably comprises the mutation L88N. A variant of SEQ ID NO: 2 has the mutation L88N in addition to all the mutations of MS-B1 and is called MS-(B2) 8. The pore used in the invention is preferably MS-(B2) 8. A variant of SEQ ID NO: 2 has the mutations G75S/G77S/L88N/Q126R in addition to all the mutations of MS-B1 and is called MS-B2C. The pore used in the invention is preferably MS-(B2) 8 or MS-(B2C) 8.

Amino acid substitutions may be made to the amino acid sequence of SEQ ID NO: 2 in addition to those discussed above, for example up to 1, 2, 3, 4, 5, 10, 20 or 30 substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 2 below. Where amino acids have similar polarity, this can also be determined by reference to the hydropathy scale for amino acid side chains in Table 3.

TABLE 2
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, | Thr | polar, hydrophilic, neutral |
| | charged (+) | Val | aliphatic, hydrophobic, neutral |
| Ile | aliphatic, hydrophobic, neutral | Trp | aromatic, hydrophobic, neutral |
| Lys | polar, hydrophilic, charged(+) | Tyr | aromatic, polar, hydrophobic |
| Leu | aliphatic, hydrophobic, neutral | | |

TABLE 3
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

One or more amino acid residues of the amino acid sequence of SEQ ID NO 2 may additionally be deleted from the polypeptides described above. Up to 1, 2, 3, 4, 5, 10, 20 or 30 residues may be deleted, or more.

Variants may include fragments of SEQ ID NO: 2. Such fragments retain pore forming activity. Fragments may be at least 50, 100, 150 or 200 amino acids in length. Such fragments may be used to produce the pores. A fragment preferably comprises the pore forming domain of SEQ ID NO: 2. Fragments must include one of residues 88, 90, 91, 105, 118 and 134 of SEQ ID NO. 2. Typically, fragments include all of residues 88, 90, 91, 105, 118 and 134 of SEQ ID NO: 2.

One or more amino acids may be alternatively or additionally added to the polypeptides described above. An extension may be provided at the amino terminal or carboxy terminal of the amino acid sequence of SEQ ID NO: 2 or polypeptide variant or fragment thereof. The extension may be quite short, for example from 1 to 10 amino acids in length. Alternatively, the extension may be longer, for example up to 50 or 100 amino acids. A carrier protein may be fused to an amino acid sequence according to the invention. Other fusion proteins are discussed in more detail below.

As discussed above, a variant is a polypeptide that has an amino acid sequence which varies from that of SEQ ID NO: 2 and which retains its ability to form a pore. A variant typically contains the regions of SEQ ID NO: 2 that are responsible for pore formation. The pore forming ability of Msp, which contains a β-barrel, is provided by β-sheets in each subunit. A variant of SEQ ID NO: 2 typically comprises the regions in SEQ ID NO: 2 that form β-sheets. One or more modifications can be made to the regions of SEQ ID NO: 2 that form β-sheets as long as the resulting variant retains its ability to form a pore. A variant of SEQ ID NO: 2 preferably includes one or more modifications, such as substitutions, additions or deletions, within its α-helices and/or loop regions.

The monomers derived from Msp may be modified to assist their identification or purification, for example by the addition of histidine residues (a hist tag), aspartic acid residues (an asp tag), a streptavidin tag or a flag tag, or by the addition of a signal sequence to promote their secretion from a cell where the polypeptide does not naturally contain such a sequence. An alternative to introducing a genetic tag is to chemically react a tag onto a native or engineered position on the pore. An example of this would be to react a gel-shift reagent to a cysteine engineered on the outside of the pore. This has been demonstrated as a method for separating hemolysin hetero-oligomers (Chem Biol 1997 July; 4 (7): 497-505).

The monomer derived from Msp may be labelled with a revealing label. The revealing label may be any suitable label which allows the pore to be detected. Suitable labels are described above.

The monomer derived from Msp may also be produced using D-amino acids. For instance, the monomer derived from Msp may comprise a mixture of L-amino acids and D-amino acids. This is conventional in the art for producing such proteins or peptides.

The monomer derived from Msp contains one or more specific modifications to facilitate nucleotide discrimination. The monomer derived from Msp may also contain other non-specific modifications as long as they do not interfere with pore formation. A number of non-specific side chain modifications are known in the art and may be made to the side chains of the monomer derived from Msp. Such modifications include, for example, reductive alkylation of amino acids by reaction with an aldehyde followed by reduction with NaBH₄, amidination with methylacetimidate or acylation with acetic anhydride.

The monomer derived from Msp can be produced using standard methods known in the art. The monomer derived from Msp may be made synthetically or by recombinant means. For example, the pore may be synthesized by in vitro translation and transcription (IVTT). Suitable methods for producing pores are discussed in International Application Nos. PCT/GB09/001690 (published as WO 2010/004273), PCT/GB09/001679 (published as WO 2010/004265) or PCT/GB10/000133 (published as WO 2010/086603). Methods for inserting pores into membranes are discussed.

The transmembrane protein pore is also preferably derived from α-hemolysin (α-HL). The wild type α-HL pore is formed of seven identical monomers or subunits (i.e. it is heptameric). The sequence of one monomer or subunit of α-hemolysin-NN is shown in SEQ ID NO. 4. The transmembrane protein pore preferably comprises seven monomers each comprising the sequence shown in SEQ ID NO: 4 or a variant thereof. Amino acids 1, 7 to 21, 31 to 34, 45 to 51, 63 to 66, 72, 92 to 97, 104 to 111, 124 to 136, 149 to 153, 160 to 164, 173 to 206, 210 to 213, 217, 218, 223 to 228, 236 to 242, 262 to 265, 272 to 274, 287 to 290 and 294 of SEQ ID NO: 4 form loop regions. Residues 113 and 147 of SEQ ID NO. 4 form part of a constriction of the barrel or channel of α-HL.

In such embodiments, a pore comprising seven proteins or monomers each comprising the sequence shown in SEQ ID NO: 4 or a variant thereof are preferably used in the method of the invention. The seven proteins may be the same (homo-heptamer) or different (hetero-heptamer).

A variant of SEQ ID NO: 4 is a protein that has an amino acid sequence which varies from that of SEQ ID NO: 4 and which retains its pore forming ability. The ability of a variant to form a pore can be assayed using any method known in the art. For instance, the variant may be inserted into an amphiphilic layer, such as a lipid bilayer, along with other appropriate subunits and its ability to oligomerise to form a pore may be determined. Methods are known in the art for inserting subunits into amphiphilic layers, such as lipid bilayers. Suitable methods are discussed above.

The variant may include modifications that facilitate covalent attachment to or interaction with the construct. The variant preferably comprises one or more reactive cysteine residues that facilitate attachment to the construct. For instance, the variant may include a cysteine at one or more of positions 8, 9, 17, 18, 19, 44, 45, 50, 51, 237, 239 and 287 and/or on the amino or carboxy terminus of SEQ ID NO: 4. Preferred variants comprise a substitution of the residue at position 8, 9, 17, 237, 239 and 287 of SEQ ID NO 4 with cysteine (A8C, T9C, N17C, K237C, S239C or E287C) The variant is preferably any one of the variants described in International Application No. PCT/GB09/001690 (published as WO 2010/004273), PCT/GB09/001679 (published as WO 2010/004265) or PCT/GB10/000133 (published as WO 2010/086603).

The variant may also include modifications that facilitate any interaction with nucleotides.

The variant may be a naturally occurring variant which is expressed naturally by an organism, for instance by a Staphylococcus bacterium. Alternatively, the variant may be expressed in vitro or recombinantly by a bacterium such as Escherichia coli. Variants also include non-naturally occurring variants produced by recombinant technology. Over the entire length of the amino acid sequence of SEQ ID NO: 4, a variant will preferably be at least 50% homologous to that sequence based on amino acid identity. More preferably, the variant polypeptide may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 4 over the entire sequence. There may be at least 80%, for example at least 85%, 90% or 95%, amino acid identity over a stretch of 200 or more, for example 230, 250, 270 or 280 or more, contiguous amino acids (“hard homology”). Homology can be determined as discussed above.

Amino acid substitutions may be made to the amino acid sequence of SEQ ID NO: 4 in addition to those discussed above, for example up to 1, 2, 3, 4, 5, 10, 20 or 30 substitutions. Conservative substitutions may be made as discussed above.

One or more amino acid residues of the amino acid sequence of SEQ ID NO: 4 may additionally be deleted from the polypeptides described above. Up to 1, 2, 3, 4, 5, 10, 20 or 30 residues may be deleted, or more.

Variants may be fragments of SEQ ID NO: 4. Such fragments retain pore-forming activity. Fragments may be at least 50, 100, 200 or 250 amino acids in length. A fragment preferably comprises the pore-forming domain of SEQ ID NO: 4. Fragments typically include residues 119, 121, 135, 113 and 139 of SEQ ID NO: 4.

One or more amino acids may be alternatively or additionally added to the polypeptides described above. An extension may be provided at the amino terminus or carboxy terminus of the amino acid sequence of SEQ ID NO: 4 or a variant or fragment thereof. The extension may be quite short, for example from 1 to 10 amino acids in length. Alternatively, the extension may be longer, for example up to 50 or 100 amino acids. A carrier protein may be fused to a pore or variant.

As discussed above, a variant of SEQ ID NO: 4 is a subunit that has an amino acid sequence which varies from that of SEQ ID NO. 4 and which retains its ability to form a pore. A variant typically contains the regions of SEQ ID NO: 4 that are responsible for pore formation. The pore forming ability of α-HL, which contains a β-barrel, is provided by β-strands in each subunit. A variant of SEQ ID NO. 4 typically comprises the regions in SEQ ID NO: 4 that form β-strands. The amino acids of SEQ ID NO: 4 that form β-strands are discussed above. One or more modifications can be made to the regions of SEQ ID NO: 4 that form β-strands as long as the resulting variant retains its ability to form a pore. Specific modifications that can be made to the β-strand regions of SEQ ID NO: 4 are discussed above.

A variant of SEQ ID NO: 4 preferably includes one or more modifications, such as substitutions, additions or deletions, within its α-helices and/or loop regions. Amino acids that form α-helices and loops are discussed above.

The variant may be modified to assist its identification or purification as discussed above.

Pores derived from α-HL can be made as discussed above with reference to pores derived from Msp.

In some embodiments, the transmembrane protein pore is chemically modified. The pore can be chemically modified in any way and at any site. The transmembrane protein pore is preferably chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The transmembrane protein pore may be chemically modified by the attachment of any molecule. For instance, the pore may be chemically modified by attachment of a dye or a fluorophore.

Any number of the monomers in the pore may be chemically modified. One or more, such as 2, 3, 4, 5, 6, 7, 8, 9 or 10, of the monomers is preferably chemically modified as discussed above.

The reactivity of cysteine residues may be enhanced by modification of the adjacent residues. For instance, the basic groups of flanking arginine, histidine or lysine residues will change the pKa of the cysteines thiol group to that of the more reactive S group. The reactivity of cysteine residues may be protected by thiol protective groups such as dTNB. These may be reacted with one or more cysteine residues of the pore before a linker is attached.

The molecule (with which the pore is chemically modified) may be attached directly to the pore or attached via a linker as disclosed in International Application Nos. PCT/GB09/001690 (published as WO 2010/004273), PCT/GB09/001679 (published as WO 2010/004265) or PCT/GB10/000133 (published as WO 2010/086603).

The construct may be covalently attached to the pore. The construct is preferably not covalently attached to the pore. The application of a voltage to the pore and construct typically results in the formation of a sensor that is capable of sequencing target polynucleotides. This is discussed in more detail below.

Any of the proteins described herein, i.e. the transmembrane protein pores or constructs, may be modified to assist their identification or purification, for example by the addition of histidine residues (a his tag), aspartic acid residues (an asp tag), a streptavidin tag, a flag tag, a SUMO tag, a GST tag or a MBP tag, or by the addition of a signal sequence to promote their secretion from a cell where the polypeptide does not naturally contain such a sequence. An alternative to introducing a genetic tag is to chemically react a tag onto a native or engineered position on the pore or construct Δn example of this would be to react a gel-shift reagent to a cysteine engineered on the outside of the pore. This has been demonstrated as a method for separating hemolysin hetero-oligomers (Chem Biol. 1997 July; 4 (7): 497-505).

The pore and/or construct may be labelled with a revealing label. The revealing label may be any suitable label which allows the pore to be detected. Suitable labels include, but are not limited to, fluorescent molecules, radioisotopes, e.g. ¹²⁵I, ³⁵S, enzymes, antibodies, antigens, polynucleotides and ligands such as biotin.

Proteins may be made synthetically or by recombinant means. For example, the pore and/or construct may be synthesized by in vitro translation and transcription (IVTT). The amino acid sequence of the pore and/or construct may be modified to include non-naturally occurring amino acids or to increase the stability of the protein. When a protein is produced by synthetic means, such amino acids may be introduced during production. The pore and/or construct may also be altered following either synthetic or recombinant production.

The pore and/or construct may also be produced using D-amino acids. For instance, the pore or construct may comprise a mixture of L-amino acids and D-amino acids. This is conventional in the art for producing such proteins or peptides.

The pore and/or construct may also contain other non-specific modifications as long as they do not interfere with pore formation or construct function. A number of non-specific side chain modifications are known in the art and may be made to the side chains of the protein(s). Such modifications include, for example, reductive alkylation of amino acids by reaction with an aldehyde followed by reduction with NaBH₄, amidination with methylacetimidate or acylation with acetic anhydride.

The pore and construct can be produced using standard methods known in the art. Polynucleotide sequences encoding a pore or construct may be derived and replicated using standard methods in the art. Polynucleotide sequences encoding a pore or construct may be expressed in a bacterial host cell using standard techniques in the art. The pore and/or construct may be produced in a cell by in situ expression of the polypeptide from a recombinant expression vector. The expression vector optionally carries an inducible promoter to control the expression of the polypeptide. These methods are described in Sambrook, J. and Russell, D. (2001). Molecular Cloning: A Laboratory Manual, 3rd Edition. Cold Spring Harbor Laboratory Press, Cold Spring Harbor, NY.

The pore and/or construct may be produced in large scale following purification by any protein liquid chromatography system from protein producing organisms or after recombinant expression. Typical protein liquid chromatography systems include FPLC, AKTA systems, the Bio-Cad system, the Bio-Rad BioLogic system and the Gilson HPLC system.

### SSB

The method of the invention comprises contacting the target polynucleotide with a SSB. SSBs bind single stranded DNA with high affinity in a sequence non-specific manner. They exist in all domains of life in a variety of forms and bind DNA either as monomers or multimers. Using amino acid sequence alignment and logarithms (such as Hidden Markov models) SSBs can be classified according to their sequence homology. The Pfam family, PF00436, includes proteins that all show sequence similarity to known SSBs. This group of SSBs can then be further classified according to the Structural Classification of Proteins (SCOP). SSBs fall into the following lineage: Class: All beta proteins, Fold; OB-fold, Superfamily: Nucleic acid-binding proteins, Family: Single strand DNA-binding domain, SSB. Within this family SSBs can be classified according to subfamilies, with several type species often characterised within each subfamily.

The SSB may be from a eukaryote, such as from humans, mice, rats, fungi, protozoa or plants, from a prokaryote, such as bacteria and archaea, or from a virus.

Eukariotic SSBs are known as replication protein A (RPAs). In most cases, they are hetero-trimers formed of different size units. Some of the larger units (e.g. RPA70 of Saccharomyces cerevisiae) are stable and bind ssDNA in monomeric form.

Bacterial SSBs bind DNA as stable homo-tetramers (e.g. E. coli, Mycobacterium smegmatis and Helicobacter pylori) or homo-dimers (e.g. Deinococcus radiodurans and Thermotoga maritima). The SSBs from archaeal genomes are considered to be related with eukaryotic RPAs. Few of them, such as the SSB encoded by the crenarchaeote Sulfolobus solfataricus, are homo-tetramers. The SSBs from most other species are closer related to the replication proteins from eukaryotes and are referred to as RPAs. In some of these species they have been shown to be monomeric (Methanococcus jannaschii and Methanothermobacter thermoautotrophicum) Still, other species of Archaea, including Archaeoglobus fulgidus and Methanococcoides burtonii, appear to each contain two open reading frames with sequence similarity to RPAs. There is no evidence at protein level and no published data regarding their DNA binding capabilities or oligomeric state. However, the presence of two oligonucleotide/oligosaccharide (OB) folds in each of these genes (three OB folds in the case of one of the M. burtonii ORFs) suggests that they also bind single stranded DNA.

Viral SSBs bind DNA as monomers. This, as well as their relatively small size renders them amenable to genetic fusion to other proteins, for instance via a flexible peptide linker. Alternatively, the SSBs can be expressed separately and attached to other proteins by chemical methods (e.g. cysteines, unnatural amino-acids). This is discussed in more detail below.

The SSB used in the method of the invention is either (i) an SSB comprising a carboxy-terminal (C-terminal) region which does not have a net negative charge or (ii) a modified SSB comprising one or more modifications in its C-terminal region which decreases the net negative charge of the C-terminal region. Such SSBs do not block the transmembrane pore and therefore allow characterization of the target polynucleotide.

Examples of SSBs comprising a C-terminal region which does not have a net negative charge include, but are not limited to, the human mitochondrial SSB (HsmtSSB; SEQ ID NO: 63), the human replication protein A 70 kDa subunit, the human replication protein A 14 kDa subunit, the telomere end binding protein alpha subunit from Oxytricha nova, the core domain of telomere end binding protein beta subunit from Oxytricha nova, the protection of telomeres protein 1 (Pot1) from Schizosaccharomyces pombe, the human Pot1, the OB-fold domains of BRCA2 from mouse or rat, the p5 protein from phi29 (SEQ ID NO: 64) or a variant of any of those proteins. A variant is a protein that has an amino acid sequence which varies from that of the wild-type protein and which retains single stranded polynucleotide binding activity. Polynucleotide binding activity can be determined using methods known in the art. Suitable methods include, but are not limited to, fluorescence anisotropy, tryptophan fluorescence and electrophoretic mobility shift assay (EMSA). For instance, the ability of a variant to bind a single stranded polynucleotide can be determined as described in the Examples.

A variant of SEQ ID NO 63 or 64 typically has at least 50% homology to SEQ ID NO. 63 or 64 based on amino acid identity over its entire sequence (or any of the % homologies discussed above in relation to pores) and retains single stranded polynucleotide binding activity. A variant may differ from SEQ ID NO: 63 or 64 in any of the ways discussed above in relation to pores. In particular, a variant may have one or more conservative substitutions as shown in Tables 2 and 3.

Examples of SSBs which require one or more modifications in their C-terminal region to decrease the net negative charge include, but are not limited to, the SSB of E. coli (EcoSSB-WT; SEQ ID NO: 65), the SSB of Mycobacterium tuberculosis, the SSB of Deinococcus radiodurans, the SSB of Thermus thermophiles, the SSB from Sulfolobus solfataricus, the human replication protein A 32 kDa subunit (RPA32) fragment, the CDCl3 SSB from Saccharomyces cerevisiae, the Primosomal replication protein N (PriB) from E. coli, the PriB from Arabidopsis thaliana, the hypothetical protein At4g28440, the SSB from T4 (gp32; SEQ ID NO: 55), the SSB from RB69 (gp32, SEQ ID NO: 56), the SSB from T7 (gp2.5; SEQ ID NO: 57) or a variant of any of these proteins. Hence, the SSB used in the method of the invention may be derived from any of these proteins.

In addition to the one or more modifications in the C-terminal region, the SSB used in the method may include additional modifications which are outside the C-terminal region or do not decrease the net negative charge of the C-terminal region. In other words, the SSB used in the method of the invention is derived from a variant of a wild-type protein. A variant is a protein that has an amino acid sequence which varies from that of the wild-type protein and which retains single stranded polynucleotide binding activity. Polynucleotide binding activity can be determined as discussed above.

The SSB used in the invention may be derived from a variant of SEQ ID NO: 55, 56, 57 or 65. In other words, a variant of SEQ ID NO: 55, 56, 57 or 65 may be used as the starting point for the SSB used in the invention, but the SSB actually used further includes one or more modifications in its C-terminal region which decreases the net negative charge of the C-terminal region. A variant of SEQ ID NO: 55, 56, 57 or 65 typically has at least 50% homology to SEQ ID NO: 55, 56, 57 or 65 based on amino acid identity over its entire sequence (or any of the % homologies discussed above in relation to pores) and retains single stranded polynucleotide binding activity. A variant may differ from SEQ ID NO: 55, 56, 57 or 65 in any of the ways discussed above in relation to pores. In particular, a variant may have one or more conservative substitutions as shown in Tables 2 and 3.

It is straightforward to identify the C-terminal region of the SSB in accordance with normal protein N to C nomenclature. The C-terminal region of the SSB is preferably about the last third of the SSB at the C-terminal end, such as the last third of the SSB at the C-terminal end. The C-terminal region of the SSB is more preferably about the last quarter, fifth or eighth of the SSB at the C-terminal end, such as the last quarter, fifth or eighth of the SSB at the C-terminal end. The last third, quarter, fifth or eighth of the SSB may be measured in terms of numbers of amino acids or in terms of actual length of the primary structure of the SSB protein. The length of the various amino acids in the N to C direction are known in the art.

The C-terminal region is preferably from about the last 10 to about the last 60 amino acids of the C-terminal end of the SSB. The C-terminal region is more preferably about the last 15, about the last 20, about the last 25, about the last 30, about the last 35, about the last 40, about the last 45, about the last 50 or about the last 55 amino acids of the C-terminal end of the SSB.

The C-terminal region typically comprises a glycine and/or proline rich region. This proline/glycine rich region gives the C-terminal region flexibility and can be used to identify the C-terminal region.

The method of the invention may use a SSB comprising a C-terminal region which does not have a net negative charge. The C-terminal region may have a net positive charge or a net neutral charge. The net charge of the C-terminal region can be measured using methods known in the art. For instance, the isoelectric point may be used to define the net charge of the C-terminal region. The C-terminal region typically lacks negatively charged amino acids, has the same number of negatively charged and positively charged amino acids or has fewer negatively charged amino acids than positively charged amino acids.

The method of the invention may use a modified SSB comprising one or more modifications in its C-terminal region which decreases the net negative charge of the C-terminal region. In such instances, the C-terminal region is the C-terminal region of the SSB before the one or more modification are made to decrease its negative charge. Before the one or more modifications are made, the C-terminal region has a net negative charge. C-terminal regions having a net negative charge can be identified as discussed above. The C-terminal region typically comprises negatively charged amino acids and/or has more negatively charged amino acids than positively charged amino acids.

The net negative charge of the C-terminal region may be decreased by any means known in the art. The net negative charge of the C-terminal region is decreased in a manner that does not interfere with binding of the modified SSB to the target polynucleotide. A decrease in net negative charge may be measured as discussed above.

The net negative charge is decreased by one or more modifications in the C-terminal region. Any number of modifications, such as 2, 3, 4, 5, 10, 15, 20, 30, 40, 50 or more modifications, may be made,

The one or more modifications are preferably one or more deletions of negatively charged amino acids. Removal of one or more negatively charged amino acids reduces the net negative charge of the C-terminal region. A negatively charged amino acid is an amino acid with a net negative charge. Negatively charged amino acids include, but are not limited to, aspartic acid (D) and glutamic acid (E). Methods for deleting amino acids from proteins, such as SSBs, are well known in the art.

The one or more modifications are preferably deletion of the C-terminal region. Removal of a C-terminal region having a net negative charge decreases the net negative charge at the C-terminus of the resulting modified SSB.

The one or more modifications are preferably one or more substitutions of negatively charged amino acids with one or more positively charged, uncharged, non-polar and/or aromatic amino acids. A positively charged amino acid is an amino acid with a net positive charge. The positively charged amino acid(s) can be naturally-occurring or non-naturally-occurring. The positively charged amino acid(s) may be synthetic or modified. For instance, modified amino acids with a net positive charge may be specifically designed for use in the invention. A number of different types of modification to amino acids are well known in the art.

Preferred naturally-occurring positively charged amino acids include, but are not limited to, histidine (H), lysine (K) and arginine (R). Any number and combination of H. K and/or R may be substituted into the C-terminal region of the SSB.

The uncharged amino acids, non-polar amino acids and/or aromatic amino acids can be naturally occurring or non-naturally-occurring. They may be synthetic or modified. Uncharged amino acids have no net charge. Suitable uncharged amino acids include, but are not limited to, cysteine (C), serine(S), threonine (T), methionine (M), asparagines (N) and glutamine (Q). Non-polar amino acids have non-polar side chains. Suitable non-polar amino acids include, but are not limited to, glycine (G), alanine (A), proline (P), isoleucine (I), leucine (L) and valine (V). Aromatic amino acids have an aromatic side chain. Suitable aromatic amino acids include, but are not limited to, histidine (H), phenylalanine (F), tryptophan (W) and tyrosine (Y). Any number and combination of these amino acids may be substituted into the C-terminal region of the SSB.

The one or more negatively charged amino acids are preferably substituted with alanine (A), valine (V), asparagine (N) or glycine (G). Preferred substitutions include, but are not limited to, substitution of D with A, substitution of D with V, substitution of D with N and substitution of D with G.

The one or more modifications are preferably one or more introductions of positively charged amino acids which neutralise one or more negatively charged amino acids. The neutralisation of negative charge from the C-terminal region of the SSB decreases the net negative charge. The one or more positively charged amino acids may be introduced by addition or substitution. Any amino acid may be substituted with a positively charged amino acid. One or more uncharged amino acids, non-polar amino acids and/or aromatic amino acids may be substituted with one or more positively charged amino acids. Any number of positively charged amino acids may be introduced. The number is typically the same as the number of negatively charged amino acids in the C-terminal region.

The one or more positively charged amino acids may be introduced at any position in the C-terminal region as long as they neutralise the negative charge of the one or more negatively charged amino acids. To effectively neutralise the negative charge, there is typically 5 or fewer amino acids between each positively charged amino acid that is introduced and the negatively charged amino acid it is neutralising. There is preferably 4 or fewer, 3 or fewer or 2 or fewer amino acids between each positively charged amino acid that is introduced and the negatively charged amino acid it is neutralising. There is more preferably one amino acid between each positively charged amino acid that is introduced and the negatively charged amino acid it is neutralising. Each positively charged amino acid is most preferably introduced adjacent to the negatively charged amino acid it is neutralising. Methods for introducing or substituting naturally-occurring amino acids are well known in the art. For instance, methionine (M) may be substituted with arginine (R) by replacing the codon for aspartic acid (GAC) with a codon for alanine (GCC) at the relevant position in a polynucleotide encoding the SSB. The polynucleotide can then be expressed as discussed above.

Methods for introducing or substituting non-naturally-occurring amino acids are also well known in the art. For instance, non-naturally-occurring amino acids may be introduced by including synthetic aminoacyl-tRNAs in the IVTT system used to express the SSB.

Alternatively, they may be introduced by expressing the SSB in E. coli that are auxotrophic for specific amino acids in the presence of synthetic (i.e. non-naturally-occurring) analogues of those specific amino acids. They may also be produced by naked ligation if the SSB is produced using partial peptide synthetisis.

The one or more modifications are preferably one or more chemical modifications of one or more negatively charged amino acids which neutralise their negative charge. For instance, the one or more negatively charged amino acids may be reacted with a carbodiimide.

If the modified SSB is oligomeric, the one or more modifications may be made in one or more of the monomer subunits of the SSB. The one or more modifications are preferably made in all monomer subunits of the SSB.

As discussed above, the modified SSB is preferably derived from the sequence shown in SEQ ID NO. 65 or a variant thereof. The C-terminal region of SEQ ID NO. 65 is typically its last 10 amino acids (amino acids 168 to 177), which comprises four negatively amino acids (four aspartic acids Ds). The four aspartic acids are at positions 170, 172, 173 and 174 of SEQ ID NO: 65.

The general structure of SEQ ID NO: 65's C-terminal region is relatively conserved amongst SSBs which have a C-terminal region having a net negative charge, such as those discussed above. In particular, the C-terminal region of various SSBs comprises a flexible glycine and/or proline rich region followed (in the N to C direction) by several negatively charged amino acids. The C-terminal regions of the SSB from T4 (gp32; SEQ ID NO: 55), the SSB from RB69 (gp32; SEQ ID NO: 56) and the SSB from T7 (gp2.5; SEQ ID NO: 57) are discussed in more detail below.

The modified SSB is more preferably derived from the sequence shown in SEQ ID NO: 65 or a variant thereof and comprises the following modification(s):
a) deletion of one or more of, such as 2, 3 or 4 of, amino acids 170, 172, 173 and 174 in SEQ ID NO: 65;
b) deletion of amino acids 168 to 177 of SEQ ID NO: 65 (i.e. deletion of the C-terminal region);
c) substitution of one or more of, such as 2, 3 or 4 of, amino acids 170, 172, 173 and 174 in SEQ ID NO: 65 with a positively charged, uncharged, non-polar or aromatic amino acid; or
d) substitution of one or more of, such as 2, 3 or 4 of, amino acids 168, 169, 171, 175, 176 and 177 in SEQ ID NO: 65 with a positively charged amino acid. Possible combinations of modifications include (a) and (c), (a) and (d) and (c) and (d).

As discussed above, the modified SSB is preferably derived from the sequence shown in SEQ ID NO: 55 or a variant thereof. The C-terminal region of SEQ ID NO: 55 is typically its last 13 amino acids (amino acids 289 to 301), which comprises six negatively charged amino acids (six aspartic acids Ds). The six aspartic acids are at positions 290, 291, 293, 295, 296 and 300 of SEQ ID NO: 55.

The modified SSB is more preferably derived from the sequence shown in SEQ ID NO: 55 or a variant thereof and comprises the following modification(s)
a) deletion of one or more of, such as 2, 3, 4, 5 or 6 of, amino acids 290, 291, 293, 295, 296 and 300 in SEQ ID NO: 55;
b) deletion of amino acids 289 to 301 of SEQ ID NO: 55 (i.e. deletion of the C-terminal region);
c) substitution of one or more of, such as 2, 3, 4, 5 or 6 of, amino acids 290, 291, 293, 295, 296 and 300 in SEQ ID NO: 55 with a positively charged, uncharged, non-polar or aromatic amino acid; or
d) substitution of one or more of, such as 2, 3, 4, 5, 6 or 7 of, amino acids 289, 292, 294, 297, 298, 299 and 301 in SEQ ID NO: 55 with a positively charged amino acid.

As discussed above, the modified SSB is preferably derived from the sequence shown in SEQ ID NO: 56 or a variant thereof. The C-terminal region of SEQ ID NO: 56 is typically its last 12 amino acids (amino acids 288 to 299), which comprises five negatively charged amino acids (five aspartic acids Ds). The five aspartic acids are at positions 288, 289, 291, 293 and 294 of SEQ ID NO: 56.

The modified SSB is more preferably derived from the sequence shown in SEQ ID NO: 56 or a variant thereof and comprises the following modification(s):
a) deletion of one or more of, such as 2, 3, 4 or S of, amino acids 288, 289, 291, 293 and 294 in SEQ ID NO: 56;
b) deletion of amino acids 288 to 299 of SEQ ID NO: 56 (i.e. deletion of the C-terminal region);
c) substitution of one or more of, such as 2, 3, 4, 5, 6 or 7 of, amino acids 290, 292, 295, 296, 297, 298 and 299 in SEQ ID NO: 56 with a positively charged, uncharged, non-polar or aromatic amino acid; or
d) substitution of one or more of, such as 2, 3, 4, 5, 6 or 7 of, amino acids 290, 292, 295, 296, 297, 298 and 299 in SEQ ID NO: 56 with a positively charged amino acid.

As discussed above, the modified SSB is preferably derived from the sequence shown in SEQ ID NO: 57 or a variant thereof. The C-terminal region of SEQ ID NO: 57 is typically its last 21 amino acids (amino acids 212 to 232), which comprises seven negatively charged amino acids (seven aspartic acids Ds). The seven aspartic acids are at positions 212, 217, 219, 220, 227, 229 and 231 of SEQ ID NO: 57.

The modified SSB is more preferably derived from the sequence shown in SEQ ID NO: 57 or a variant thereof and comprises the following modification(s)
a) deletion of one or more of, such as 2, 3, 4, 5, 6 or 7 of, amino acids 212, 217, 219, 220, 227, 229 and 231 in SEQ ID NO: 57;
b) deletion of amino acids 212 to 232 of SEQ ID NO: 57 (i.e. deletion of the C-terminal region);
c) substitution of one or more of, such as 2, 3, 4, 5, 6, 7, 8, 9, 10, 11, 12, 13 or 14 of, amino acids 213, 214, 215, 216, 218, 221, 222, 223, 224, 225, 226, 228, 230 and 232 in SEQ ID NO: 57 with a positively charged, uncharged, non-polar or aromatic amino acid; or d) substitution of one or more of, such as 2, 3, 4, 5, 6 or 7 of, amino acids 212, 217, 219, 220, 227, 229 and 231 in SEQ ID NO: 57 with a positively charged amino acid.

The modified SSB most preferably comprises a sequence selected from those shown in SEQ ID NOs: 59, 60 and 66 to 69.

### Measuring Characteristics

The method of the invention involves measuring one or more characteristics of the target polynucleotide. The method may involve measuring two, three, four or five or more characteristics of the target polynucleotide. The one or more characteristics are preferably selected from (i) the length of the target polynucleotide, (ii) the identity of the target polynucleotide, (iii) the sequence of the target polynucleotide, (iv) the secondary structure of the target polynucleotide and (v) whether or not the target polynucleotide is modified. Any combination of (i) to (v) may be measured in accordance with the invention.

For (i), the length of the polynucleotide may be measured for example by determining the number of interactions between the target polynucleotide and the pore or the duration of interaction between the target polynucleotide and the pore.

For (ii), the identity of the polynucleotide may be measured in a number of ways. The identity of the polynucleotide may be measured in conjunction with measurement of the sequence of the target polynucleotide or without measurement of the sequence of the target polynucleotide. The former is straightforward; the polynucleotide is sequenced and thereby identified. The latter may be done in several ways. For instance, the presence of a particular motif in the polynucleotide may be measured (without measuring the remaining sequence of the polynucleotide). Alternatively, the measurement of a particular electrical and/or optical signal in the method may identify the target polynucleotide as coming from a particular source.

For (iii), the sequence of the polynucleotide can be determined as described previously. Suitable sequencing methods, particularly those using electrical measurements, are described in Stoddart D et al., Proc Natl Acad Sci, 12; 106 (19): 7702-7, Lieberman K R et al, J Am Chem Soc. 2010; 132 (50): 17961-72, and International Application WO 2000/28312.

For (iv), the secondary structure may be measured in a variety of ways. For instance, if the method involves an electrical measurement, the secondary structure may be measured using a change in dwell time or a change in current flowing through the pore. This allows regions of single-stranded and double-stranded polynucleotide to be distinguished.

For (v), the presence or absence of any modification may be measured. The method preferably comprises determining whether or not the target polynucleotide is modified by methylation, by oxidation, by damage, with one or more proteins or with one or more labels, tags or spacers. Specific modifications will result in specific interactions with the pore which can be measured using the methods described below. For instance, methylcyotsine may be distinguished from cytosine on the basis of the current flowing through the pore during its interation with each nucleotide.

A variety of different types of measurements may be made. This includes without limitation: electrical measurements and optical measurements. Possible electrical measurements include: current measurements, impedance measurements, tunnelling measurements (Ivanov A P et al., Nano Lett. 2011 Jan. 12; 11 (1): 279-85), and FET measurements (International Application WO 2005/124888). Optical measurements may be combined with electrical measurements (Soni G V et al., Rev Sci Instrum. 2010 January; 81 (1): 014301). The measurement may be a transmembrane current measurement such as measurement of ionic current flowing through the pore.

Electrical measurements may be made using standard single channel recording equipment as describe in Stoddart D et al., Proc Natl Acad Sci. 12; 106 (19): 7702-7, Lieberman K R et al, J Am Chem Soc. 2010; 132 (50) 17961-72, and International Application WO-2000/28312. Alternatively, electrical measurements may be made using a multi-channel system, for example as described in International Application WO-2009/077734 and International Application WO-2011/067559.

### Transport Control Protein

Step (a) of the method of the invention preferably further comprises contacting the polynucleotide with a transport control protein such that the transport control protein controls the movement of the target polynucleotide through the pore and wherein the transport control protein does not move through the pore. The transport control protein is preferably derived from a polynucleotide binding enzyme. A polynucleotide binding enzyme is a polypeptide that is capable of binding to a polynucleotide and interacting with and modifying at least one property of the polynucleotide. The enzyme may modify the polynucleotide by cleaving it to form individual nucleotides or shorter chains of nucleotides, such as di- or trinucleotides. The enzyme may modify the polynucleotide by orienting it or moving it to a specific position. The transport control protein does not need to display enzymatic activity as long as it is capable of binding the polynucleotide and controlling its movement. For instance, the protein may be derived from an enzyme that has been modified to remove its enzymatic activity or may be used under conditions which prevent it from acting as an enzyme.

The transport control protein is preferably derived from a nucleolytic enzyme. The enzyme is more preferably derived from a member of any of the Enzyme Classification (EC) groups 3.1.11, 3.1.13, 3.1.14, 3.1.15, 3.1.16, 3.1.21, 3.1.22, 3.1.25, 3.1.26, 3.1.27, 3.1.30 and 3.1.31. The enzyme may be any of those disclosed in International Application No. PCT/GB10/000133 (published as WO 2010/086603).

Preferred enzymes are exonucleases, polymerases, helicases and topoisomerases, such as gyrases. Suitable exonucleases include, but are not limited to, exonuclease I from E. coli, exonuclease III enzyme from E. coli, RecJ from T. thermophilus and bacteriophage lambda exonuclease and variants thereof.

The transport control protein may additionally comprise one or more nucleic acid binding domains or motifs, such as a helix-hairpin-helix (HhH) motif. For example the transport control protein may be a helicase coupled to one, two, three, four or more nucleic acid binding domains such as HhH motifs.

The transport control protein may comprise two or more enzymes coupled together, where the enzymes are the same or different. The transport control protein may additionally comprise a protein which is not an SSB but which is capable of binding to nucleic acid, such as a processivity factor.

The polymerase is preferably a member of any of the Moiety Classification (EC) groups 2.7.7.6, 2.7.7.7, 2.7.7.19, 2.7.7.48 and 2.7.7.49. The polymerase is preferably a DNA-dependent DNA polymerase, an RNA-dependent DNA polymerase, a DNA-dependent RNA polymerase or an RNA-dependent RNA polymerase. The transport control protein is preferably derived from Phi29 DNA polymerase (SEQ ID NO: 58). The transport control protein may comprise the sequence shown in SEQ ID NO: 58 or a variant thereof. A variant of SEQ ID NO: 58 is an enzyme that has an amino acid sequence which varies from that of SEQ ID NO: 58 and which retains polynucleotide binding activity. The variant may include modifications that facilitate binding of the polynucleotide and/or facilitate its activity at high salt concentrations and/or room temperature.

Over the entire length of the amino acid sequence of SEQ ID NO: 58, a variant will preferably be at least 50% homologous to that sequence based on amino acid identity. More preferably, the variant polypeptide may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 58 over the entire sequence. There may be at least 80%, for example at least 85%, 90% or 95%, amino acid identity over a stretch of 200 or more, for example 230, 250, 270 or 280 or more, contiguous amino acids (“hard homology”). Homology is determined as described above. The variant may differ from the wild-type sequence in any of the ways discussed below with reference to SEQ ID NOs: 2 and 4.

Any helicase may be used in the invention. Helicases are often known as translocases and the two terms may be used interchangeably. Suitable helicases are well-known in the art (M. E Fairman-Williams et al., Curr Opin Struct Biol., 2010, 20 (3), 313-324, T. M. Lohman et al, Nature Reviews Molecular Cell Biology, 2008, 9, 391-401) The helicase is typically a member of one of superfamilies 1 to 6. The helicase is preferably a member of any of the Moiety Classification (EC) groups 3.6.1.- and 2.7.7.-. The helicase is preferably an ATP-dependent DNA helicase (EC group 3.6.4.12), an ATP-dependent RNA helicase (EC group 3.6.4.13) or an ATP-independent RNA helicase.

The helicase is preferably capable of binding to the target polynucleotide at an internal nucleotide. An internal nucleotide is a nucleotide which is not a terminal nucleotide in the target polynucleotide. For example, it is not a 3′ terminal nucleotide or a 5′ terminal nucleotide. All nucleotides in a circular polynucleotide are internal nucleotides.

Generally, a helicase which is capable of binding at an internal nucleotide is also capable of binding at a terminal nucleotide, but the tendency for some helicases to bind at an internal nucleotide will be greater than others. For a helicase suitable for use in the invention, typically at least 10% of its binding to a polynucleotide will be at an internal nucleotide. Typically, at least 20%, at least 30%, at least 40% or at least 50% of its binding will be at an internal nucleotide. Binding at a terminal nucleotide may involve binding to both a terminal nucleotide and adjacent internal nucleotides at the same time. For the purposes of the invention, this is not binding to the target polynucleotide at an internal nucleotide. In other words, the helicase used in the invention is not only capable of binding to a terminal nucleotide in combination with one or more adjacent internal nucleotides. The helicase must be capable of binding to an internal nucleotide without concurrent binding to a terminal nucleotide.

A helicase which is capable of binding at an internal nucleotide may bind to more than one internal nucleotide. Typically, the helicase binds to at least 2 internal nucleotides, for example at least 3, at least 4, at least 5, at least 10 or at least 15 internal nucleotides. Typically the helicase binds to at least 2 adjacent internal nucleotides, for example at least 3, at least 4, at least 5, at least 10 or at least 15 adjacent internal nucleotides. The at least 2 internal nucleotides may be adjacent or non-adjacent.

The ability of a helicase to bind to a polynucleotide at an internal nucleotide may be determined by carrying out a comparative assay. The ability of a motor to bind to a control polynucleotide A is compared to the ability to bind to the same polynucleotide but with a blocking group attached at the terminal nucleotide (polynucleotide B). The blocking group prevents any binding at the terminal nucleotide of strand B, and thus allows only internal binding of a helicase.

Examples of helicases which are capable of binding at an internal nucleotide include, but are not limited to, Hel308 Tga, Hel308 Mhu and Hel308 Csy Hence, the molecular motor preferably comprises (a) the sequence of Hel308 Tga (i.e. SEQ ID NO: 16) or a variant thereof or (b) the sequence of Hel308 Csy (i e SEQ ID NO: 13) or a variant thereof or (c) the sequence of Hel308 Mhu (i.e. SEQ ID NO: 19) or a variant thereof. Variants of these sequences are discussed in more detail below. Variants preferably comprise one or more substituted cysteine residues and/or one or more substituted Faz residues to facilitate attachment as discussed above.

The helicase is preferably a Hel308 helicase. Any Hel308 helicase may be used in accordance with the invention. Hel308 helicases are also known as ski2-like helicases and the two terms can be used interchangeably. Suitable Hel308 helicases are disclosed in Table 4 of U.S. Patent Application Nos. 61/549,998 and 61/599,244 and International Application No. PCT/GB2012/052579 (published as WO 2013/057495).

The Hel308 helicase typically comprises the amino acid motif Q-X1-X2-G-R-A-G-R (hereinafter called the Hel308 motif; SEQ ID NO: 8) The Hel308 motif is typically part of the helicase motif VI (Tuteja and Tuteja, Eur. J Biochem 271, 1849-1863 (2004)) X1 may be C, Mor L. X1 is preferably C. X2 may be any amino acid residue. X2 is typically a hydrophobic or neutral residue. X2 may be A, F, M, C, V, L, I, S, T, Por R. X2 is preferably A, F, M, C, V, L, I, S, T or P. X2 is more preferably A, M or L. X2 is most preferably A or M.

The Hel308 helicase preferably comprises the motif Q-X1-X2-G-R-A-G-R-P (hereinafter called the extended Hel308 motif; SEQ ID NO: 9) wherein X1 and X2 are as described above.

The most preferred Hel308 motifs and extended Hel308 motifs are shown in the Table 4 below.

TABLE 4
Preferred Hel308 helicases and their motifs

| | | | % | | |
|---|---|---|---|---|---|
| | | | Identity | | |
| | | | to | | |
| SEQ | | | Hel308 | | Extended |
| ID NO: | Helicase | Names | Mbu | Hel308 motif | Hel308 motif |
| 10 | Hel308 Mbu | Methanococcoides | — | QMAGRAGR | QMAGRAGRP |
| | | burtonii | | (SEQ ID NO: 11) | (SEQ ID NO: 12) |
| 13 | Hel308 Csy | Cenarchaeum | 34% | QLCGRAGR | QLCGRAGRP |
| | | symbiosum | | (SEQ ID NO: 14) | (SEQ ID NO: 15) |
| 16 | Hel308 Tga | Thermococcus | 38% | QMMGRAGR | QMMGRAGRP |
| | | gammatolerans | | (SEQ ID NO: 17) | (SEQ ID NO: 18) |
| | | EJ3 | | | |
| 19 | Hel308 Mhu | Methanospirillum | 40% | QMAGRAGR | QMAGRAGRP |
| | | hungatei JF-1 | | (SEQ ID NO: 11) | (SEQ ID NO: 12) |

The most preferred Hel308 motif is shown in SEQ ID NO: 17. The most preferred extended Hel308 motif is shown in SEQ ID NO: 18. Other preferred Hel308 motifs and extended Hel308 motifs are found in Table 5 of U.S. Patent Application Nos. 61/549,998 and 61/599,244 and International Application No. PCT/GB2012/052579 (published as WO 2013/057495).

The Hel308 helicase preferably comprises the sequence of Hel308 Mbu (i.e. SEQ ID NO. 10) or a variant thereof. The Hel308 helicase more preferably comprises (a) the sequence of Hel308 Tga (i.e. SEQ ID NO: 16) or a variant thereof, (b) the sequence of Hel308 Csy (i.e. SEQ ID NO: 13) or a variant thereof or (c) the sequence of Hel308 Mhu (i.e. SEQ ID NO: 19) or a variant thereof. The Hel308 helicase most preferably comprises the sequence shown in SEQ ID NO: 16 or a variant thereof.

A variant of a Hel308 helicase is an enzyme that has an amino acid sequence which varies from that of the wild-type helicase and which retains polynucleotide binding activity. This can be measured as described above. In particular, a variant of SEQ ID NO: 10, 13, 16 or 19 is an enzyme that has an amino acid sequence which varies from that of SEQ ID NO: 10, 13, 16 or 19 and which retains polynucleotide binding activity.

The variant retains helicase activity. This can be measured in various ways. For instance, the ability of the variant to translocate along a polynucleotide can be measured using electrophysiology, a fluorescence assay or ATP hydrolysis.

The variant may include modifications that facilitate handling of the polynucleotide encoding the helicase and/or facilitate its activity at high salt concentrations and/or room temperature. Variants typically differ from the wild-type helicase in regions outside of the Hel308 motif or extended Hel308 motif discussed above. However, variants may include modifications within these motif(s).

Over the entire length of the amino acid sequence of SEQ ID NO: 10, 13, 16 or 19, a variant will preferably be at least 30% homologous to that sequence based on amino acid identity. More preferably, the variant polypeptide may be at least 40%, at least 45%, at least 50%, at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO. 10, 13, 16 or 19 over the entire sequence. There may be at least 70%, for example at least 80%, at least 85%, at least 90% or at least 95%, amino acid identity over a stretch of 150 or more, for example 200, 300, 400, 500, 600, 700, 800, 900 or 1000 or more, contiguous amino acids (“hard homology”). Homology is determined as described above. The variant may differ from the wild-type sequence in any of the ways discussed below with reference to SEQ ID NOs: 2 and 4.

A variant of SEQ ID NO: 10, 13, 16 or 19 preferably comprises the Hel308 motif or extended Hel308 motif of the wild-type sequence as shown in Table 4 above. However, a variant may comprise the Hel308 motif or extended Hel308 motif from a different wild-type sequence. For instance, a variant of SEQ ID NO: 12 may comprise the Hel308 motif or extended Hel308 motif from SEQ ID NO: 13 (i.e. SEQ ID NO: 14 or 15). Variants of SEQ ID NO: 10, 13, 16 or 19 may also include modifications within the Hel308 motif or extended Hel308 motif of the relevant wild-type sequence. Suitable modifications at X1 and X2 are discussed above when defining the two motifs. A variant of SEQ ID NO: 10, 13, 16 or 19 preferably comprises one or more substituted cysteine residues and/or one or more substituted Faz residues to facilitate attachment as discussed above.

A variant of SEQ ID NO: 10 may lack the first 19 amino acids of SEQ ID NO: 10 and/or lack the last 33 amino acids of SEQ ID NO: 10. A variant of SEQ ID NO: 10 preferably comprises a sequence which is at least 70%, at least 75%, at least 80%, at least 85%, at least 90% or more preferably at least 95%, at least 97% or at least 99% homologous based on amino acid identity with amino acids 20 to 211 or 20 to 727 of SEQ ID NO: 10.

SEQ ID NO: 10 (Hel308 Mbu) contains five natural cysteine residues. However, all of these residues are located within or around the DNA binding grove of the enzyme. Once a DNA strand is bound within the enzyme, these natural cysteine residues become less accessible for external modifications. This allows specific cysteine mutants of SEQ ID NO. 10 to be designed and attached to the SSB using cysteine linkage as discussed above. Preferred variants of SEQ ID NO: 10 have one or more of the following substitutions: A29C, Q221C, Q442C, T569C, A577C, A700C and S708C. The introduction of a cysteine residue at one or more of these positions facilitates cysteine linkage as discussed above. Other preferred variants of SEQ ID NO: 10 have one or more of the following substitutions M2Faz, R10Faz, F15Faz, A29Faz, R185Faz, A268Faz, E284Faz, Y387Faz, F400Faz, Y455Faz, E464Faz, E573Faz, A577Faz, E649Faz, A700Faz, Y720Faz, Q442Faz and S708Faz. The introduction of a Faz residue at one or more of these positions facilitates Faz linkage as discussed above.

The helicase is preferably a RecD helicase. Any RecD helicase may be used in accordance with the invention. The structures of RecD helicases are known in the art (FEBS J. 2008 April; 275 (8). 1835-51. Epub 2008 Mar. 9. ATPase activity of RecD is essential for growth of the Antarctic Pseudomonas syringae Lz4W at low temperature. Satapathy A K, Pavankumar T L, Bhattacharjya S, Sankaranarayanan R, Ray MK; EMS Microbiol Rev. 2009 May: 33 (3): 657-87. The diversity of conjugative relaxases and its application in plasmid classification. Garcillán-Barcia M P, Francia M V, de la Cruz F; J Biol Chem 2011 Apr. 8; 286 (14): 12670-82. Epub 2011 Feb. 2. Functional characterization of the multidomain F plasmid TraI relaxase-helicase Cheng Y, McNamara D E, Miley M. J, Nash R P, Redinbo M R).

The RecD helicase typically comprises the amino acid motif X1-X2-X3-G-X4-X5-X6-X7 (hereinafter called the RecD-like motif I; SEQ ID NO: 20), wherein X1 is G, S or A, X2 is any amino acid, X3 is P, A, S or G, X4 is T, A, V, S or C, X5 is G or A, X6 is K or R and X7 is T or S. X1 is preferably G. X2 is preferably G, I, Y or A. X2 is more preferably G. X3 is preferably P or A. X4 is preferably T, A, V or C. X4 is preferably T. V or C. X5 is preferably G. X6 is preferably K. X7 is preferably T or S. The RecD helicase preferably comprises Q-(X8)₁₆₋₁₈-X1-X2-X3-G-X4-X5-X6-X7 (hereinafter called the extended RecD-like motif I; SEQ ID NOs: 21 to 23), wherein X1 to X7 are as defined above and X8 is any amino acid. There are preferably 16 X8 residues (i.e. (X8)₁₆) in the extended RecD-like motif I. Suitable sequences for (X8)₁₆ can be identified in SEQ ID NOs: 14, 17, 20, 23, 26, 29, 32, 35, 38, 41, 44, 47 and 50 of U.S. Patent Application No. 61/581,332 and SEQ ID NOs: 18, 21, 24, 25, 28, 30, 32, 35, 37, 39, 41, 42 and 44 of International Application No PCT/GB2012/053274 (published as WO 2012/098562).

The RecD helicase preferably comprises the amino acid motif G-G-P-G-Xa-G-K-Xb (hereinafter called the RecD motif I; SEQ ID NO: 24) wherein Xa is T, V or C and Xb is T or S. Xa is preferably T. Xb is preferably T. The Rec-D helicase preferably comprises the sequence G-G-P-G-T-G-K-T (SEQ ID NO: 25). The RecD helicase more preferably comprises the amino acid motif Q-(X8)₁₆₋₁₈-G-G-P-G-Xa-G-K-Xb (hereinafter called the extended RecD motif I; SEQ ID NOs: 26 to 28), wherein Xa and Xb are as defined above and X8 is any amino acid. There are preferably 16 X8 residues (i.e. (X8)₁₆) in the extended RecD motif I. Suitable sequences for (X8)₁₆ can be identified in SEQ ID NOs: 14, 17, 20, 23, 26, 29, 32, 35, 38, 41, 44, 47 and 50 of U.S. Patent Application No. 61/581,332 and SEQ ID NOs: 18, 21, 24, 25, 28, 30, 32, 35, 37, 39, 41, 42 and 44 of International Application No PCT/GB2012/053274 (published as WO 2012/098562).

The RecD helicase typically comprises the amino acid motif X1-X2-X3-X4-X5-(X6)₃-Q-X7 (hereinafter called the RecD-like motif V; SEQ ID NO: 29), wherein X1 is Y, W or F, X2 is A, T, S, M, C or V, X3 is any amino acid, X4 is T, N or S, X5 is A, T, G, S, V or I, X6 is any amino acid and X7 is G or S. X1 is preferably Y. X2 is preferably A, M, C or V. X2 is more preferably A. X3 is preferably I, M or L. X3 is more preferably I or L. X4 is preferably T or S. X4 is more preferably T. X5 is preferably A, V or I. X5 is more preferably V or I. X5 is most preferably V. (X6)₃ is preferably H-K-S, H-M-A, H-G-A or H-R-S. (X6)₃ is more preferably H-K-S. X7 is preferably G. The RecD helicase preferably comprises the amino acid motif Xa-Xb-Xc-Xd-Xe-H-K-S-Q-G (hereinafter called the RecD motif V; SEQ ID NO: 30), wherein Xa is Y, W or F, Xb is A, M, Cor V, Xc is I, M or L, Xd is T or S and Xe is V or I. Xa is preferably Y Xb is preferably A. Xd is preferably T Xd is preferably V. Preferred RecD motifs I are shown in Table 5 of U.S. Patent Application No. 61/581,332 and International Application No. PCT/GB2012/053274 (published as WO 2012/098562). Preferred RecD-like motifs I are shown in Table 7 of U.S. Patent Application No. 61/581,332 and International Application No. PCT/GB2012/053274 (published as WO 2012/098562). Preferred RecD-like motifs V are shown in Tables 5 and 7 of U.S. Patent Application No. 61/581,332 and International Application No. PCT/GB2012/053274 (published as WO 2012/098562).

The RecD helicase is preferably one of the helicases shown in Table 4 or 5 of U.S. Patent Application No. 61/581,332 and International Application No. PCT/GB2012/053274 (published as WO 2012/098562) or a variant thereof. Variants are described in U.S. Patent Application No. 61/581,332 and International Application No. PCT/GB2012/053274 (published as WO 2012/098562).

The RecD helicase is preferably a TraI helicase or a TraI subgroup helicase. TraI helicases and TraI subgroup helicases may contain two RecD helicase domains, a relaxase domain and a C-terminal domain. The TraI subgroup helicase is preferably a TrwC helicase. The TraI helicase or TraI subgroup helicase is preferably one of the helicases shown in Table 6 of U.S. Patent Application No. 61/581,332 and International Application No. PCT/GB2012/053274 (published as WO 2012/098562) or a variant thereof. Variants are described in U.S. Patent Application No. 61/581,332 and International Application No. PCT/GB2012/053274 (published as WO 2012/098562).

The TraI helicase or a TraI subgroup helicase typically comprises a RecD-like motif I as defined above (SEQ ID NO: 20) and/or a RecD-like motif V as defined above (SEQ ID NO: 27). The TraI helicase or a TraI subgroup helicase preferably comprises both a RecD-like motif I (SEQ ID NO: 22) and a RecD-like motif V (SEQ ID NO: 29). The TraI helicase or a TraI subgroup helicase typically further comprises one of the following two motifs:
The amino acid motif H-(X1)₂-X2-R-(X3)₅₋₁₂-H-X4-H (hereinafter called the MobF motif III; SEQ ID NOs: 31 to 38), wherein X1 and X2 are any amino acid and X2 and X4 are independently selected from any amino acid except D, E, K and R. (X1)₂ is of course X1a-X1b. X1a and X1b can be the same of different amino acid. X1a is preferably D or E. X1b is preferably T or D. (X1) 2 is preferably DT or ED. (X1) 2 is most preferably DT. The 5 to 12 amino acids in (X3) 5-12 can be the same or different. X2 and X4 are independently selected from G, P, A, V, L, I, M, C, F, Y, W, H, Q, N, S and T. X2 and X4 are preferably not charged. X2 and X4 are preferably not H. X2 is more preferably N, S or A. X2 is most preferably N. X4 is most preferably F or T. (X3) 5-12 is preferably 6 or 10 residues in length. Suitable embodiments of (X3) 5-12 can be derived from SEQ ID NOs: 58, 62, 66 and 70 shown in Table 7 of U.S. Patent Application No. 61/581,332 and SEQ ID NOs: 61, 65, 69, 73, 74, 82, 86, 90, 94, 98, 102, 110, 112, 113, 114, 117, 121, 124, 125, 129, 133, 136, 140, 144, 147, 151, 152, 156, 160, 164 and 168 of International Application No. PCT/GB2012/053274 (published as WO 2012/098562).
The amino acid motif G-X1-X2-X3-X4-X5-X6-X7-H-(X8)₆₋₁₂-H-X9 (hereinafter called the MobQ motif III; SEQ ID NOs: 39 to 45), wherein X1, X2, X3, X5, X6, X7 and X9 are independently selected from any amino acid except D, E, K and R, X4 is D or E and X8 is any amino acid. X1, X2, X3, X5, X6, X7 and X9 are independently selected from G, P, A, V, L, I, M, C, F, Y, W, H, Q, N, S and T. X1, X2, X3, X5, X6, X7 and X9 are preferably not charged. X1, X2, X3, X5, X6, X7 and X9 are preferably not H. The 6 to 12 amino acids in (X8) 6-12 can be the same or different. Preferred MobF motifs III are shown in Table 7 of U.S. Patent Application No. 61/581,332 and International Application No. PCT/GB2012/053274 (published as WO 2012/098562).

The TraI helicase or TraI subgroup helicase is more preferably one of the helicases shown in Table 6 or 7 of U.S. Patent Application No. 61/581,332 and International Application No. PCT/GB2012/053274 (published as WO 2012/098562) or a variant thereof. The TraI helicase most preferably comprises the sequence shown in SEQ ID NO: 46 or a variant thereof. SEQ ID NO: 46 is TraI Eco (NCBI Reference Sequence: NP_061483.1; Genbank AAQ98619.1; SEQ ID NO: 46). TraI Eco comprises the following motifs: RecD-like motif I (GYAGVGKT; SEQ ID NO: 47), RecD-like motif V (YAITAHGAQG; SEQ ID NO: 48) and Mob F motif III (HDTSRDQEPQLHTH, SEQ ID NO: 49).

The TraI helicase or TraI subgroup helicase more preferably comprises the sequence of one of the helicases shown in Table 5 below, i.e. one of SEQ ID NOs: 46, 86, 90 and 94, or a variant thereof.

TABLE 5
More preferred TraI helicase and TraI subgroup helicases

| | | | | | RecD- | RecD- | |
|---|---|---|---|---|---|---|---|
| | | | | | like | like | Mob F |
| | | | | | motif I | motif V | motif III |
| SEQ | | | | % Identity | (SEQ ID | (SEQ ID | (SEQ ID |
| ID NO | Name | Strain | NCBI ref | to TraI Eco | NO:) | NO:) | NO:) |
| 46 | TraI | Escherichia | NCBI | — | GYAGV | YAITA | HDTSR |
| | Eco | coli | Reference | | GKT | HGAQG | DQEPQ |
| | | | Sequence: | | (47) | (48) | LHTH |
| | | | NP_061483.1 | | | | 49) |
| | | | Genbank | | | | |
| | | | AAQ98619.1 | | | | |
| 86 | TrwC | Citro- | NCBI | 15% | GIAGA | YALNV | HDTNR |
| | Cba | microbium | Reference | | GKS | HMAQG | NQEPN |
| | | bathyo- | Sequence: | | (87) | (88) | LHFH |
| | | marinum | ZP_06861556.1 | | | | (89) |
| | | JL354 | | | | | |
| 90 | TrwC | Halothio- | NCBI | 11.5% | GAAGA | YCITIH | HEDAR |
| | Hne | bacillus | Reference | | GKT | RSQG | TVDDI |
| | | neapoli- | Sequence: | | (91) | (92) | ADPQL |
| | | tanus c2 | YP_003262832.1 | | | | HTH |
| | | | | | | | (93) |
| 94 | TrwC | Erythro- | NCBI | 16% | GIAGA | YALNA | HDTNR |
| | Eli | bacter | Reference | | GKS | HMAQG | NQEPN |
| | | litoralis | Sequence: | | (87) | (95) | LHFH |
| | | HTCC2594 | YP_457045.1 | | | | (89) |

A variant of a RecD helicase, TraI helicase or TraI subgroup helicase is an enzyme that has an amino acid sequence which varies from that of the wild-type helicase and which retains polynucleotide binding activity. In particular, a variant of SEQ ID NO: 46 is an enzyme that has an amino acid sequence which varies from that of SEQ ID NO: 46 and which retains polynucleotide binding activity. This can be measured as described above. The variant retains helicase activity. The variant must work in at least one of the two modes discussed below. Preferably, the variant works in both modes. The variant may include modifications that facilitate handling of the polynucleotide encoding the helicase and/or facilitate its activity at high salt concentrations and/or room temperature. Variants typically differ from the wild-type helicase in regions outside of the motifs discussed above. However, variants may include modifications within these motif(s).

Over the entire length of the amino acid sequence of any one of SEQ ID NO: 46, 86, 90 and 94, a variant will preferably be at least 10% homologous to that sequence based on amino acid identity. More preferably, the variant polypeptide may be at least 20%, at least 25%, at least 30%, at least 40%, at least 45%, at least 50%, at least 55%, at least 60%, at least 65%, at least 70%, at least 75%, at least 80%, at least 85%, at least 90% and more preferably at least 95%, 97% or 99% homologous based on amino acid identity to the amino acid sequence of any one of SEQ ID NOs: 46, 86, 90 and 94 over the entire sequence. There may be at least 70%, for example at least 80%, at least 85%, at least 90% or at least 95%, amino acid identity over a stretch of 150 or more, for example 200, 300, 400, 500, 600, 700, 800, 900 or 1000 or more, contiguous amino acids (“hard homology”). Homology is determined as described above. The variant may differ from the wild-type sequence in any of the ways discussed above with reference to SEQ ID NOs 2 and 4.

A variant of any one of SEQ ID NOs: 46, 86, 90 and 94 preferably comprises the RecD-like motif I and/or RecD-like motif V of the wild-type sequence. However, a variant of SEQ ID NO: 46, 86, 90 or 94 may comprise the RecD-like motif I and/or extended RecD-like motif V from a different wild-type sequence. For instance, a variant may comprise any one of the preferred motifs shown in Tables 5 and 7 of U.S. Patent Application No. 61/581,332. Variants of SEQ ID NOs: 46, 86, 90 and 94 may also include modifications within the RecD-like motifs I and V of the wild-type sequence. A variant of SEQ ID NO: 46, 86, 90 or 94 preferably comprises one or more substituted cysteine residues and/or one or more substituted Faz residues to facilitate attachment as discussed above.

The helicase is preferably an XPD helicase. Any XPD helicase may be used in accordance with the invention. XPD helicases are also known as Rad3 helicases and the two terms can be used interchangeably.

The structures of XPD helicases are known in the art (Cell. 2008 May 30; 133 (5): 801-12. Structure of the DNA repair helicase XPD. Liu H, Rudolf J, Johnson K A, McMahon S A, Oke M, Carter L, McRobbie A M, Brown S E, Naismith J H, White M F). The XPD helicase typically comprises the amino acid motif X1-X2-X3-G-X4-X5-X6-E-G (hereinafter called XPD motif V; SEQ ID NO: 50). X1, X2, X5 and X6 are independently selected from any amino acid except D, E, K and R. X1, X2, X5 and X6 are independently selected from G, P, A, V, L, I, M, C, F, Y, W, H, Q, N, S and T. X1, X2, X5 and X6 are preferably not charged. X1, X2, X5 and X6 are preferably not H. X1 is more preferably V, L, I, S or Y. X5 is more preferably V, L, I, N or F. X6 is more preferably S or A. X3 and X4 may be any amino acid residue. X4 is preferably K, R or T.

The XPD helicase typically comprises the amino acid motif Q-Xa-Xb-G-R-Xc-Xd-R-(Xe)₃-Xf-(Xg)₇-D-Xh-R (hereinafter called XPD motif VI; SEQ ID NO: 51). Xa, Xe and Xg may be any amino acid residue. Xb, Xc and Xd are independently selected from any amino acid except D, E, K and R. Xb, Xc and Xd are typically independently selected from G, P, A, V, L, I, M, C, F, Y, W, H, Q, N, S and T. Xb, Xc and Xd are preferably not charged. Xb, Xc and Xd are preferably not H. Xb is more preferably V, A, L, I or M. Xc is more preferably V, A, L, I, M or C. Xd is more preferably I, H, L, F, M or V. Xf may be D or E. (Xg)₇ is X_{g1}, X_{g2}, X_{g3}, X_{g4}, X_{g5}, X_{g6} and X_{g7}. X_{g2} is preferably G, A, S or C. X_{g5} is preferably F, V, L, I, M, A, W or Y. X_{g6} is preferably L, F, Y, M, I or V. X_{g7} is preferably A, C, V, L, I, M or S.

The XPD helicase preferably comprises XPD motifs V and VI. The most preferred XPD motifs V and VI are shown in Table 5 of U.S. Patent Application No. 61/581,340 and International Application No. PCT/GB2012/053273 (published as WO 2012/098561).

The XPD helicase preferably further comprises an iron sulphide (FeS) core between two Walker A and B motifs (motifs I and II). An FeS core typically comprises an iron atom coordinated between the sulphide groups of cysteine residues. The FeS core is typically tetrahedral.

The XPD helicase is preferably one of the helicases shown in Table 4 or 5 of U.S. Patent Application No. 61/581,340 and International Application No. PCT/GB2012/053273 (published as WO 2012/098561) or a variant thereof. The XPD helicase most preferably comprises the sequence shown in SEQ ID NO: 52 or a variant thereof. SEQ ID NO: 52 is XPD Mbu (Methanococcoides burtonii; YP_566221.1; GI:91773529). XPD Mbu comprises YLWGTLSEG (Motif V; SEQ ID NO: 53) and QAMGRVVRSPTDYGARILLDGR (Motif VI; SEQ ID NO: 54).

A variant of a XPD helicase is an enzyme that has an amino acid sequence which varies from that of the wild-type helicase and which retains polynucleotide binding activity. In particular, a variant of SEQ ID NO: 52 is an enzyme that has an amino acid sequence which varies from that of SEQ ID NO: 52 and which retains polynucleotide binding activity. This can be measured as described above. The variant retains helicase activity. The variant must work in at least one of the two modes discussed below. Preferably, the variant works in both modes. The variant may include modifications that facilitate handling of the polynucleotide encoding the helicase and/or facilitate its activity at high salt concentrations and/or room temperature. Variants typically differ from the wild-type helicase in regions outside of XPD motifs V and VI discussed above. However, variants may include modifications within one or both of these motifs.

Over the entire length of the amino acid sequence of SEQ ID NO: 52, a variant will preferably be at least 10%, preferably 30% homologous to that sequence based on amino acid identity More preferably, the variant polypeptide may be at least 40%, at least 45%, at least 50%, at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 52 over the entire sequence. There may be at least 70%, for example at least 80%, at least 85%, at least 90% or at least 95%, amino acid identity over a stretch of 150 or more, for example 200, 300, 400, 500, 600, 700, 800, 900 or 1000 or more, contiguous amino acids (“hard homology”). Homology is determined as described above. The variant may differ from the wild-type sequence in any of the ways discussed above with reference to SEQ ID NOs: 2 and 4.

A variant of SEQ ID NO: 52 preferably comprises the XPD motif V and/or the XPD motif VI of the wild-type sequence A variant of SEQ ID NO. 52 more preferably comprises both XPD motifs V and VI of SEQ ID NO: 52 However, a variant of SEQ ID NO: 52 may comprise XPD motifs V and/or VI from a different wild-type sequence. For instance, a variant of SEQ ID NO: 52 may comprise any one of the preferred motifs shown in Table 5 of U.S. Patent Application No. 61/581,340 and International Application No. PCT/GB2012/053273 (published as WO 2012/098561). Variants of SEQ ID NO: 52 may also include modifications within XPD motif V and/or XPD motif VI of the wild-type sequence. Suitable modifications to these motifs are discussed above when defining the two motifs. A variant of SEQ ID NO: 52 preferably comprises one or more substituted cysteine residues and/or one or more substituted Faz residues to facilitate attachment as discussed above.

The helicase may be any of the modified helicases described and claimed in U.S. Provisional Application Nos. 61/673,446 and 61/673,452 (filed 19 Jul. 2012), US Provisional Application Nos. 61/774,694 and 61/774,862 (filed 8 Mar. 2013) and the two International Applications being filed concurrently with this application (Oxford Nanopore Refs: ONT IP 028 and ONT IP 033).

The helicase is more preferably a Hel308 helicase in which one or more cysteine residues and/or one or more non-natural amino acids have been introduced at one or more of the positions which correspond to D272, N273, D274, G281, E284, E285, E287, S288, T289, G290, E291, D293, T294, N300, R303, K304, N314, S315, N316, H317, R318, K319, L320, E322, R326, N328, S615, K717, Y720, N721 and S724 in Hel308 Mbu (SEQ ID NO: 10), wherein the helicase retains its ability to control the movement of a polynucleotide.

The Hel308 helicase preferably comprises a variant of one of SEQ ID NOs: 10, 13, 16 or 19 which comprises one or more cysteine residues and/or one or more non-natural amino acids at one or more of the positions which correspond to D272, N273, D274, G281, E284, E285, E287, S288, T289, G290, E291, D293, T294, N300, R303, K304, N314, S315, N316, H317, R318, K319, L320, E322, R326, N328, S615, K717, Y720, N721 and S724 in Hel308 Mbu (SEQ ID NO: 10).

The Hel308 helicase preferably comprises a variant of one of SEQ ID NOs: 10, 13, 16 or 19 which comprises one or more cysteine residues and/or one or more non-natural amino acids at one or more of the positions which correspond to D274, E284, E285, S288, S615, K717, Y720, E287, T289, G290, E291, N316 and K319 in Hel308 Mbu (SEQ ID NO: 10).

Tables 6a and 6b below show the positions in other Hel308 helicases which correspond to D274, E284, E285, S288, S615, K717, Y720, E287, T289, G290, E291, N316 and K319 in Hel308 Mbu (SEQ ID NO: 10). The lack of a corresponding position in another Hel308 helicase is marked as a “-”.

TABLE 6a
Positions which correspond to D274, E284, E285, S288,
S615, K717 and Y720 in Hel308 Mbu (SEQ lD NO: 10)

| SEQ | Hel308 | | | | | | | |
|---|---|---|---|---|---|---|---|---|
| ID NO: | homologue | A | B | C | D | E | F | G |
| 10 | Mbu | D274 | E284 | E285 | S288 | S615 | K717 | Y720 |
| 13 | Csy | D280 | K290 | I291 | S294 | P589 | T694 | N697 |
| 16 | Tga | L266 | S276 | L277 | Q280 | P583 | K689 | D692 |
| 19 | Mhu | S269 | Q277 | E278 | R281 | S583 | G685 | R688 |

TABLE 6b
Positions which correspond to E287, T289, G290, E291,
N316 and K319 in Hel308 Mbu (SEQ ID NO: 10).

| SEQ ID | Hel308 | | | | | | |
|---|---|---|---|---|---|---|---|
| NO: | homologue | H | I | J | K | L | M |
| 10 | Mbu | E287 | T289 | G290 | E291 | N316 | K319 |
| 13 | Csy | S293 | G295 | G296 | E297 | D322 | S325 |
| 16 | Tga | S279 | L281 | E282 | D283 | V308 | T311 |
| 19 | Mhu | R280 | L282 | R283 | D284 | Q309 | T312 |

The Hel308 helicase preferably comprises a variant of one of SEQ ID NOs: 10, 13, 16 or 19 which comprises one or more cysteine residues and/or one or more non-natural amino acids at one or more of the positions which correspond to D274, E284, E285, S288, S615, K717 and Y720 in Hel308 Mbu (SEQ ID NO: 10). The helicase may comprise one or more cysteine residues and/or one or more non-natural amino acids at any of the following combinations of the positions labelled A to G in each row of Table 6a: {A}, {B}, {C}, {D}, {G}, {E}, {F}, {A and B}, {A and C}, {A and D}, {A and G}, {A and E}, {A and F}, {B and C}, {B and D}, {B and G}, {B and E}, {B and F}, {C and D}, {C and G}, {C and E}, {C and F}, {D and G}, {D and E}, {D and F}, {G and E}, {G and F}, {E and F}, {A, B and C}, {A, B and D}, {A, B and G}, {A, B and E}, {A, B and F}, {A, C and D}, {A, C and G}, {A, C and E}, {A, C and F}, {A, D and G}, {A, D and E}, {A, D and F}, {A, G and E}, {A, G and F}, {A, E and F}, {B, C and D}, {B, C and G}, {B, C and E}, {B, C and F}, {B, D and G}, {B, D and E}, {B, D and F}, {B, G and E}, {B, G and F}, {B, E and F}, {C, D and G}, {C, D and E}, {C, D and F}, {C, G and E}, {C, G and F}, {C, E and F}, {D, G and E}, {D, G and F}, {D, E and F}, {G, E and F}, {A, B, C and D}, {A, B, C and G}, {A, B, C and E}, {A, B, C and F}, {A, B, D and G}, {A, B, D and E}, {A, B, D and F}, {A, B, G and E}, {A, B, G and F}, {A, B, E and F}, {A, C, D and G}, {A, C, D and E}, {A, C, D and F}, {A, C, G and E}, {A, C, G and F}, {A, C, E and F}, {A, D, G and E}, {A, D, G and F}, {A, D, E and F}, {A, G, E and F}, {B, C, D and G}, {B, C, D and E}, {B, C, D and F}, {B, C, G and E}, {B, C, G and F}, {B, C, E and F}, {B, D, G and E}, {B, D, G and F}, {B, D, E and F}, {B, G, E and F}, {C, D, G and E}, {C, D, G and F}, {C, D, E and F}, {C, G, E and F}, {D, G, E and F}, {A, B, C, D and G}, {A, B, C, D and E}, {A, B, C, D and F}, {A, B, C, G and E}, {A, B, C, G and F}, {A, B, C, E and F}, {A, B, D, G and E}, {A, B, D, G and F}, {A, B, D, E and F}, {A, B, G, E and F}, {A, C, D, G and E}, {A, C, D, G and F}, {A, C, D, E and F}, {A, C, G, E and F}, {A, D, G, E and F}, {B, C, D, G and E}, {B, C, D, G and F}, {B, C, D, E and F}, {B, C, G, E and F}, {B, D, G, E and F}, {C, D, G, E and F}, {A, B, C, D, G and E}, {A, B, C, D, G and F}, {A, B, C, D, E and F}, {A, B, C, G, E and F}, {A, B, D, G, E and F}. {A, C, D, G, E and F}, {B, C, D, G, E and F}, or {A, B, C, D, G, E and F}.

The Hel308 helicase more preferably comprises a variant of one of SEQ ID NOs: 10, 13, 16 or 19 which comprises one or more cysteine residues and/or one or more non-natural amino acids at one or more of the positions which correspond to D274, E284, E285, S288 and S615 in Hel308 Mbu (SEQ ID NO: 10).

In particular, the transport control protein may comprise a helicase dimer or a helicase multimer. A helicase multimer comprises two or more helicases attached together. The transport control protein may comprise two, three, four, five or more helicases. In other words, the transport control protein may comprise a helicase dimer, a helicase trimer, a helicase tetramer, a helicase pentamer and the like.

The two or more helicases can be attached together in any orientation. Identical or similar helicases may be attached via the same amino acid residue (i.e. same position) or spatially proximate amino acid residues (i.e. spatially proximate positions) in each helicase. This is termed the “head-to-head” formation. Alternatively, identical or similar helicases may be attached via amino acid residues (or positions) on opposite or different sides of each helicase. This is termed the “head-to-tail” formation. Helicase trimers comprising three identical or similar helicases may comprise both the head-to-head and head-to-tail formations.

The two or more helicases may be different from one another (i.e. the construct is a hetero-dimer, -trimer, -tetramer or -pentamer etc.). For instance, the transport control protein may comprise: (a) one or more Hel308 helicases and one or more XPD helicases; (b) one or more Hel308 helicases and one or more RecD helicases; (c) one or more Hel308 helicases and one or more TraI helicases; (d) one or more XPD helicases and one or more RecD helicases; (e) one or more XPD helicases and one or more TraI helicases; or (f) one or more RecD helicases and one or more TraI helicases. The transport control protein may comprise two different variants of the same helicase. For instance, the transport control protein may comprise two variants of one of the helicases discussed above with one or more cysteine residues or Faz residues introduced at different positions in each variant. In this instance, the helicases can be in a head-to-tail formation. In a preferred embodiment, a variant of SEQ ID NO: 10 comprising Q442C may be attached via cysteine linkage to a variant of SEQ ID NO: 10 comprising Q557C. Cys mutants of Hel308Mbu can also be made into hetero-dimers if necessary. In this approach, two different Cys mutant pairs such as Hel308Mbu-Q442C and Hel308Mbu-Q577C can be linked in head-to-tail fashion. Hetero-dimers can be formed in two possible ways. The first involves the use of a homo-bifunctional linker as discussed above. One of the helicase variants can be modified with a large excess of linker in such a way that one linker is attached to one molecule of the protein. This linker modified variant can then be purified away from unmodified proteins, possible homo-dimers and unreacted linkers to react with the other helicase variant. The resulting dimer can then be purified away from other species.

The second involves the use of hetero-bifunctional linkers. For example, one of the helicase variants can be modified with a first PEG linker containing maleimide or iodoacetamide functional group at one end and a cyclooctyne functional group (DIBO) at the other end. An example of this is shown below:

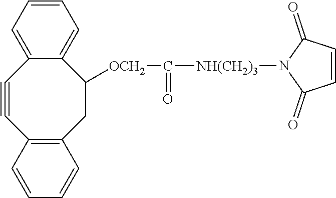

The second helicase variant can be modified with a second PEG linker containing maleimide or iodoacetamide functional group at one end and an azide functional group at the other end. An example is show below:

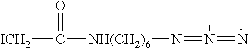

The two helicase variants with two different linkers can then be purified and clicked together (using Cu²⁻ free click chemistry) to make a dimer. Copper free click chemistry has been used in these applications because of its desirable properties. For example, it is fast, clean and not poisonous towards proteins. However, other suitable bio-orthogonal chemistries include, but are not limited to, Staudinger chemistry, hydrazine or hydrazide/aldehyde or ketone reagents (HyNic+4FB chemistry, including all Solulink™ reagents), Diels-Alder reagent pairs and boronic acid/salicyhydroxamate reagents.

Similar methodology may also be used for linking different Faz variants One Faz variant (such as SEQ ID NO: 10 comprising Q442C) can be modified with a large excess of linker in such a way that one linker is attached to one molecule of the protein. This linker modified Faz variant can then be purified away from unmodified proteins, possible homo-dimers and unreacted linkers to react with the second Faz variant (such as SEQ ID NO: 10 comprising Q577Faz). The resulting dimer can then be purified away from other species.

Hetero-dimers can also be made by linking cysteine variants and Faz variants of the same helicase or different helicases. For example, any of the above cysteine variants (such as SEQ ID NO: 10 comprising Q442C) can be used to make dimers with any of the above Faz variants (such SEQ ID NO: 10 comprising QS77Faz). Hetero-bifunctional PEG linkers with maleimide or iodoacetamide functionalities at one end and DBCO functionality at the other end can be used in this combination of mutants Δn example of such a linker is shown below (DBCO-PEG4-maleimide):

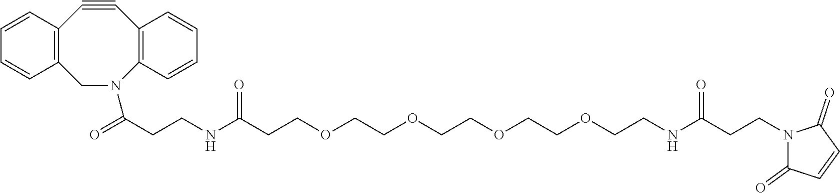 [SMILES: O=C(CCOCCOCCOCCOCCNC(=O)CCN1C(=O)C=CC1=O)NCCC(=O)N1Cc2ccccc2C#Cc2ccccc21]

The length of the linker can be varied by changing the number of PEG units between the two functional groups.

Helicase hetero-trimers can comprise three different types of helicases selected from Hel308 helicases, XPD helicases, RecD helicases, TraI helicases and variants thereof. The same is true for oligomers comprising more than three helicases. The two or more helicases may be different variants of the same helicase, such as different variants of SEQ ID NO: 10, 13, 16 or 19. The different variants may be modified at different positions to facilitate attachment via the different positions. The hetero-trimers may therefore be in a head-to-tail and head-to-headformation.

The two or more helicases may be the same as one another (i.e. the transport control protein is a homo-dimer, -trimer, -tetramer or -pentamer etc.) Homo-oligomers can comprise two or more Hel308 helicases, two or more XPD helicases, two or more RecD helicases, two or more TraI helicases or two or more of any of the variants discussed above. In such embodiments, the helicases are preferably attached using the same amino acid residue (i.e. same position) in each helicase. The helicases are therefore attached head-to-head. The helicases may be linked using a cysteine residue or a Faz residue that has been substituted into the helicases at the same position. Cysteine residues in identical helicase variants can be linked using a homo-bifunctional linker containing thiol reactive groups such as maleimide or iodoacetamide. These functional groups can be at the end of a polyethyleneglycol (PEG) chain as in the following example:

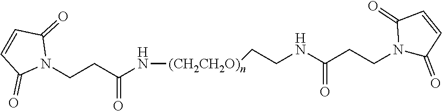

The length of the linker can be varied to suit the required applications. For example, n can be 2, 3, 4, 8, 11, 12, 16 or more. PEG linkers are suitable because they have favourable properties such as water solubility. Other non PEG linkers can also be used in cystein linkage.

By using similar approaches, identical Faz variants can also be made into homo-dimers. Homo-bifunctional linkers with DIBO functional groups can be used to link two molecules of the same Faz variant to make homo-dimers using Cu²⁺ free click chemistry. An example of a linker is given below:

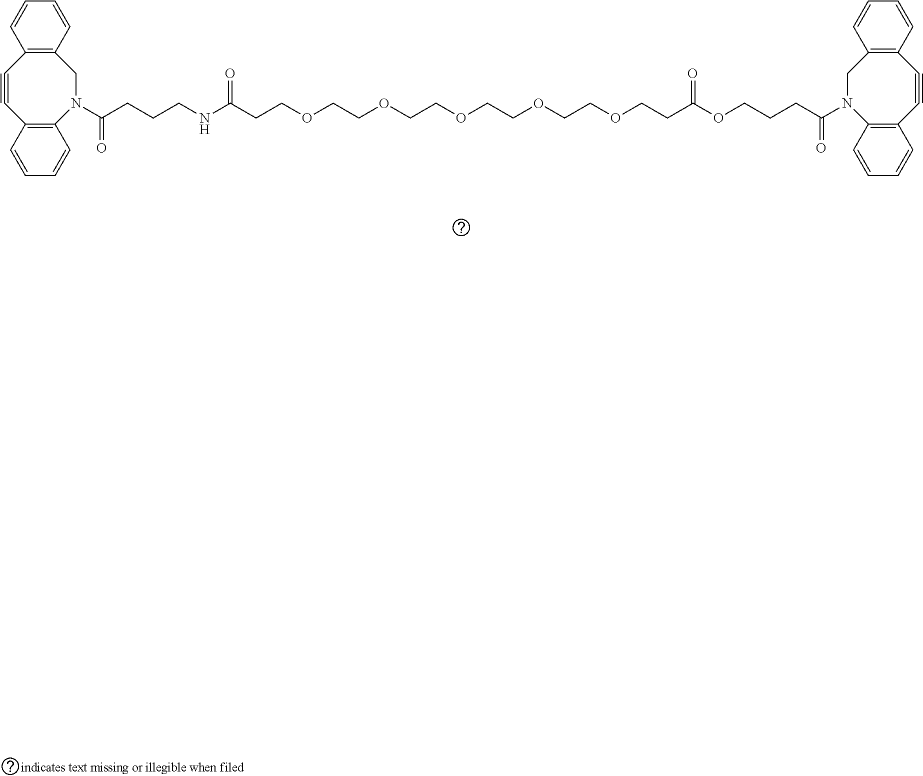 [SMILES: O=C(CCOCCOCCOCCOCCOCCC(=O)OCCCC(=O)N1Cc2ccccc2C#Cc2ccccc21)NCCCC(=O)N1Cc2ccccc2C#Cc2ccccc21]

The length of the PEG linker can vary to include 2, 4, 8, 12, 16 or more PEG units. Such linkers can also be made to incorporate a florescent tag to ease quantifications. Such fluorescence tags can also be incorporated into Maleimide linkers.

Preferred transport control proteins of the invention are shown in the Table 7 below.

TABLE 7
Preferred transport control proteins of the invention
Hel308Mbu-A700C dimer 2 kDa
Hel308Mbu-A700C dimer 3.4 kDa
Hel308Mbu-Q442C 2 kDa linker homodimer
Hel308Mbu-Q442C 3.4 kDa linker homodimer
Hel308Mbu-A700C 2 kDa linker homodimer
Hel308Mbu-A700C-strepII. 2 kDa PEG homodimer
MspA dimer treated with proteaseK lower band
MspA dimer treated with proteaseK upper band
MspA dimer treated with proteaseK + heat treatment lower band
MspA dimer treated with proteaseK + heat treatment upper band
Hel308Mhu-WT 2 kDa Dimer
Helicase 2k dimer (Hel308Mbu R681A, R687A, A700C-STrEP)
Helicase 2k dimer (Hel308Mbu R687A, A700C-STrEP)
Hel308Mhu-WT 2 kDa Dimer
Hel308Tga N674C Dimer 2 kDa
Hel308Tga N674C Dimer 2 kDa tests for assay
Hel308 Tga-R657A-N674C-STrEP Dimer 2 kDa

The transport control protein may be a polynucleotide binding domain derived from a helicase. For instance, the transport control protein preferably comprises the sequence shown in SEQ ID NO: 61 or 62 or a variant thereof. A variant of SEQ ID NO: 61 or 62 is a protein that has an amino acid sequence which varies from that of SEQ ID NO: 61 or 62 and which retains polynucleotide binding activity. This can be measured as described above. The variant may include modifications that facilitate binding of the polynucleotide and/or facilitate its activity at high salt concentrations and/or room temperature.

Over the entire length of the amino acid sequence of SEQ ID NO: 61 or 62, a variant will preferably be at least 50% homologous to that sequence based on amino acid identity. More preferably, the variant polypeptide may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 61 or 62 over the entire sequence. There may be at least 80%, for example at least 85%, 90% or 95%, amino acid identity over a stretch of 40 or more, for example 50, 60, 70 or 80 or more, contiguous amino acids (“hard homology”). Homology is determined as described above. The variant may differ from the wild-type sequence in any of the ways discussed below with reference to SEQ ID NOs: 2 and 4.

The topoisomerase is preferably a member of any of the Moiety Classification (EC) groups 5.99.1.2 and 5.99.1.3.

The transport control protein may be any of the enzymes discussed above.

The transport control protein may be labelled with a revealing label. The label may be any of those described above.

The transport control protein may be isolated from any protein-producing organism, such as E. coli, T. thermophilus or bacteriophage, or made synthetically or by recombinant means. For example, the transport control protein may be synthesized by in vitro translation and transcription as described below. The transport control protein may be produced in large scale following purification as described above.

The SSB is preferably attached to the transport control protein such that the resulting construct has the ability to control the movement of the target polynucleotide. Such a construct is a useful tool for controlling the movement of a polynucleotide during Strand Sequencing. A problem which occurs in sequencing polynucleotides, particularly those of 500 nucleotides or more, is that the molecular motor which is controlling translocation of the polynucleotide may disengage from the polynucleotide. This allows the polynucleotide to be pulled through the pore rapidly and in an uncontrolled manner in the direction of the applied field. The construct is less likely to disengage from the polynucleotide being sequenced. The construct can provide increased read lengths of the polynucleotide as it controls the translocation of the polynucleotide through a nanopore. The ability to translocate an entire polynucleotide through a nanopore under the control of the construct described above allows characteristics of the polynucleotide, such as its sequence, to be estimated with improved accuracy and speed over known methods. This becomes more important as strand lengths increase and molecular motors are required with improved processivity. The construct is particularly effective in controlling the translocation of target polynucleotides of 500 nucleotides or more, for example 1000 nucleotides, 5000, 10000, 20000, 50000, 100000 or more.

The construct has the ability to control the movement of a polynucleotide. The ability of a construct to control the movement of a polynucleotide can be assayed using any method known in the art. For instance, the construct may be contacted with a polynucleotide and the position of the polynucleotide may be determined using standard methods. The ability of a construct to control the movement of a polynucleotide is typically assayed as described in the Examples.

The construct may be isolated, substantially isolated, purified or substantially purified. A construct is isolated or purified if it is completely free of any other components, such as lipids, polynucleotides or pore monomers. A construct is substantially isolated if it is mixed with carriers or diluents which will not interfere with its intended use. For instance, a construct is substantially isolated or substantially purified if it is present in a form that comprises less than 10%, less than 5%, less than 2% or less than 1% of other components, such as lipids, polynucleotides or pore monomers.

In the construct, the transport control protein, such as the helicase, is attached to the SSB. The transport control protein is preferably covalently attached to the SSB. The transport control protein may be attached to the SSB at more than one, such as two or three, points.

The transport control protein can be covalently attached to the SSB using any method known in the art. The transport control protein and SSB may be produced separately and then attached together. The two components may be attached in any configuration. For instance, they may be attached via their terminal (i.e. amino or carboxy terminal) amino acids. Suitable configurations include, but are not limited to, the amino terminus of the SSB being attached to the carboxy terminus of the transport control protein and vice versa. Alternatively, the two components may be attached via amino acids within their sequences. For instance, the SSB may be attached to one or more amino acids in a loop region of the transport control protein. In a preferred embodiment, terminal amino acids of the SSB are attached to one or more amino acids in the loop region of a transport control protein. Terminal amino acids and loop regions can be identified using methods known in the art (Edman P., Acta Chemica Scandinavia, (1950), 283-293). For instance, loop regions can be identified using protein modeling. This exploits the fact that protein structures are more conserved than protein sequences amongst homologues. Hence, producing atomic resolution models of proteins is dependent upon the identification of one or more protein structures that are likely to resemble the structure of the query sequence. In order to assess whether a suitable protein structure exists to use as a “template” to build a protein model, a search is performed on the protein data bank (PDB) database. A protein structure is considered a suitable template if it shares a reasonable level of sequence identity with the query sequence. If such a template exists, then the template sequence is “aligned” with the query sequence, i.e. residues in the query sequence are mapped onto the template residues. The sequence alignment and template structure are then used to produce a structural model of the query sequence. Hence, the quality of a protein model is dependent upon the quality of the sequence alignment and the template structure.

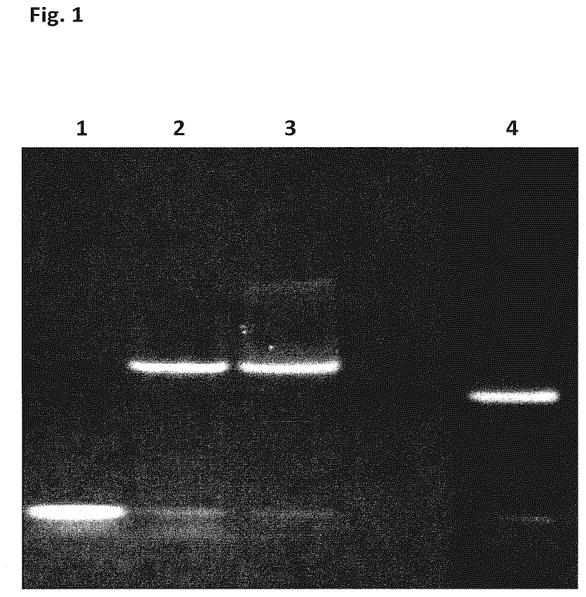

The two components may be attached via their naturally occurring amino acids, such as cysteines, threonines, serines, aspartates, asparagines, glutamates and glutamines. Naturally occurring amino acids may be modified to facilitate attachment. For instance, the naturally occurring amino acids may be modified by acylation, phosphorylation, glycosylation or farnesylation. Other suitable modifications are known in the art. Modifications to naturally occurring amino acids may be post-translation modifications. The two components may be attached via amino acids that have been introduced into their sequences. Such amino acids are preferably introduced by substitution. The introduced amino acid may be cysteine or a non-natural amino acid that facilitates attachment. Suitable non-natural amino acids include, but are not limited to, 4-azido-L-phenylalanine (Faz), and any one of the amino acids numbered 1-71 included in FIG. 1 of Liu C. C. and Schultz P. G., Annu. Rev. Biochem, 2010, 79, 413-444. The introduced amino acids may be modified as discussed above.

In a preferred embodiment, the transport control protein is chemically attached to the SSB, for instance via a linker molecule. Linker molecules are discussed in more detail below. One suitable method of chemical attachment is cysteine linkage. This is discussed in more detail below.

The transport control protein may be transiently attached to the SSB by a hexa-his tag or Ni-NTA. The transport control protein and SSB may also be modified such that they transiently attach to each other.

In another preferred embodiment, the transport control protein is genetically fused to the SSB. A transport control protein is genetically fused to a SSB if the whole construct is expressed from a single polynucleotide sequence. The coding sequences of the transport control protein and SSB may be combined in any way to form a single polynucleotide sequence encoding the construct. Genetic fusion of a pore to a nucleic acid binding protein is discussed in International Application No. PCT/GB09/001679 (published as WO 2010/004265).

The transport control protein and SSB may be genetically fused in any configuration. The transport control protein and SSB may be fused via their terminal amino acids. For instance, the amino terminus of the SSB may be fused to the carboxy terminus of the transport control protein and vice versa. The amino acid sequence of the SSB is preferably added in frame into the amino acid sequence of the transport control protein. In other words, the SSB is preferably inserted within the sequence of the transport control protein. In such embodiments, the transport control protein and SSB are typically attached at two points, i.e. via the amino and carboxy terminal amino acids of the SSB. If the SSB is inserted within the sequence of the transport control protein, it is preferred that the amino and carboxy terminal amino acids of the SSB are in close proximity and are each attached to adjacent amino acids in the sequence of the transport control protein or variant thereof. In a preferred embodiment, the SSB is inserted into a loop region of the transport control protein.

The construct retains the ability of the transport control protein to control the movement of a polynucleotide. This ability of the transport control protein is typically provided by its three dimensional structure that is typically provided by its β-strands and α-helices. The α-helices and β-strands are typically connected by loop regions. In order to avoid affecting the ability of the transport control protein to control the movement of a polynucleotide, the SSB is preferably genetically fused to either end of the transport control protein or inserted into a surface-exposed loop region of the transport control protein. The loop regions of specific transport control proteins can be identified using methods known in the art. For instance, the loop regions can be identified using protein modelling, x-ray diffraction measurement of the protein in a crystalline state (Rupp B (2009). Biomolecular Crystallography: Principles, Practice and Application to Structural Biology. New York: Garland Science.), nuclear magnetic resonance (NMR) spectroscopy of the protein in solution (Mark Rance; Cavanagh, John; Wayne J. Fairbrother; Arthur W. Hunt III; Skelton, NNicholas J. (2007). Protein NMR spectroscopy: principles and practice (2nd ed.). Boston: Academic Press.) or cryo-electron microscopy of the protein in a frozen-hydrated state (van Heel M, Gowen B, Matadeen R, Orlova E V, Finn R, Pape T, Cohen D, Stark H, Schmidt R, Schatz M, Patwardhan A (2000). “Single-particle electron cryo-microscopy: towards atomic resolution.”. Q Rev Biophys. 33:307-69. Structural information of proteins determined by above mentioned methods are publicly available from the protein bank (PDB) database.

For Hel308 helicases (SEQ ID NOs: 10, 13, 16 and 19), β-strands can only be found in the two RecA-like engine domains (domains 1 and 2). These domains are responsible for coupling the hydrolysis of the fuel nucleotide (normally ATP) with movement. The important domains for ratcheting along a polynucleotide are domains 3 and 4, but above all domain 4. Interestingly, both of domains 3 and 4 comprise only α-helices. There is an important α-helix in domain 4 called the ratchet helix. As a result, in the Hel308 embodiments of the invention, the SSB is preferably not genetically fused to any of the α-helixes.

The transport control protein may be attached directly to the SSB. The transport control protein is preferably attached to the SSB using one or more, such as two or three, linkers. The one or more linkers may be designed to constrain the mobility of the SSB The linkers may be attached to one or more reactive cysteine residues, reactive lysine residues or non-natural amino acids in the transport control protein and/or SSB. The non-natural amino acid may be any of those discussed above. The non-natural amino acid is preferably 4-azido-L-phenylalanine (Faz). Suitable linkers are well-known in the art.

The transport control protein is preferably attached to the SSB using one or more chemical crosslinkers or one or more peptide linkers. Suitable chemical crosslinkers are well-known in the art. Suitable chemical crosslinkers include, but are not limited to, those including the following functional groups: maleimide, active esters, succinimide, azide, alkyne (such as dibenzocyclooctynol (DIBO or DBCO), difluoro cycloalkynes and linear alkynes), phosphine (such as those used in traceless and non-traceless Staudinger ligations), haloacetyl (such as iodoacetamide), phosgene type reagents, sulphonyl chloride reagents, isothiocyanates, acyl halides, hydrazines, disulphides, vinyl sulfones, aziridines and photoreactive reagents (such as aryl azides, diaziridines).

Reactions between amino acids and functional groups may be spontaneous, such as cysteine/maleimide, or may require external reagents, such as Cu(I) for linking azide and linear alkynes.

Linkers can comprise any molecule that stretches across the distance required. Linkers can vary in length from one carbon (phosgene-type linkers) to many Angstroms. Examples of linear molecules, include but are not limited to, are polyethyleneglycols (PEGs), polypeptides, polysaccharides, deoxyribonucleic acid (DNA), peptide nucleic acid (PNA), threose nucleic acid (TNA), glycerol nucleic acid (GNA), saturated and unsaturated hydrocarbons, polyamides. These linkers may be inert or reactive, in particular they may be chemically cleavable at a defined position, or may be themselves modified with a fluorophore or ligand. The linker is preferably resistant to dithiothreitol (DTT).

Cleavable linkers can be used as an aid to separation of constructs from non-attached components and can be used to further control the synthesis reaction. For example, a hetero-bifunctional linker may react with the transport control protein, but not the SSB. If the free end of the linker can be used to bind the transport control protein to a surface, the unreacted transport control proteins from the first reaction can be removed from the mixture. Subsequently, the linker can be cleaved to expose a group that reacts with the SSB. In addition, by following this sequence of linkage reactions, conditions may be optimised first for the reaction to the transport control protein, then for the reaction to the SSB after cleavage of the linker. The second reaction would also be much more directed towards the correct site of reaction with the SSB because the linker would be confined to the region to which it is already attached.

Preferred crosslinkers include 2,5-dioxopyrrolidin-1-yl 3-(pyridin-2-yldisulfanyl) propanoate, 2,5-dioxopyrrolidin-1-yl 4-(pyridin-2-yldisulfanyl) butanoate and 2,5-dioxopyrrolidin-1-yl 8-(pyridin-2-yldisulfanyl)octananoate. The most preferred crosslinkers are succinimidyl 3-(2-pyridyldithio) propionate (SPDP) and maleimide-PEG (2 kDa)-maleimide (alpha, omega-bis-maleimido poly(ethylene glycol)).

The transport control protein may be covalently attached to the bifunctional crosslinker before the transport control protein/crosslinker complex is covalently attached to the SSB. Alternatively, the SSB may be covalently attached to the bifunctional crosslinker before the bifunctional crosslinker/SSB complex is attached to the transport control protein. The transport control protein and SSB may be covalently attached to the chemical crosslinker at the same time.

The transport control protein may be attached to the SSB using two different linkers that are specific for each other. One of the linkers is attached to the transport control protein and the other is attached to the SSB. Once mixed together, the linkers should react to form a construct. The transport control protein may be attached to the SSB using the hybridization linkers described in International Application No. PCT/GB10/000132 (published as WO 2010/086602) In particular, the transport control protein may be attached to the SSB using two or more linkers each comprising a hybridizable region and a group capable of forming a covalent bond. The hybridizable regions in the linkers hybridize and link the transport control protein and the SSB. The linked transport control protein and the SSB are then coupled via the formation of covalent bonds between the groups. Any of the specific linkers disclosed in International Application No. PCT/GB10/000132 (published as WO 2010/086602) may be used in accordance with the invention.

The transport control protein and the SSB may be modified and then attached using a chemical crosslinker that is specific for the two modifications. Any of the crosslinkers discussed above may be used.

Alternatively, the linkers preferably comprise amino acid sequences. Such linkers are peptide linkers. The length, flexibility and hydrophilicity of the peptide linker are typically designed such that it does not to disturb the functions of the transport control protein and SSB. Preferred flexible peptide linkers are stretches of 2 to 20, such as 4, 6, 8, 10 or 16, serine and/or glycine amino acids. More preferred flexible linkers include (SG)₁, (SG)₂, (SG)₃, (SG)₄, (SG)₅, (SG)₈, (SG)₁₀, (SG)₁₅ or (SG)₂₀ wherein S is serine and G is glycine. Preferred rigid linkers are stretches of 2 to 30, such as 4, 6, 8, 16 or 24, proline amino acids. More preferred rigid linkers include (P)₁₂, wherein P is proline.

The linkers may be labelled. Suitable labels include, but are not limited to, fluorescent molecules (such as Cy3 or AlexaFluor®555), radioisotopes, e.g. ¹²⁵I, ³⁵S, enzymes, antibodies, antigens, polynucleotides and ligands such as biotin. Such labels allow the amount of linker to be quantified. The label could also be a cleavable purification tag, such as biotin, or a specific sequence to show up in an identification method, such as a peptide that is not present in the protein itself, but that is released by trypsin digestion.

A preferred method of attaching the transport control protein to the SSB is via cysteine linkage. This can be mediated by a bi-functional chemical linker or by a polypeptide linker with a terminal presented cysteine residue. Linkage can occur via natural cysteines in the transport control protein and/or SSB. Alternatively, cysteines can be introduced into the transport control protein and/or SSB. If the transport control protein is attached to the SSB via cysteine linkage, the one or more cysteines have preferably been introduced to the transport control protein and/or SSB by substitution.

The length, reactivity, specificity, rigidity and solubility of any bi-functional linker may be designed to ensure that the SSB is positioned correctly in relation to the transport control protein and the function of both the transport control protein and SSB is retained. Suitable linkers include bismaleimide crosslinkers, such as 1,4-bis(maleimido)butane (BMB) or bis(maleimido)hexane. One draw back of bi-functional linkers is the requirement of the transport control protein and SSB to contain no further surface accessible cysteine residues if attachment at specific sites is preferred, as binding of the bi-functional linker to surface accessible cysteine residues may be difficult to control and may affect substrate binding or activity. If the transport control protein and/or SSB does contain several accessible cysteine residues, modification of the transport control protein and/or SSB may be required to remove them while ensuring the modifications do not affect the folding or activity of the transport control protein and SSB. This is discussed in International Application No. PCT/GB10/000133 (published as WO 2010/086603). In a preferred embodiment, a reactive cysteine is presented on a peptide linker that is genetically attached to the SSB. This means that additional modifications will not necessarily be needed to remove other accessible cysteine residues from the SSB. The reactivity of cysteine residues may be enhanced by modification of the adjacent residues, for example on a peptide linker. For instance, the basic groups of flanking arginine, histidine or lysine residues will change the pKa of the cysteines thiol group to that of the more reactive S″ group. The reactivity of cysteine residues may be protected by thiol protective groups such as 5,5′-dithiobis-(2-nitrobenzoic acid) (dTNB). These may be reacted with one or more cysteine residues of the SSB or transport control protein, either as a monomer or part of an oligomer, before a linker is attached. Selective deprotection of surface accessible cysteines may be possible using reducing reagents immobilized on beads (for example immobilized tris(2-carboxyethyl)phosphine, TCEP).

Another preferred method of attaching the transport control protein to the SSB is via 4-azido-L-phenylalanine (Faz) linkage. This can be mediated by a bi-functional chemical linker or by a polypeptide linker with a terminal presented Faz residue. The one or more Faz residues have preferably been introduced to the transport control protein and/or SSB by substitution.

Cross-linkage of transport control proteins or SSB to themselves may be prevented by keeping the concentration of linker in a vast excess of the transport control protein and/or SSB. Alternatively, a “lock and key” arrangement may be used in which two linkers are used. Only one end of each linker may react together to form a longer linker and the other ends of the linker each react with a different part of the construct (i.e. transport control protein or SSB). This is discussed in more detail below.

The site of attachment is selected such that, when the construct is contacted with a polynucleotide, both the transport control protein and the SSB can bind to the polynucleotide and control its movement.

Attachment can be facilitated using the polynucleotide binding activities of the transport control protein and the SSB. For instance, complementary polynucleotides can be used to bring the transport control protein and SSB together as they hybridize. The transport control protein can be bound to one polynucleotide and the SSB can be bound to the complementary polynucleotide. The two polynucleotides can then be allowed to hybridise to each other. This will bring the transport control protein into close contact with the SSB, making the linking reaction more efficient. This is especially helpful for attaching two or more transport control proteins in the correct orientation for controlling movement of a target polynucleotide. An example of complementary polynucleotides that may be used are shown below.

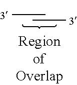

Tags can be added to the construct to make purification of the construct easier. These tags can then be chemically or enzymatically cleaved off, if their removal is necessary. Fluorophores or chromophores can also be included, and these could also be cleavable.

A simple way to purify the construct is to include a different purification tag on each protein (i.e. the transport control protein and the SSB), such as a hexa-His-tag and a Strep-tag®. If the two proteins are different from one another, this method is particularly useful. The use of two tags enables only the species with both tags to be purified easily.

If the two proteins do not have two different tags, other methods may be used. For instance, proteins with free surface cysteines or proteins with linkers attached that have not reacted to form a construct could be removed, for instance using an iodoacetamide resin for maleimide linkers.

Constructs can also be purified from unreacted proteins on the basis of a different DNA processivity property. In particular, a construct can be purified from unreacted proteins on the basis of an increased affinity for a polynucleotide, a reduced likelihood of disengaging from a polynucleotide once bound and/or an increased read length of a polynucleotide as it controls the translocation of the polynucleotide through a nanopore.

The invention provides a construct comprising at least one helicase and an SSB as described above, wherein the helicase is attached to the SSB and the construct has the ability to control the movement of a polynucleotide. The construct may comprise two or more helicases, such as three, four, five or more helicases. The construct may comprise any of the helicases described above. Any of the discussion concerning attaching a transport control protein to an SSB equally applies to this embodiment.

### Strand Sequencing

In a preferred embodiment, the method comprises:
(a) contacting the target polynucleotide with a transmembrane pore and a SSB as defined above such that the target polynucleotide moves through the pore and the SSB does not move through the pore, and
(b) measuring the current passing through the pore as the polynucleotide moves with respect to the pore wherein the current is indicative of one or more characteristics of the target polynucleotide and thereby characterising the target polynucleotide. The target polynucleotide is preferably contacted with the pore and the SSB on the same side of the membrane.

The methods may be carried out using any apparatus that is suitable for investigating a membrane/pore system in which a pore is present in a membrane. The method may be carried out using any apparatus that is suitable for transmembrane pore sensing. For example, the apparatus comprises a chamber comprising an aqueous solution and a barrier that separates the chamber into two sections. The barrier typically has an aperture in which the membrane containing the pore is formed. Alternatively the barrier forms the membrane in which the pore is present.

The methods may be carried out using the apparatus described in International Application No. PCT/GB08/000562 (WO 2008/102120).

The methods may involve measuring the current passing through the pore as the polynucleotide moves with respect to the pore. Therefore the apparatus may also comprise an electrical circuit capable of applying a potential and measuring an electrical signal across the membrane and pore. The methods may be carried out using a patch clamp or a voltage clamp. The methods preferably involve the use of a voltage clamp.

The methods of the invention may involve the measuring of a current passing through the pore as the polynucleotide moves with respect to the pore. Suitable conditions for measuring ionic currents through transmembrane protein pores are known in the art and disclosed in the Example. The method is typically carried out with a voltage applied across the membrane and pore. The voltage used is typically from +2 V to −2 V, typically −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 100 mV to 240 mV and most preferably in the range of 120 mV to 220 mV. It is possible to increase discrimination between different nucleotides by a pore by using an increased applied potential.

The methods are typically carried out in the presence of any charge carriers, such as metal salts, for example alkali metal salt, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In the exemplary apparatus discussed above, the salt is present in the aqueous solution in the chamber. Potassium chloride (KCl), sodium chloride (NaCl), caesium chloride (CsCl) or a mixture of potassium ferrocyanide and potassium ferricyanide is typically used KCl, NaCl and a mixture of potassium ferrocyanide and potassium ferricyanide are preferred. The salt concentration may be at saturation. The salt concentration may be 3 M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is preferably from 150 mM to 1 M. Hel308, XPD, RecD and TraI helicases surprisingly work under high salt concentrations. The method is preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of the presence of a nucleotide to be identified against the background of normal current fluctuations.

The methods are typically carried out in the presence of a buffer. In the exemplary apparatus discussed above, the buffer is present in the aqueous solution in the chamber. Any buffer may be used in the method of the invention. Typically, the buffer is HEPES Another suitable buffer is Tris-HCl buffer. The methods are typically carried out at a pH of from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 7.5.

The methods may be carried out at from 0° C. to 100° C., from 15° C. to 95° C., from 16° C. to 90° C., from 17° C. to 85° C., from 18° C. to 80° C., 19° C. to 70° C., or from 20° C. to 60° C. The methods are typically carried out at room temperature. The methods are optionally carried out at a temperature that supports enzyme function, such as about 37° C.

The method may be carried out in the presence of free nucleotides or free nucleotide analogues and/or an enzyme cofactor that facilitates the action of the transport control protein.

The method may also be carried out in the absence of free nucleotides or free nucleotide analogues and in the absence of an enzyme cofactor The free nucleotides may be one or more of any of the individual nucleotides discussed above. The free nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), cyclic adenosine monophosphate (CAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP). The free nucleotides are preferably selected from AMP, TMP, GMP, CMP, UMP, dAMP, dTMP, dGMP or dCMP. The free nucleotides are preferably adenosine triphosphate (ATP). The enzyme cofactor is a factor that allows the transport control protein to function. The enzyme cofactor is preferably a divalent metal cation. The divalent metal cation is preferably Mg²⁺, Mn²⁺, Ca²⁺ or Co²⁺. The enzyme cofactor is most preferably Mg²⁺.

The target polynucleotide may be contacted with the SSB and the pore in any order. It is preferred that, when the target polynucleotide is contacted with the SSB and the pore, the target polynucleotide firstly forms a complex with the SSB. When the voltage is applied across the pore, the target polynucleotide/SSB complex then forms a complex with the pore and controls the movement of the polynucleotide through the pore.

As discussed above, helicases may work in two modes with respect to the pore. The constructs of the invention comprising such helicases can also work in two mode. First, the method is preferably carried out using the construct such that it moves the target sequence through the pore with the field resulting from the applied voltage. In this mode the 5′ end of the DNA is first captured in the pore, and the construct moves the DNA into the pore such that the target sequence is passed through the pore with the field until it finally translocates through to the trans side of the bilayer. Alternatively, the method is preferably carried out such that the construct moves the target sequence through the pore against the field resulting from the applied voltage. In this mode the 3′ end of the DNA is first captured in the pore, and the construct moves the DNA through the pore such that the target sequence is pulled out of the pore against the applied field until finally ejected back to the cis side of the bilayer.

### Polynucleotide Sequences

Any of the proteins described herein may be expressed using methods known in the art. Polynucleotide sequences may be isolated and replicated using standard methods in the art. Chromosomal DNA may be extracted from a helicase producing organism, such as Methanococcoides burtonii, and/or a SSB producing organism, such as E. coli. The gene encoding the sequence of interest may be amplified using PCR involving specific primers. The amplified sequences may then be incorporated into a recombinant replicable vector such as a cloning vector. The vector may be used to replicate the polynucleotide in a compatible host cell. Thus polynucleotide sequences may be made by introducing a polynucleotide encoding the sequence of interest into a replicable vector, introducing the vector into a compatible host cell, and growing the host cell under conditions which bring about replication of the vector. The vector may be recovered from the host cell. Suitable host cells for cloning of polynucleotides are known in the art and described in more detail below.

The polynucleotide sequence may be cloned into a suitable expression vector. In an expression vector, the polynucleotide sequence is typically operably linked to a control sequence which is capable of providing for the expression of the coding sequence by the host cell. Such expression vectors can be used to express a construct.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences Multiple copies of the same or different polynucleotide may be introduced into the vector.

The expression vector may then be introduced into a suitable host cell. Thus, a construct can be produced by inserting a polynucleotide sequence encoding a construct into an expression vector, introducing the vector into a compatible bacterial host cell, and growing the host cell under conditions which bring about expression of the polynucleotide sequence.

The vectors may be for example, plasmid, virus or phage vectors provided with an origin of replication, optionally a promoter for the expression of the said polynucleotide sequence and optionally a regulator of the promoter. The vectors may contain one or more selectable marker genes, for example an ampicillin resistance gene. Promoters and other expression regulation signals may be selected to be compatible with the host cell for which the expression vector is designed. A T7, trc, lac, ara or λ_{L} promoter is typically used.

The host cell typically expresses the construct at a high level. Host cells transformed with a polynucleotide sequence will be chosen to be compatible with the expression vector used to transform the cell. The host cell is typically bacterial and preferably E. coli. Any cell with a λ DE3 lysogen, for example C41 (DE3), BL21 (DE3), JM109 (DE3), B834 (DE3), TUNER, Origami and Origami B, can express a vector comprising the T7 promoter.

### Other Methods

The invention also provides a method of forming a sensor for characterising a target polynucleotide. The method comprises forming a complex between a pore and a SSB as described above. The complex may be formed by contacting the pore and the SSB in the presence of the target polynucleotide and then applying a potential across the pore. The applied potential may be a chemical potential or a voltage potential as described above.

Alternatively, the complex may be formed by covalently attaching the pore to the SSB. Methods for covalent attachment are known in the art and disclosed, for example, in International Application Nos. PCT/GB09/001679 (published as WO 2010/004265) and PCT/GB10/000133 (published as WO 2010/086603). Methods are also discussed above with reference to attaching the SSB to the transport control protein. The complex is a sensor for characterising the target polynucleotide. The method preferably comprises forming a complex between a pore derived from Msp and a SSB. Any of the embodiments discussed above with reference to the methods of the invention equally apply to this method. The invention also provides a sensor produced using the method of the invention

### Kits

The present invention also provides a kit for characterising a target polynucleotide. The kit comprises (a) a pore and (b) a SSB as described above. Any of the embodiments discussed above with reference to the method of the invention equally apply to the kits.

The kit may further comprise the components of a membrane, such as the phospholipids needed to form an amphiphilic layer, such as a lipid bilayer.

The kit of the invention may additionally comprise one or more other reagents or instruments which enable any of the embodiments mentioned above to be carried out. Such reagents or instruments include one or more of the following: suitable buffer(s) (aqueous solutions), means to obtain a sample from a subject (such as a vessel or an instrument comprising a needle), means to amplify and/or express polynucleotides, a membrane as defined above or voltage or patch clamp apparatus. Reagents may be present in the kit in a dry state such that a fluid sample resuspends the reagents. The kit may also, optionally, comprise instructions to enable the kit to be used in the method of the invention or details regarding which patients the method may be used for. The kit may, optionally, comprise nucleotides

### Apparatus

The invention also provides an apparatus for characterising a target polynucleotide. The apparatus comprises a plurality of pores and a plurality of SSBs as described above. The apparatus preferably further comprises instructions for carrying out the method of the invention. The apparatus may be any conventional apparatus for polynucleotide analysis, such as an array or a chip. Any of the embodiments discussed above with reference to the methods of the invention are equally applicable to the apparatus of the invention.

The apparatus is preferably set up to carry out the method of the invention.

The apparatus preferably comprises:
a sensor device that is capable of supporting the plurality of pores and being operable to perform polynucleotide characterisation using the pores and SSBs; and
at least one reservoir for holding material for performing the characterisation.

The apparatus preferably comprises:
a sensor device that is capable of supporting the membrane and plurality of pores and being operable to perform polynucleotide characterising using the pores and SSBs as described above;
at least one reservoir for holding material for performing the characterising;
a fluidics system configured to controllably supply material from the at least one reservoir to the sensor device; and
one or more containers for receiving respective samples, the fluidics system being configured to supply the samples selectively from the one or more containers to the sensor device. The apparatus may be any of those described in International Application No. PCT/GB08/004127 (published as WO 2009/077734), PCT/GB10/000789 (published as WO 2010/122293), International Application No. PCT/GB10/002206 (not yet published) or International Application No. PCT/US99/25679 (published as WO 00/28312).

### Methods of Producing Constructs of the Invention

The invention also provides a method of producing a construct of the invention. The method comprises attaching, preferably covalently attaching, an SSB as defined above to at least one helicase. Any of the helicases and SSBs discussed above can be used in the methods. The site of and method of attachment are selected as discussed above.

The method preferably further comprises determining whether or not the construct is capable of controlling the movement of a polynucleotide. Assays for doing this are described above. If the movement of a polynucleotide can be controlled, the helicase and SSB have been attached correctly and a construct of the invention has been produced. If the movement of a polynucleotide cannot be controlled, a construct of the invention has not been produced. The following Example illustrates the invention.

### Example 1—Expression and Purification of EcoSSB-WT (SEQ ID NO: 65) and EcoSSB Mutants (SEQ ID NO's: 66-69)

All proteins were expressed with an N-terminal hexahistidine tag and TEV protease digestion site in BL21 STAR (DE3) competent cells (Invitrogen). Transformed colonies from LB-agar plates with 100 μg/ml ampicillin were grown in TB media with 100 μg/ml ampicillin and 20 μg/ml chloramphenicol at 37° C. for 7 h until OD600 reached 1.5 for EcoSSB-WT (SEQ ID NO: 65), EcoSSB-CterAla (SEQ ID) NO: 66) and EcoSSB-NGGN (SEQ ID NO: 67) and 0.15 for EcoSSB-Q152del (SEQ ID NO: 68) and EcoSSB-G117del (SEQ ID NO 69) (slow growth may be due to high toxicity of these mutants). Cultures were moved to 18° C. and allowed to cool for 30 mins before isopropyl B-D-1-thiogalactopyranoside (IPTG) was added to a final concentration of 1 mM and fermentation continued overnight (16-18 h). Cells were harvested by centrifugation at 4000 g and pellets were lysed for 2 h at 4° C. in a buffer containing 1× BugBuster (Novagen), 50 mM TrisHCl pH 8.0, 500 mM NaCl, 20 mM imidazole and 5% (w/v) glycerol, protease inhibitors (Calbiochem Protease Inhibitor Cocktail set V) and Benzonase nuclease (Sigma). The lysate was then centrifuged and filtered through 0.22 μm filters before loading onto HisTrapFF crude columns (GE Healthcare) equilibrated in buffer A (50 mM TrisHCl pH 8.0, 500 mM NaCl, 20 mM imidazole, 5% (w/v) glycerol) After loading, the column was washed for 20 column volumes (CV) with buffer A and 20 CV with buffer W (50 mM TrisHCl pH 8.0, 1000 mM NaCl, 40 mM imidazole, 5% (w/v) glycerol, 0.1% (w/v) Tween20). Proteins were eluted in buffer B (50 mM TrisHCl pH 8.0, 500 mM NaCl, 500 mM imidazole, 5% (w/v) glycerol). This and all other chromatography steps were performed on an AktaXpress system.

The eluted proteins from the HisTrapFF column were precipitated using ammonium sulphate by adding stock solution of 300 g/L ammonium sulphate to give a final concentration of 150 g/L. Samples were incubated at 4° C. for 2 h and centrifuged at 17,000 g. Resulting pellets were resuspended in buffer containing 50 mM TrisHCl pH 8.0, 500 mM NaCl, 1 mM DTT and 0.5% EDTA. His-tagged TEV protease was added to 1:1 molar ratio and samples were incubated overnight at 4° C. The reaction mix was then loaded onto a second HisTrapFF crude column equilibrated in buffer C (50 mM TrisHCl pH 8.0, 1000 mM NaCl, 20 mM imidazole, 5% (w/v) glycerol) The flowthrough containing the protein of interest with the his-tag removed was collected and the column washed with buffer B to collect uncleaved sample and TEV protease.

For mutants EcoSSB-Q152del (SEQ ID NO: 68) and EcoSSB-G117del (SEQ ID NO: 69) additional purification steps were required to remove EcoSSB-WT (SEQ ID NO: 65) contaminant carried through from E. coli expression. The flowthrough from the second HisTrapFF column was diluted tenfold with buffer D (50 mM TrisHCl pH 8.0) and loaded onto a MonoQ HR5/5 column (GE Healthcare). The flowthrough from the monoQ column containing the recombinant protein was then loaded onto a HiTrap Heparin column (GE Healthcare) equilibrated in buffer E (20 mM TrisHCl pH 7.0, 2 mM DTT). A gradient was applied over 20 CV to 100% buffer F (20 mM TrisHCl pH 7.0, 2 mM DTT, 2000 mM NaCl). The proteins eluted in approximately 360 mM NaCl (EcoSSB-Q152del, SEQ ID NO: 68) and 550 mM NaCl (EcoSSB-G117del, SEQ ID NO 69). For storage, glycerol was added to 20% volume to all samples.

### Example 2—Permanent Blocking of a Nanopore by the C-Terminus of SSB

Initial experiments designed to first assess the potential use of SSB as an additive or as a translocation facilitator protein for nanopore DNA sequencing quickly determined that addition of the E. coli SSB protein (EcoSSB-WT, SEQ ID NO: 65), in complex with ssDNA, to the cis chamber results in rapid blocking of the nanopore under positive potential. This blocking was permanent and could only be cleared on reversal of potential, unlike the transient blocking events observed for the translocation of ssDNA.

The SSB protein from E. coli SSB (EcoSSB-WT, SEQ ID NO: 65) is a very well characterised protein due to its essential role in DNA replication, repair and recombination. E. coli SSB generally exists in solution as a homotetramer in the absence of DNA. This tetrameric protein is largely a compact globular structure consisting of the N-terminal two thirds from each protein subunit, which constitutes the ssDNA binding domain. The C-terminal third of each subunit comprises a flexible glycine proline rich random peptide coil that also contains a region of highly negatively charged amino acids (Lu and Keck, 2008).

As the C-terminal third of each subunit is not required for ssDNA binding then a deletion mutant of the C-terminal third of the SSB protein was designed (EcoSSB-G117del, SEQ ID NO 69). In addition, as negatively charged polymers, such as DNA, are known to interact with nanopores then a protein that lacked only the last 15 negatively charged amino acids was also designed (EcoSSB-Q152del, SEQ ID NO: 68). To maintain the full length protein, mutations to charge neutralise the acidic residues in the C-terminus were also designed (EcoSSB-CterAla, SEQ ID NO: 66 and EcoSSB-CterNGGN, SEQ ID NO: 67).

Alignment of Escherichia coli Single Strand DNA Binding Protein (EcoSSB) Mutants
(EcoSSB-WT is SEQ ID NO: 65, EcoSSB-CterAla is SEQ ID NO: 66, EcoSSB-CterNGGN
is SEQ ID NO: 67, EcoSSB-Q152del is SEQ ID NO: 68, EcoSSB-G117del is SEQ ID NO:
69).

| | | |
|---|---|---|
| EcoSSB-WT | ASAGVNKVILVGNLGQDPEVRYMPNGGAVANITLATSESWRDKATGEMKEQTEWHRVVLF | 60 |
| EcoSSB CterAla | ASRGVNKVILVGNLGQDPEVRYMPNGGAVANITLATSESWRDKATGEMKEQTEWHRVVLF | 60 |
| EcoSSB-CLerNGGN | ASRGVNKVILVGNLGQDPEVRYMPNGGAVANITLATSESWRDKATGEMKEQTEWHRVVLF | 60 |
| EcoSSB-Q152del | ASAGVNKVILVGNLGQDPEVRYMPNGGAVANITLATSESWRDKATGEMKEQTEWHRVVLF | 60 |
| EcoSSB-G117del | ASRGVNKVILVGNLGQDPEVRYMPNGGAVANITLATSESWRDNATGEMKEQTEWHRVVLF | 60 |
| | ************************************************************ | |
| EcoSSB-WT | GKLAEVASEYLRKGSQVYIEGQLRTRKWTDQSGQDRYTTEVVVNVGGTMQMLGGRQGGGA | 120 |
| EcoSSB-CLerAla | GKLAEVASEYLRKGSQVYIEGQLRTRKWTDQSGQDRYTTEVVVNVGGTMQMLGGRQGGGA | 120 |
| EcoSSB-CterNGGN | GKLAEVASEYLRKGSQVYIEGQLRTRKWTDQSGQDRYTTEVVVNVGGTMQMLGGRQGGGA | 120 |
| EcoSSB-Q152del | GKLAEVASEYLRKGSQVYIEGQLRTRKWTDQSGQDRYTTEVVVNVGGTMQMLGGRQGGGA | 120 |
| EcoSSB-G117del | GKLAEVASEYLRKGSQVYIEGQLRTRKWTDQSGQDRYTTEVVVNVGGTMQMLGGRQG--- | 117 |
| | ************************************************************ | |
| EcoSSB-WT | PAGGNIGGGQPQGGWGQPQQPQGGNQFSGGAQSRPQQSAPAAPSNEPPMDFDDDIFF | 177 |
| EcoSSB-CterAla | PAGGNIGGGQPQGGWGQPQQPQGGNQFSGGAQSRPQQSAPAAPSNEPPMAFAAAIFF | 177 |
| EcoSSB-CterNGGN | PAGGNIGGGQPQGGWGQPQQPQGGNQFSGGAQSRPQQSAPAAPSNEPPMNFGGNIFF | 177 |
| EcoSSB-Q152del | PAGGNIGGGQPQGGWGQPQQPQGGNQFSGGAQ------------------------- | 152 |
| EcoSSB-G117del | --------------------------------------------------------- | |

To determine the improvement or otherwise of the SSB mutants on nanopore blocking, experiments were carried out to assess the blocking occurrences in the presence of ssDNA only (Table 8) and then subsequently in the presence of ssDNA+SSB (Table 9).

Electrical measurements were acquired using 128 well silicon chips (format 75 μm diameter, 20 μm depth and 250 μm pitch) which were silver plated (WO 2009/077734). Chips were initially washed with 20 mL ethanol, then 20 mL dH₂O, then 20 mL ethanol prior to CF4 plasma treatment. The chips used were then pre-treated by dip-coating, vacuum-sealed and stored at 4° C. Prior to use, the chips were allowed to warm to room temperature for at least 20 minutes.

Bilayers were formed by passing a series of slugs of 3.6 mg/mL 1,2-diphytanoyl-glycero-3-phosphocholine lipid (DPhPC, Avanti Polar Lipids, AL, USA) dissolved in 400 mM KCl, 25 mM Tris, pH 7.5, at 0.45 μL/s across the chip. Initially a lipid slug (250 μL) was flowed across the chip, followed by a 100 μL slug of air. Two further slugs of 155 μL and 150 μL of lipid solution, each separated by a 100 μL slug of air were then passed over the chip. After bilayer formation the chamber was flushed with 3 ml of buffer at a flow rate of 3 μl/s. Electrical recording of the bilayer formation was carried out at 10 kHz with an integration capacitance of 1.0 pF.

A solution of the biological nanopore was prepared using αHL-(E111N/K147N), (NN) (Stoddart, D. S., et al., (2009), Proceedings of the National Academy of Sciences of the United States of America 106, p 7702-7707) (1 μM diluted 1/1000) in 400 mM KCl, 25 mM Tris pH 7.5. A holding potential of +160 mV was applied and the solution flowed over the chip. Pores were allowed to enter bilayers until 10% occupancy (12 single pores) was achieved. The sampling rate and integration capacitance were maintained at 10 kHz and 1.0 pF respectively and the potential reduced to zero.

A programme was set which cycled through periods of positive holding potential +160 mV for 10 seconds followed by a negative holding potential of □160 mV for 50 seconds and finally a rest period where no potential was applied for 15 seconds. 70mer polyT (100 nM, SEQ ID NO: 83) and a control experiment was run for 15 minutes. The solution on the chip was then replaced with 100 nM polyT (SEQ ID NO. 83) which had been pre-incubated with 100 nM of each SSB. Blocking was then quantified by assigning the data into bins according to the proportion of time the pore is open for within the period of positive potential before blocking. It can be seen that on addition of EcoSSB-WT (SEQ ID NO: 65) the pore rapidly blocks on positive potential and remains so until the potential is reversed. In contrast to this however, somewhat surprisingly both of the C-terminal mutant proteins do not show the blocking behaviour of the wild-type enzyme. This suggests that the negative charge of the C-terminus is bringing about an interaction between the flexible C-terminal part of the SSB protein and the nanopore and so giving the permanent blockades observed.

TABLE 8
ssDNA only

| | | | | | | | Proportion | |
|---|---|---|---|---|---|---|---|---|
| | Proportion of | | | Proportion of | | | of time when | |
| | time when the | | | time when the | | | the open | |
| | open pore is | | | open pore is | | | pore is not | |
| | not blocked by | | | not blocked by | | | blocked by | |
| | DNA | % | | DNA | % | | DNA | % |
| EcoSSB- | x ≤ 0.25 | 18.60% | EcoSSB- | x ≤ 0.25 | 10.40% | EcoSSB- | x ≤ 0.25 | 19.60% |
| WT | ≤0.25 x ≤0.50 | 9.30% | CterAla | ≤0.25 x ≤0.50 | 8.30% | Q152del | ≤0.25 x ≤0.50 | 5.90% |
| | ≤0.50 x ≤0.75 | 20.90% | | ≤0.50 x ≤0.75 | 10.40% | | ≤0.50 x ≤0.75 | 9.80% |
| | x ≥ 0.75 | 51.20% | | x ≥ 0.75 | 70.80% | | x ≥ 0.75 | 64.70% |

TABLE 9
SSB:ssDNA

| | | | | | | | Proportion | |
|---|---|---|---|---|---|---|---|---|
| | Proportion of | | | Proportion of | | | of time when | |
| | time when the | | | time when the | | | the open | |
| | open pore is | | | open pore is | | | pore is not | |
| | not blocked by | | | not blocked by | | | blocked by | |
| | DNA | % | | DNA | % | | DNA | % |
| EcoSSB- | x ≤ 0.25 | 93.00% | EcoSSB- | x ≤ 0.25 | 12.50% | EcoSSB- | x ≤ 0.25 | 1.90% |
| WT | ≤0.25 x ≤0.50 | 7.00% | CterAla | ≤0.25 x ≤0.50 | 10.40% | Q152del | ≤0.25 x ≤0.50 | 11.80% |
| | ≤0.50 x ≤0.75 | 0% | | ≤0.50 x ≤0.75 | 25.00% | | ≤0.50 x ≤0.75 | 27.50% |
| | x ≥ 0.75 | 0% | | x ≥ 0.75 | 52.10% | | x ≥ 0.75 | 58.80% |

To confirm that the mutant SSB proteins are still able to interact with and bind to the DNA a small sample of EcoSSB-WT (SEQ ID NO: 65) and mutant SSB complexes (EcoSSB-Q152del, SEQ ID NO: 68) with 70mer polyT (SEQ ID NO: 83) were analysed on a 5% TBE gel, to determine presence of the bandshift typical for a protein DNA interactions (FIG. 1). It can be seen that the EcoSSB-Q152del mutant (SEQ ID NO: 68) is not impaired in its ability to form a complex with the 70mer polyT (SEQ ID NO: 83), when compared to the EcoSSB-WT (SEQ ID NO: 65). The slight shift in position of the protein DNA complex is likely due to the deletion of the C-terminus and also the charge removal.

### Example 3—Abolition of ssDNA Blocking by a Pore: DNA Complex Using SSBs that Lack a Negatively Charged C-Terminus

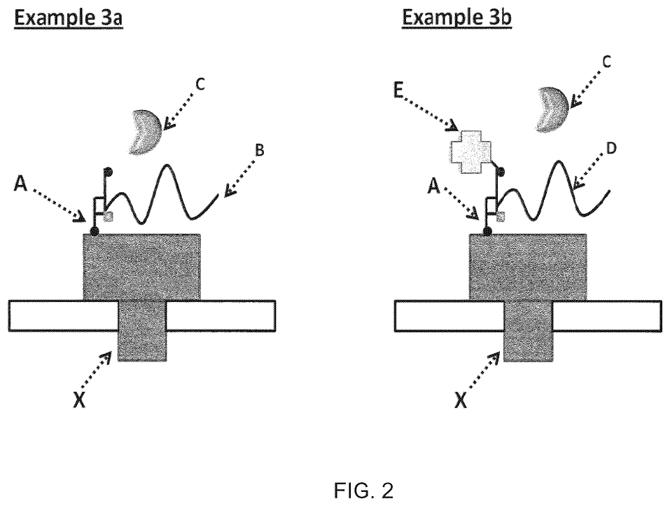

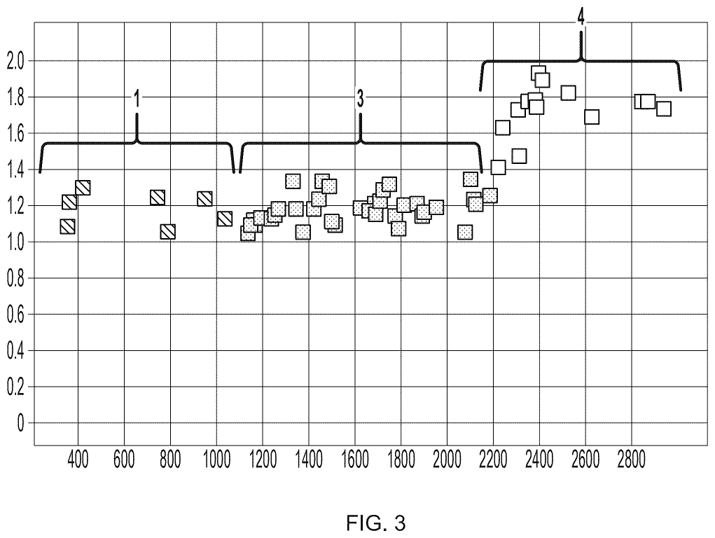

When using a nanopore as a possible sequencing platform, having control over the DNA can often be an important consideration. An example, of this can be seen in exospore sequencing where not only can the cleaved bases interact with the nanopore, as desired, but also the DNA strand itself. Interaction of the strand itself may abolish the sequencing read either through disruption of the flow of bases to the detector or by stripping the DNA analyte from the enzyme. To assay for the ability of SSB to abolish DNA nanopore interactions, an extreme case scenario was used. A DNA strand (SEQ ID NO: 78, which has a thiol group at the 5′ end of the strand) was covalently attached to a single subunit of haemolysin (SEQ ID NO: 77 with the mutations N139Q/L135C/E287C and with 5 aspartates, a Flag-tag and H6 tag to aid purification) and another strand of DNA ((comprising SEQ ID NO: 79 for Example 3a or comprising SEQ ID NO: 81 for Example 3b, both of which contain a thiol at the 5′ end and a Cy3 fluorescent tag at the 3′ end of the strand), which contains in its sequence alkyne residues (shown as n in SEQ ID NO's: 79 and 81, both of which contain a thiol at the 5′ end and a Cy3 fluorescent tag at the 3′ end of the strand) which can react with the azidohexanoic acid residues in SEQ ID NO: 78 (which also has a thiol group at the 5′ end of the strand) via click chemistry, so as to give rapid pore blocking by the DNA strand (comprising SEQ ID NO: 79 or 81 both of which contain a thiol at the 5′ end and a Cy3 fluorescent tag at the 3′ end of the strand) on applied positive potential (see FIG. 2 for the system investigate for Examples 3a and 3b). Blocking is extremely rapid due to the intramolecular concentration given by cross reacting the analyte to the protein (FIGS. 3-5).

### Sequences Used:

SEQ ID NO: 79
Thiol-GCnACGGAGACn--Cy3(where n is an alkyne)
SEQ ID NO: 81
Thiol-GCnACGGAGACn--Cy3(where n is an alkyne)

### Example 3a

Chip experiments were set-up as described in Example 2. A solution of the mutant α-haemolysin nanopore (6 subunits of SEQ ID NO: 77 with the mutation N139Q and one subunit of SEQ ID NO: 77 with the mutations N139Q/L135C/E287C, with 5 aspartates, a Flag-tag and H6 tag to aid purification and a DNA strand (SEQ ID NO: 78) reacted by its 5′ end thiol to position 287 of this subunit, which is also attached to a second piece of DNA (comprising SEQ ID NO: 79 (which has a thiol at the 5′ end and a Cy3 fluorescent tag at the 3′ end of the strand)) via click chemistry) was flowed over the chip. Multiple pores were allowed to insert into multiple bilayers until at least 10% occupancy was achieved. The sampling rate was changed to 1 kHz and the potential was cycled accordingly; 5 secs+150 mV, 1 secs 0150 mV, and 4 secs 0 mV. Time periods of 10 mins were recorded for each section; Section 1 is the control period (400 mM KCl, 25 mM Tris, 10 μM EDTA, pH 7.5), section 2 is the SSB period (10 nM, if appropriate), section 3 is the period after Mg²⁺ buffer flush (400 mM KCl, 25 mM Tris, 10 mM MgCl2, pH 7.5) and section 4 is the addition of free exonuclease I mutant enzyme (100 nM, SEQ ID NO: 80) to clear the pore by digestion of the analyte DNA (comprising SEQ ID NO: 79, which has a thiol at the 5′ end and a Cy3 fluorescent tag at the 3′ end of the strand) Data from multiple pores was collated and plotted according to block level observed. In all cases, time is given along the X-axis and the relative DNA block current level is given along the Y-axis (so 1 is current level observed when DNA is blocking the nanopore).

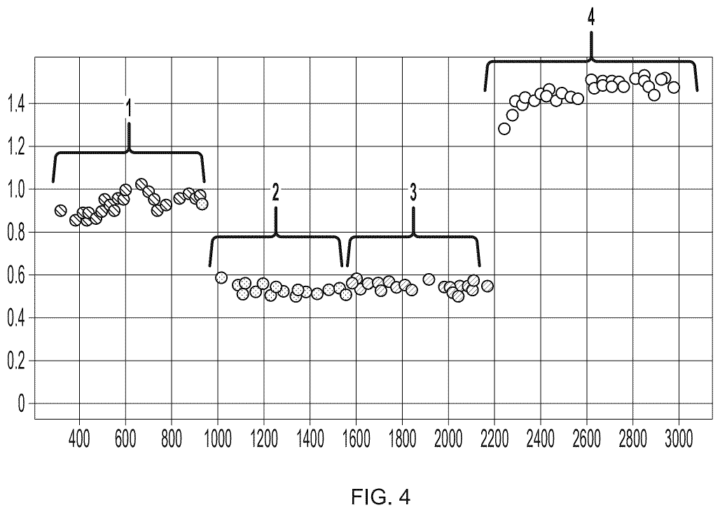

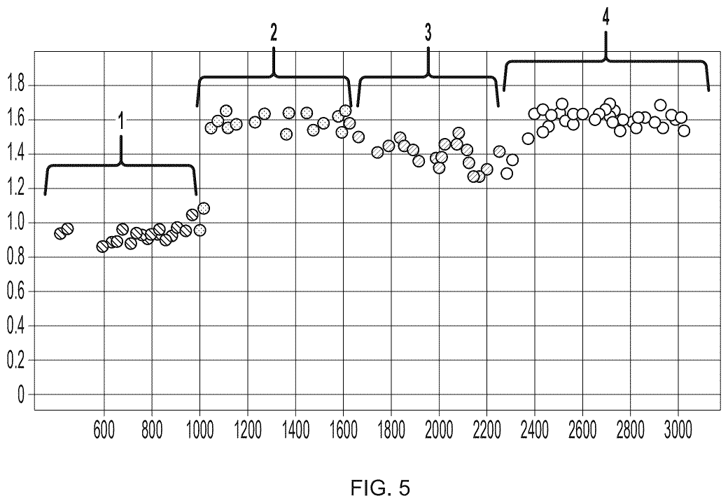

It can be seen in FIG. 3 that during the control period (section 1) the DNA (comprising SEQ ID NO: 79, which has a thiol at the 5′ end and a Cy3 fluorescent tag at the 3′ end of the strand) attached to the pore rapidly brings about a DNA block level. On addition of the free exonuclease I mutant enzyme (SEQ ID NO: 80, FIG. 3, section 4) the DNA strand (comprising SEQ ID NO: 79, which has a thiol at the 5′ end and a Cy3 fluorescent tag at the 3′ end of the strand) is digested and so the relative block level is increased, as the open pore level is now observed instead of the DNA blocking level. On addition of EcoSSB-WT (SEQ ID NO: 65, FIG. 4, section 2) the nanopore blocks to a greater current deflection to that observed for just the DNA block level (SEQ ID NO: 79 which has a thiol at the 5′ end and a Cy3 fluorescent tag at the 3′ end of the strand), so is less than 1 This is due to the interaction of the negatively charged C-terminus of the EcoSSB-WT (SEQ ID NO: 65) with the nanopore instead of the DNA (SEQ ID NO: 79 which has a thiol at the 5′ end and a Cy3 fluorescent tag at the 3′ end of the strand). Again the pore clears on digestion of the DNA strand by exonuclease I mutant enzyme (SEQ ID NO: 80, FIG. 4, section 4), as not only is the strand removed but also the EcoSSB-WT protein is no longer in close association with the nanopore, therefore, the C-terminus of EcoSSB-WT is not observed to block the pore. On addition of the Eco-SSB-Q152del (SEQ ID NO: 68, FIG. 5, section 2), however, the DNA block level is abolished, similar to that observed for addition of free exonuclease I mutant enzyme (SEQ ID NO: 80). This is because EcoSSB-Q152del (SEQ ID NO: 68) sequesters the DNA such that it cannot interact with the pore and block it, and also the protein itself does no block the pore as was observed for EcoSSB-WT (SEQ ID NO: 65).

In all cases, as the EcoSSB interaction with ssDNA is quite a stable interaction, the buffer flush does not remove the bound protein (for either EcoSSB-WT or EcoSSB-Q152del). The protein can be removed by flush with Mg²⁺ and 100 nM PolyT70mer in solution to out-compete the SSB for the DNA strand on the pore and so re-observe the DNA block levels.

### Example 3b

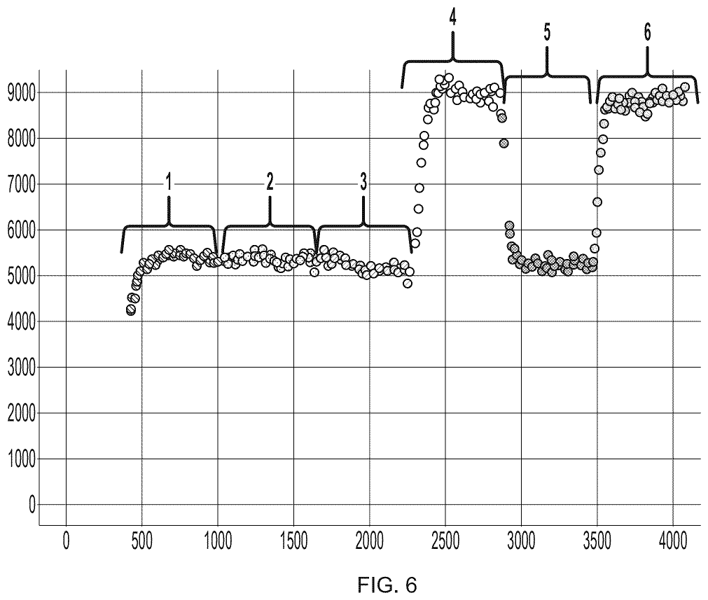

Not all single strand DNA binding proteins have a negatively charged C-terminus. However, commercially available SSBs such as EcoSSB-WT (SEQ ID NO: 65) and T4 gp32 (SEQ) ID NO: 55) all contain a negatively charged C-termini. We identified a suitable SSB from the Phi29 virus (p5) (SEQ ID NO: 64) that based on the primary structure appears to lack a C-terminal negatively charged tail, which is common to most bacterial SSBs (Gascon, Lazaro, et al. 2000). To assess the blocking of a nanopore by this protein, as well as its ability to shield this DNA from the nanopore, a similar experiment to Example 3a was carried out (FIG. 6).

Chip experiments were set-up as described in Example 2. A solution of the mutant α-haemolysin nanopore (6 subunits of SEQ ID NO: 77 with the mutation N139Q and one subunit of SEQ ID NO: 77 with the mutations N139Q/L135C/E287C and with 5 aspartates, a Flag-tag and H6 tag to aid purification and a DNA strand (SEQ ID NO: 78) reacted by its 5′ end thiol to position 287 of this subunit, which is also attached to a second DNA strand (comprising SEQ ID NO: 81 (which has a thiol at the 5′ end and a Cy3 fluorescent tag at the 3′ end of the strand), which is itself covalently attached by a thiol group at its 5′ to the mutant PhiE polymerase enzyme (SEQ ID NO: 82) at position 373) via click chemistry) was flowed over the chip. Multiple pores were allowed to insert into multiple bilayers until at least 10% occupancy was achieved. The sampling rate was changed to 1 kHz and the potential was cycled accordingly; 5 secs+150 mV, 1 secs □1150 mV, and 4 secs 0 mV. Time periods of 10 mins were recorded for each section before titration of Phi29 p5; section 1 is the control period (400 mM KCl, 25 mM Tris. 10 uM EDTA, pH 7.5), Section 2 is the 100 nM Phi29 p5 SSB (SEQ ID NO: 64) period, Section 3 is the 1 uM Phi29 p5 SSB (SEQ ID NO: 64) period, section 4 is 10 uM Phi29 p5 SSB (SEQ ID NO: 64) period, section 5 is the period after EDTA buffer flush (400 mM KCl, 25 mM Tris, 10 uM EDTA, pH 7.5) and section 6 is addition of the free exonuclease I mutant enzyme (100 nM, SEQ ID NO: 80) to clear the pore by digestion of the analyte (comprising SEQ ID NO: 81, which has a thiol at the 5′ end and a Cy3 fluorescent tag at the 3′ end of the strand).

It can be seen that during the control period (FIG. 6, section 1) the DNA attached (comprising SEQ ID NO: 81, which has a thiol at the 5′ end and a Cy3 fluorescent tag at the 3′ end of the strand) to the pore rapidly brings about a DNA block level. This blocking continues until addition of Phi29 p5 SSB (SEQ ID NO: 64) reaches 10 uM (FIG. 6, section 4), three orders of magnitude more than was required for the EcoSSB-Q152del (FIG. 5). At 10 uM concentration of Phi29 p5 SSB (SEQ ID NO. 64) the binding protein is shielding the DNA strand (comprising SEQ ID NO: 81, which has a thiol at the 5′ end and a Cy3 fluorescent tag at the 3′ end of the strand) from the pore. A flush of buffer is enough to remove the Phi29 p5 SSB (SEQ ID NO: 64, FIG. 6, section 5) as presumably this protein has very dynamic binding and so the protein is easily washed away. On addition of free exonuclease I mutant enzyme (SEQ ID NO: 80, FIG. 6, section 6) the DNA strand (comprising SEQ ID NO: 81, which has a thiol at the 5′ end and a Cy3 fluorescent tag at the 3′ end of the strand) is digested and so the relative block level is increased, as the open pore level is now observed instead of the DNA blocking level. This is similar to that seen when the Phi29 p5 SSB (SEQ ID NO: 64) bound the DNA strand (comprising SEQ ID NO: 81, which has a thiol at the 5′ end and a Cy3 fluorescent tag at the 3′ end of the strand) except that with the Phi29 p5 SSB (SEQ ID NO: 64) the strand is merely physically constrained from entering the pore and not digested.

### Example 4—Additive Effect of a Modified SSB for Strand Sequencing

Common failures of existing sequencing chemistries such as pyrosequencing can come from the fact that as templates become larger, then secondary structure within the DNA molecule affects enzyme performance. SSB's were, therefore, investigated to see if they could prevent the formation of secondary structure in strand sequencing experiments.

Electrical measurements were acquired from single MspA nanopores (ONT Ref-MspA (B2C), SEQ ID NO: 2 with mutations G75S/G77S/L88N/Q126R) inserted in 1,2-diphytanoyl-glycero-3-phosphocholine lipid (Avanti Polar Lipids) bilayers. Bilayers were formed across ˜100 μm diameter apertures in 20 μm thick PTFE films (in custom Delrin chambers) via the Montal-Mueller technique, separating two 1 mL buffered solutions. All experiments were carried out in the stated buffered solution. Single-channel currents were measured on Axopatch 200B amplifiers (Molecular Devices) equipped with 1440A digitizers. Platinum electrodes are connected to the buffered solutions so that the cis compartment (to which both nanopore and enzyme/DNA are added) is connected to the ground of the Axopatch headstage, and the trans compartment is connected to the active electrode of the headstage.

After achieving a single pore in the bilayer (buffer solution=400 mM NaCl, 100 mM HEPES pH 8.0, 10 mM potassium ferrocyanide, 10 mM potassium ferricyanide, MspA nanopore—E. coli MS (B1-G75S-G77S-L88N-Q126R) 8 MspA (SEQ ID NO: 2 with the mutations G75S/G77S/L88N/Q126R), ATP (1 mM) and MgCl₂ (1 mM) were added to the cis compartment of the electrophysiology chamber. A control experiment was run at +140 mV. The 5 KB phiX DNA (SEQ ID NO's: 70 (which has 50 spacer units at the 5′ end of the sequence), 56 and 57 (which at the 3′ end of the sequence has six iSp18 spacers attached to two thymine residues and a 3′ cholesterol TEG), 0.5 nM) was then added to the cis compartment of the electrophysiology chamber and a further experiment run to check for DNA translocation events. The helicase Hel308Tga (SEQ ID NO: 16, 1 μM) was then added to the cis compartment and a further control experiment was run. Finally, SSB (either EcoSSB-WT (SEQ ID NO: 65) or EcoSSB-Q152del (SEQ ID NO: 68) at 1 μM). Experiments were carried out at a constant potential of +140 mV.

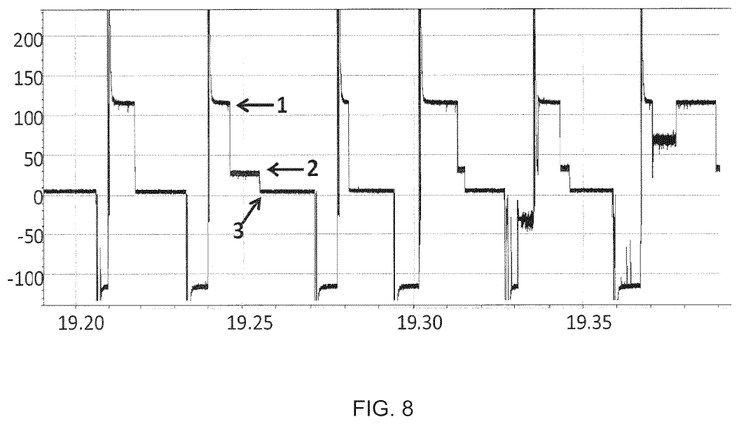

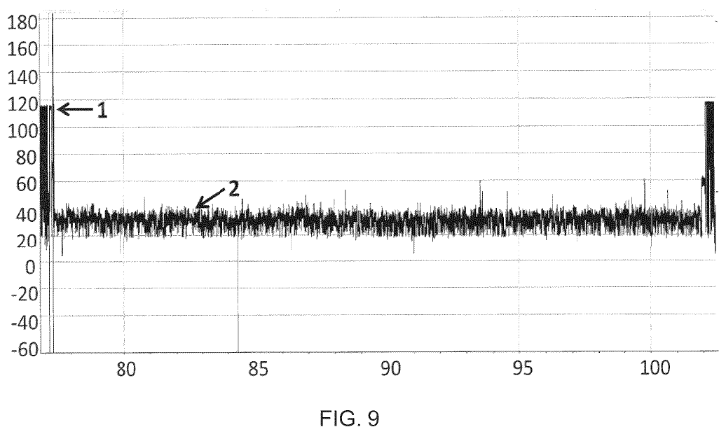

Previous attempts using a Hel308 enzyme homologue, from T. gammatolerans, to process a 5 kb dsDNA template (SEQ ID NO's: 70 (which has 50 spacer units at the 5′ end of the sequence), 56 and 57 (which at the 3′ end of the sequence has six iSp18 spacers attached to two thymine residues and a 3′ cholesterol TEG)), with an abasic leader for capture by the nanopore, proved difficult to obtain. Addition of EcoSSB-WT (SEQ ID NO: 65) again appeared to cause the pore to block to a steady level (See FIG. 8, level 3). However, on addition of EcoSS-Q152del (SEQ ID NO: 68) helicase controlled DNA movement was observed that seemed to process the strand all the way to the end (FIG. 9 shows one 5 kB DNA helicase controlled DNA movement).

The fact that the EcoSSB-Q152del (SEQ ID NO: 68) seemingly allows the enzyme to process 5 kb of continuous data again indicates that an SSB protein lacking a C-terminal negative charge could be a suitable additive for nanopore DNA sequencing.

### Example 5

This Example compares the DNA binding ability of various transport control proteins, such as a helicase, a helicase dimer, a helicase attached to a nucleic acid binding domain or a helicase attached to an enzyme, and constructs, comprising a transport control protein attached to an SSB, using a fluorescence based assay.

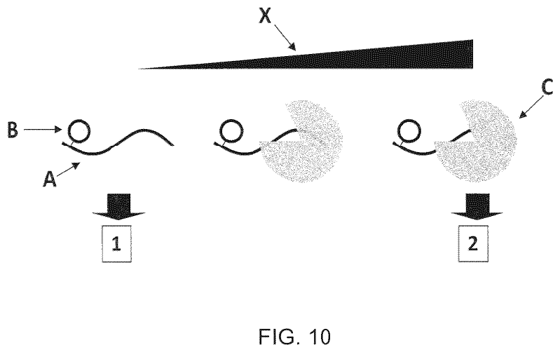

A custom fluorescent substrate was used to assay the ability of various transport control proteins and constructs to bind to single-stranded DNA. The 88 nt single-stranded DNA substrate (1 nM final, SEQ ID NO: 73) has a carboxyfluorescein (FAM) base at its 5′ end. As the transport control protein or construct binds to the oligonuclotides in a buffered solution (400 mM NaCl, 10 mM Hepes, pH8 0, 1 mM MgCl₂), the fluorescence anisotropy (a property relating to the rate of free rotation of the oligonucleotide in solution) increases. The lower the amount of transport control protein or construct needed to affect an increase in anisotropy, the tighter the binding affinity between the DNA and transport control protein or construct (FIG. 10).

The transport control proteins that were tested include:
1) Hel308 Mbu monomer (SEQ ID NO: 10);
2) Hel308 Mbu A700C 2 kDa dimer (where each monomer unit comprises SEQ ID NO: with the mutation A700C, with one monomer unit being linked to the other via position 700 of each monomer unit using a 2 kDa PEG linker);
3) Hel308 Mbu-GTGSGA-(HhH)2 (where a helicase monomer unit (SEQ ID NO: 10) is attached by the linker sequence GTGSGA to a (HhH)2 domain (SEQ ID NO: 74));
4) Hel308 Mbu-GTGSGA-(HhH)2-(HhH)2 (where a helicase monomer unit (SEQ ID NO: 10) is attached by the linker sequence GTGSGA to a (HhH)2-(HhH)2 domain (SEQ ID NO: 75)); and
5) Hel308 Mbu-GTGSGA-UL42HV1-1320Del (where a helicase monomer unit (SEQ ID NO: 10) is attached by the linker sequence GTGSGA to UL42HV1-1320Del (SEQ ID NO: 76)).

The constructs that were tested in the assay include:
a) Hel308 Mbu-GTGSGA-gp32RB69CD (where a helicase monomer unit (SEQ ID NO: 10) is attached by the linker sequence GTGSGA to the SSB gp32RB69CD (SEQ ID NO: 59));
b) Hel308 Mbu-GTGSGA-gp2.5T7-R211Del (where a helicase monomer unit (SEQ ID NO: 10) is attached by the linker sequence GTGSGA to the SSB gp2.5T7-R211Del (SEQ ID NO: 60)); and
c) gp32-RB69CD-GTGSGT-Hel308 Mbu (where the SSB gp32-RB69CD (SEQ ID NO: 59) is attached by the linker sequence GTGSGT to the helicase monomer unit (SEQ ID NO: 10)).

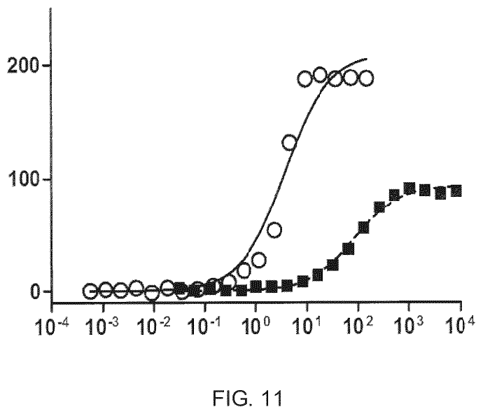

FIG. 11 shows the change in anisotropy of the DNA oligonucleotide (SEQ ID NO: 73, which has a carboxyfluorescein base at its 5′ end) with increasing amounts of Hel308 Mbu A700C 2 kDa dimer (empty circles) in comparison with the Hel308 Mbu monomer (black squares).

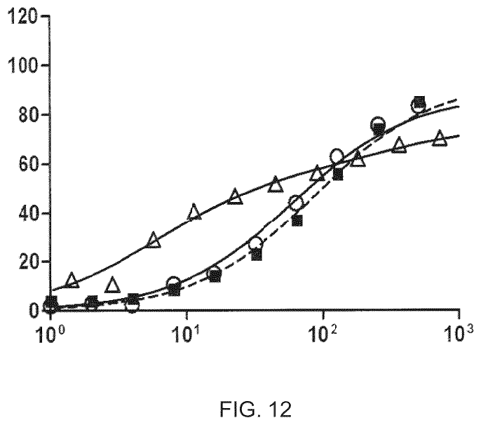

FIG. 12 shows the change in anisotropy of the DNA oligonucleotide (SEQ ID NO. 73, which has a carboxyfluorescein base at its 5′ end) with increasing amounts of Hel308 Mbu-GTGSGA-(HhH)2 (empty circles) and Hel308 Mbu-GTGSGA-(HhH)2-(HhH)2 (empty triangles) in comparison with the Hel308 Mbu monomer (black squares).

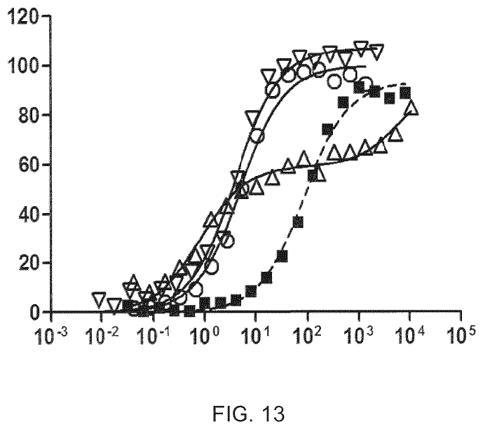

FIG. 13 shows the change in anisotropy of the DNA oligonucleotide (SEQ ID NO: 73, which has a carboxyfluorescein base at its 5′ end) with increasing amounts of Hel308 Mbu-GTGSGA-UL42HV1-1320Del (empty circles), Hel308 Mbu-GTGSGA-gp32RB69CD (empty triangles pointing up) and Hel308 Mbu-GTGSGA-gp2.5T7-R211Del (empty triangles pointing down) in comparison with the Hel308 Mbu monomer (black squares).

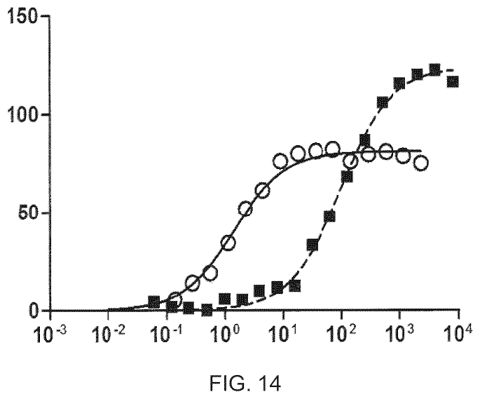

FIG. 14 shows the change in anisotropy of the DNA oligonucleotide (SEQ ID NO: 73, which has a carboxyfluorescein base at its 5′ end) with increasing amounts of (gp32-RB69CD)-Hel308 Mbu (empty circles) in comparison to the Hel308 Mbu monomer (black squares).

All of the transport control proteins and constructs that were investigated showed an increase in anisotropy at a lower concentration than the transport control protein, Hel308 Mbu monomer (SEQ ID NO: 10).

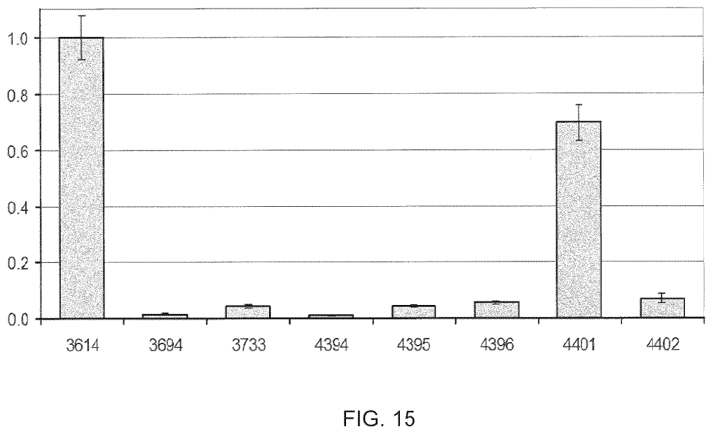

FIG. 15 shows the relative equilibrium dissociation constants (K_{d}) (with respect to Hel308 Mbu monomer SEQ ID NO: 10 whose data corresponds to column number 3614 in FIG. 15) for various transport control proteins and constructs obtained through fitting two phase dissociation binding curves through the data shown in FIGS. 11-14, using Graphpad Prism software. All of the other transport control proteins and constructs that were tested show a lower equilibrium dissociation constant than the Hel308 Mbu monomer alone. Therefore, the other transport control proteins and constructs tested all showed stronger binding to DNA than the Hel308 Mbu monomer.

1.-38. (canceled)

39. A method of unblocking a transmembrane protein pore, the method comprising:
(i) contacting the transmembrane protein pore with a polypeptide under an applied electrical potential;
(ii) measuring a current through the transmembrane protein pore indicative of blocking of the transmembrane protein pore by the polypeptide; and
(iii) reversing the applied electrical potential to clear the polypeptide from the transmembrane protein pore, thereby unblocking the transmembrane protein pore.

40. The method of claim 39, wherein the polypeptide comprises a charged tail that contacts the transmembrane protein pore.

41. The method of claim 39, wherein the polypeptide is a protein.

42. The method of claim 41, wherein the protein is a single-stranded binding protein (SSB).

43. The method of claim 39, wherein measuring a current through the transmembrane protein pore comprises measuring an ion current.

44. The method of claim 39, wherein the applied electrical potential is a voltage potential.

45. The method of claim 44, wherein the voltage potential is in the range of −400 mV to +400 mV.

46. The method of claim 39, wherein reversing the applied electrical potential comprises applying a cycle of a period of a positive holding potential followed by a period of a negative holding potential.

47. The method of claim 46, wherein the negative holding potential is followed by a rest period where no potential is applied.

48. The method of claim 39, wherein the transmembrane protein pore is provided in an array of individually addressable transmembrane protein pores.

49. The method of claim 39, wherein the transmembrane protein pore is selected from hemolysin, leukocidin, Mycobacterium smegmatis porin A (MspA), MspB, MspC, MspD, outer membrane phospholipase A, Neisseria autotransporter lipoprotein (NaIP) and WZA.

50. The method of claim 39, wherein the barrel or channel of the transmembrane protein pore has a diameter of less than 7 nm at its narrowest point.
